(19) United States
(12) Patent Application Publication
(10) Pub. No.: US 2009/0325960 A1
(43) Pub. Date: Dec. 31, 2009

(54) METHOD FOR TREATING INFLAMMATORY DISEASES USING RHO KINASE INHIBITOR COMPOUNDS

(76) Inventors: Emilee H. Fulcher, Cary, NC (US); John W. Lampe, Cary, NC (US); Tomas Navratil, Carrboro, NC (US); Ward M. Peterson, Morrisville, NC (US)

Correspondence Address: HOWERY LLP, C/O IP DOCKETING DEPARTMENT, 2941 FAIRVIEW PARK DRIVE SUITE 200, FALLS CHURCH, VA, 22042, US

(21) Appl. No.: 12/492,869

(22) Filed: Jun. 26, 2009

Related U.S. Application Data
(60) Provisional application No. 61/075,873, filed on Jun. 26, 2008.
(60) Provisional application No. 61/169,239, filed on Apr. 14, 2009.
(60) Provisional application No. 61/169,639, filed on Apr. 15, 2009.
(60) Provisional application No. 61/169,635, filed on Apr. 15, 2009.

(51) Int. Cl. A61K 31/5377; A61K 31/454; A61K 31/416; A61K 31/496; A61K 31/4725; A61P 19/02; A61P 1/00

(52) U.S. Cl. 514/236.5; 514/322; 514/406; 514/253.09; 514/310

(57) ABSTRACT

This invention is directed to methods of preventing or treating diseases or conditions associated with excessive cell proliferation, remodeling, edema and inflammation. Particularly, this invention is directed to methods of treating inflammatory diseases or conditions such as rheumatoid arthritis and inflammatory bowel disease. The method comprises identifying a subject in need of the treatment, and administering to the subject an effective amount of a compound of a novel rho kinase inhibitor compound to treat the disease.

This application claims the benefit of U.S. Provisional Application Nos. 61/075,873, filed Jun. 26, 2008; 61/169,239, filed Apr. 14, 2009; 61/169,639, filed Apr. 15, 2009; and 61/169,635, filed Apr. 15, 2009; which are incorporated herein by reference in their entirety.

### TECHNICAL FIELD

This invention relates to methods of preventing or treating diseases or conditions associated with excessive cell proliferation, remodeling, edema and inflammation. Particularly, this invention relates to methods of treating inflammatory diseases or conditions such as rheumatoid arthritis and inflammatory bowel disease, using novel rho kinase inhibitor compounds.

### BACKGROUND OF THE INVENTION

### Rho Kinase as a Target

The Rho family of small GTP binding proteins can be activated by several extracellular stimuli such as growth factors, hormones and mechanic stress and function as a molecular signaling switch by cycling between an inactive GDP-bound form and an active GTP-bound form to elicit cellular responses. Rho kinase (ROCK) functions as a key downstream mediator of Rho and exists as two isoforms (ROCK 1 and ROCK 2) that are ubiquitously expressed. ROCKs are serine/threonine kinases that regulate the function of a number of substrates including cytoskeletal proteins such as adducin, moesin, Na⁺—H⁺ exchanger 1 (NHE1), LIM-kinase and vimentin, contractile proteins such as the myosin light chain phosphatase binding subunit (MYPT-1), CPI-17, myosin light chain and calponin, microtubule associated proteins such as Tau and MAP-2, neuronal growth cone associate proteins such as CRMP-2, signaling proteins such as PTEN and transcription factors such as serum response factor (Loirand et al, Circ Res 98:322-334, 2006). ROCK is also required for cellular transformation induced by RhoA. As a key intermediary of multiple signaling pathways, ROCK regulates a diverse array of cellular phenomena including cytoskeletal rearrangement, actin stress fiber formation, proliferation, chemotaxis, cytokinesis, cytokine and chemokine secretion, endothelial or epithelial cell junction integrity, apoptosis, transcriptional activation and smooth muscle contraction. As a result of these cellular actions, ROCK regulates physiologic processes such as vasoconstriction, bronchoconstriction, tissue remodeling, inflammation, edema, platelet aggregation and proliferative disorders.

One well documented example of ROCK activity is in smooth muscle contraction. In smooth muscle cells ROCK mediates calcium sensitization and smooth muscle contraction. Agonists (noradrenaline, acetylcholine, endothelin, etc.) that bind to G protein coupled receptors produce contraction by increasing both the cytosolic Ca²⁺ concentration and the Ca²⁺ sensitivity of the contractile apparatus. The Ca²⁺-sensitizing effect of smooth muscle constricting agents is ascribed to ROCK-mediated phosphorylation of MYPT-1, the regulatory subunit of myosin light chain phosphatase (MLCP), which inhibits the activity of MLCP resulting in enhanced phosphorylation of the myosin light chain and smooth muscle contraction (WO 2005/003101A2, WO 2005/034866A2).

### Rheumatoid Arthritis

Rheumatoid arthritis (RA) is classified as an inflammatory disorder resulting from acute and chronic inflammation in the synovium that is associated with a proliferative and destructive process in the joint tissue (Harris, E D. Overview of the management of rheumatoid arthritis. In:UpToDate, Schur, P H (Ed), UpToDate, Wellesley, Mass., 2008). One of the earliest pathogenic responses in RA is the generation of new blood vessels, angiogenesis, which is recognized as being fundamental to establishing and perpetuation of the disease (Harris, E D. Pathogenesis of rheumatoid arthritis. In:UpToDate, Schur, P H (Ed), UpToDate, Wellesley, Mass., 2008). As the new vessels develop, inflammatory cells accumulate in the synovium and synovial fluid and release pro-inflammatory cytokines that propagate the inflammatory response and lead to tissue destruction. Among the inflammatory cells present in the synovium of RA patients are eosinophils, neutrophils, T-lymphocytes, and importantly monocytes and macrophages which secrete TNF-α and IL-1β, two cytokines that play a central role in the pathophysiology of RA (Goldblatt et al. Clinical and Experimental Immunology, 140:195-204, 2005). The current line of therapy includes the use of disease modifying antirheumatic drugs (DMARDs), glucocorticoids, anticytokine therapies, methotrexate, and others. However, despite the availability of these therapies approximately 30 percent of patients with RA fail to respond adequately to therapy (Helfgott, S M. Evaluation and medical management of end-stage rheumatoid arthritis. In:UpToDate, Schur, P H (Ed), UpToDate, Wellesley, Mass., 2008).

Y-27632 and Fasudil, known ROCK inhibitors, have been demonstrated to suppress expression of IL-1β, TNFα, and various other cytokines from several types of cells in the vasculature, including monocytes, endothelial cells, and lymphocytes (Doe C et al. The Journal of Pharmacology and Experimental Therapeutics, 320:89-98, 2007 and Segain J et al, Gastroenterology, 124:1180-1187, 2003). In an in vivo VEGF-induced angiogenesis model, it has been shown that in the presence of 100 μM Fasudil, new vessel formation was prevented and the fluorescence was reduced to the basic level, (Yin L et al. Mol Cancer Ther, 6: 1517-1525, 2007).

### Inflammatory Bowel Disease

Inflammatory Bowel Diseases (IBD) consists of Crohn's disease and ulcerative colitis which produce inflammation in the digestive tract. Crohn's disease is characterized by a transmural, granulomatous inflammation occurring anywhere in the alimentary canal, but is usually centered in the terminal ileum and ascending colon. The inflammation associated with Crohn's disease is characterized by “skip lesions” consisting of areas of inflammation alternating with areas of normal mucosa. The affected area of bowel in Crohn's is marked by erythema, edema, and increased friability. When inflammation is present for a long time (chronic), it sometimes can cause scarring (fibrosis). Clinical signs/symptoms of Crohn's disease can include but are not limited to: cachexia, and poor growth, abdominal pain, draining fisulae, rectal prolapse and dehydration. Ulcerative colitis, in contrast, is marked by a superficial inflammation causing epithelial cell destruction (ulceration) that is centered in the rectum and colon. Unlike Crohn's disease, ulcerative colitis only affects one section of the inner lining of the colon starting from the rectum. Ulcerative colitis can be classified into several areas of the digestive tract, but contain common symptoms of bloody or loose stools, inflammation, abdominal pains, dehydration, and weight loss.

The idiopathic inflammatory bowel diseases (Crohn's disease and ulcerative colitis) are due to inappropriate and/or excessive responses to antigens present in the normal bacterial micro flora. Bacterial products, such as lipopolysaccharide (LPS), stimulate the recruitment of inflammatory cells and the release of cytokines (Segain, J P Gastroenterology, 124: 1180-1187, 2003). This recruitment of inflammatory cells and release of cytokines contribute to the inflammation of the digestive tract. Defect in epithelial barrier function is a common occurrence in those with inflammatory bowel disease, and may contribute PMN infiltration into the intestinal mucosa. Disease activity in IBD is linked to the transepithelial influx of neutrophils, ultimately leading to the formation of crypt abscesses in the intestinal lumen (Kucharzik, T Am. J. of Path., 159: 2001-2009, 2001). Symptoms associated with inflammatory bowel disease affect the daily lives and quality of people. The prevalence of IBS in North America estimated from population-based studies is approximately 10 to 15 percent and in Europe is found to be 11.5 percent (Chun, et. al. Clinical manifestations and diagnosis of irritable bowel syndrome. In: UpToDate, Lamont J T (Ed), UpToDate, Wellesley, Mass., 2008). In many studies, it has been suggested that relatives with a first-degree relation to a person affected with IBD will have a 4 to 20 times higher chance of acquiring the disease over the general population (Podolsky, P D, N Engl J Med: 347(6): 417-29, 2002). Out of those people with symptoms of IBD, only 15 percent seek medical attention. Regardless of the small amount of people seeking medical attention, IBD results in 25 to 50 percent of all gastroenterologist referrals. IBD affects the financial situation for many people inflicted with the condition. IBD is the second highest cause of work absentees after the common cold and has been interrelated to higher health care costs with an annual total of $30 billion (Chun, et. al. Clinical manifestations and diagnosis of irritable bowel syndrome. In: UpToDate, Lamont J T (Ed), UpToDate, Wellesley, Mass., 2008).

Present treatments for inflammatory bowel disease include anti-inflammatory drugs, immune system suppressors, or surgery. Anti-inflammatory drugs consist of azulfidines, colazals, salicylate, and corticosteroids. While these drugs prove somewhat beneficial, there are numerous side effects such as vomiting, increased diarrhea, high blood pressure and diabetes, bone fractures, mild kidney inflammation, and stunted growth and can also be used only short-term. After long term use of corticosteroids, side effects can include thinning of the bone and skin, infections, diabetes, muscle wasting, rounding of faces, psychiatric disturbances, and destruction of hip joints. Immune system repressors can be used for longer amounts of time, but because the drugs suppress the immune system, fatal infection and contraction of other immune diseases is more prevalent. Surgery is recommended for those who do not respond to oral medications. Surgery often makes daily tasks difficult due to protocolectomy and the requirement of wearing a small bag to collect stools.

The use of a prototype non-potent Rho-kinase inhibitor, Y27632 in an animal model of TNBS-induced colitis, a model of IBD, has been disclosed (Segain, J P Gastroenterology, 124: 1180-1187, 2003).

There is a need for an effective or improved method for treating inflammatory diseases such as rheumatoid arthritis and inflammatory bowel disease.

### SUMMARY OF THE INVENTION

The present invention is directed to methods of preventing or treating diseases or conditions associated with excessive cell proliferation, remodeling, edema and inflammation. Particularly, this invention is directed to methods of treating inflammatory diseases or disorders associated with inflammatory conditions such as rheumatoid arthritis and inflammatory bowel disease. The method comprises identifying a subject in need of the treatment, and administering to the subject an effective amount of a novel rho kinase inhibitor compound of Formula I or Formula II to treat the disease.

The active compound is delivered to a subject by systemic administration or local administration.

### BRIEF DESCRIPTION OF THE DRAWINGS

FIG. 1 shows the % inhibition of IL-1β Secretion in Human Monocytes by Rho Kinase Inhibitors. Data represent the mean±SD of at least n=2 experiments.

FIG. 2 shows the murine eosinophil chemotaxis. The data reported are mean number of migrated eosinophils per high power view field±SEM. Average of at least 2 view fields per well, each treatment ran in triplicate.

FIG. 3 shows the human eosinophil chemotaxis. The data reported are mean number of migrated eosinophils per high power view field±SEM. Average of at least 3 view fields per well, each treatment ran in duplicate.

FIG. 4 shows percent of FBS induced proliferation. Each compound was tested at 30 uM and challenged with 10% FBS with an n=3. * indicates n=5.

FIG. 5 shows the anti-inflammatory dosing paradigm.

FIG. 6 shows the eosinophils per mL in ova-sensitized, ova-challenged, mice treated with Compound 2.038, mice treated with Compound 1.131 and normal mice.

FIG. 7 shows the dose response effect of Compound 1.091 on eosinophil influx when dosed to ova-sensitized, ova-challenged mice, *, p<0.05 when compared to ova-sensitized, ova-challenged mice using Student's t-test.

FIG. 8 shows the concentration of IL-5 (pg/mL) in BALF of (1) ova-sensitized, ova-challenged mice, (2) ova-sensitized, ova-challenged mice treated with Compound 2.038 (15 μmol/kg/oral), and (3) normal, saline-sensitized mice. Dashed line indicates the lower limit of detection for the cytokine of interest. Data represent mean±SEM, n=10 for ova-sensitized, ova-challenged mice, treated or untreated; n=5 for normal mice.

FIG. 9 shows the concentration of Eotaxin (pg/mL) in BALF of (1) ova-sensitized, ova-challenged, (2) ova-sensitized, ova-challenged mice treated with Compound 2.038 (15 μmol/kg/oral), and (3) normal, saline-sensitized mice. Dashed line indicates the lower limit of detection for the cytokine of interest. Data represent mean±SEM, n=10 for ova-sensitized, ova-challenged mice, treated or untreated; n=5 for normal mice.

FIG. 10 shows the concentration of IL-13 (pg/mL) in BALF of (1) ova-sensitized, ova-challenged, (2) ova-sensitized, ova-challenged mice treated with Compound 2.038 (15 μmol/kg/oral), and (3) normal, saline-sensitized mice. Dashed line indicates the lower limit of detection for the cytokine of interest. Data represent mean±SEM, n=10 for ova-sensitized, ova-challenged mice, treated or untreated; n=5 for normal mice.

FIG. 11 shows the dose response effect of Compound 1.091 on airway hyperreactivity when dosed using the anti-inflammatory dosing paradigm on Days 27 to 30. *, p<0.05 using statistical analysis described in Example 11.

FIG. 12 shows the dose-dependent inhibition of LPS-induced neutrophilia by Compound 1.091 when dosed intratracheally to mice. Data are reported as cells/ml and are mean±SEM. *, p<0.05 when compared to mice treated with LPS alone using Student's t-test.

FIG. 13 shows the reduction of IL-1β levels in BALF from LPS-challenged animals by Compound 1.091 or Compound 2.059. Data are reported as pg/mL of IL-11 and are mean±SEM

FIGS. 14A and 14B show [³H]-thymidine incorporation in primary human LAM-derived cells. Cells were treated with vehicle alone (control) or with 10 μM of Compound 1.132, Compound 2.066 or Compound 1.161. Experiments were performed on two separate cell lines, LAM1 cells (FIG. 14A) and LAM2 cells (FIG. 14B). Data are reported as counts per minute (CPM) of incorporated [³H]-thymidine and are mean±SEM.

### DETAILED DESCRIPTION OF THE INVENTION

### Definitions

When present, unless otherwise specified, the following terms are generally defined as, but are not limited to, the following:

Halo substituents are taken from fluorine, chlorine, bromine, and iodine.

“Alkyl” refers to groups of from 1 to 12 carbon atoms inclusively, either straight chained or branched, more preferably from 1 to 8 carbon atoms inclusively, and most preferably 1 to 6 carbon atoms inclusively.

“Alkenyl” refers to groups of from 2 to 12 carbon atoms inclusively, either straight or branched containing at least one double bond but optionally containing more than one double bond.

“Alkynyl” refers to groups of from 2 to 12 carbon atoms inclusively, either straight or branched containing at least one triple bond but optionally containing more than one triple bond, and additionally optionally containing one or more double bonded moieties.

“Alkoxy” refers to the group alkyl-O— wherein the alkyl group is as defined above including optionally substituted alkyl groups as also defined above.

“Alkenoxy” refers to the group alkenyl-O— wherein the alkenyl group is as defined above including optionally substituted alkenyl groups as also defined above.

“Alkynoxy” refers to the group alkynyl-O— wherein the alkynyl group is as defined above including optionally substituted alkynyl groups as also defined above.

“Aryl” refers to an unsaturated aromatic carbocyclic group of from 6 to 14 carbon atoms inclusively having a single ring (e.g., phenyl) or multiple condensed rings (e.g., naphthyl or anthryl). Preferred aryls include phenyl, naphthyl and the like.

“Arylalkyl” refers to aryl-alkyl-groups preferably having from 1 to 6 carbon atoms inclusively in the alkyl moiety and from 6 to 10 carbon atoms inclusively in the aryl moiety. Such arylalkyl groups are exemplified by benzyl, phenethyl and the like.

“Arylalkenyl” refers to aryl-alkenyl-groups preferably having from 2 to 6 carbon atoms in the alkenyl moiety and from 6 to 10 carbon atoms inclusively in the aryl moiety.

“Arylalkynyl” refers to aryl-alkynyl-groups preferably having from 2 to 6 carbon atoms inclusively in the alkynyl moiety and from 6 to 10 carbon atoms inclusively in the aryl moiety.

“Cycloalkyl” refers to cyclic alkyl groups of from 3 to 12 carbon atoms inclusively having a single cyclic ring or multiple condensed rings which can be optionally substituted with from 1 to 3 alkyl groups. Such cycloalkyl groups include, by way of example, single ring structures such as cyclopropyl, cyclobutyl, cyclopentyl, cyclooctyl, 1-methylcyclopropyl, 2-methylcyclopentyl, 2-methylcyclooctyl, and the like, or multiple ring structures such as adamantyl, and the like.

“Cycloalkenyl” refers to cyclic alkenyl groups of from 4 to 12 carbon atoms inclusively having a single cyclic ring or multiple condensed rings and at least one point of internal unsaturation, which can be optionally substituted with from 1 to 3 alkyl groups. Examples of suitable cycloalkenyl groups include, for instance, cyclobut-2-enyl, cyclopent-3-enyl, cyclooct-3-enyl and the like.

“Cycloalkylalkyl” refers to cycloalkyl-alkyl-groups preferably having from 1 to 6 carbon atoms inclusively in the alkyl moiety and from 6 to 10 carbon atoms inclusively in the cycloalkyl moiety. Such cycloalkylalkyl groups are exemplified by cyclopropylmethyl, cyclohexylethyl and the like.

“Cycloalkylalkenyl” refers to cycloalkyl-alkenyl-groups preferably having from 2 to 6 carbon atoms inclusively in the alkenyl moiety and from 6 to 10 carbon atoms inclusively in the cycloalkyl moiety. Such cycloalkylalkenyl groups are exemplified by cyclohexylethenyl and the like.

“Cycloalkylalkynyl” refers to cycloalkyl-alkynyl-groups preferably having from 2 to 6 carbon atoms inclusively in the alkynyl moiety and from 6 to 10 carbon atoms inclusively in the cycloalkyl moiety. Such cycloalkylalkynyl groups are exemplified by cyclopropylethynyl and the like.

“Heteroaryl” refers to a monovalent aromatic heterocyclic group of from 1 to 10 carbon atoms inclusively and 1 to 4 heteroatoms inclusively selected from oxygen, nitrogen and sulfur within the ring. Such heteroaryl groups can have a single ring (e.g., pyridyl or furyl) or multiple condensed rings (e.g., indolizinyl or benzothienyl).

“Heteroarylalkyl” refers to heteroaryl-alkyl-groups preferably having from 1 to 6 carbon atoms inclusively in the alkyl moiety and from 6 to 10 atoms inclusively in the heteroaryl moiety. Such heteroarylalkyl groups are exemplified by pyridylmethyl and the like.

“Heteroarylalkenyl” refers to heteroaryl-alkenyl-groups preferably having from 2 to 6 carbon atoms inclusively in the alkenyl moiety and from 6 to 10 atoms inclusively in the heteroaryl moiety.

“Heteroarylalkynyl” refers to heteroaryl-alkynyl-groups preferably having from 2 to 6 carbon atoms inclusively in the alkynyl moiety and from 6 to 10 atoms inclusively in the heteroaryl moiety.

“Heterocycle” refers to a saturated or unsaturated group having a single ring or multiple condensed rings, from 1 to 8 carbon atoms inclusively and from 1 to 4 hetero atoms inclusively selected from nitrogen, sulfur or oxygen within the ring. Such heterocyclic groups can have a single ring (e.g., piperidinyl or tetrahydrofuryl) or multiple condensed rings (e.g., indolinyl, dihydrobenzofuran or quinuclidinyl). Preferred heterocycles include piperidinyl, pyrrolidinyl and tetrahydrofuryl.

“Heterocycle-alkyl” refers to heterocycle-alkyl-groups preferably having from 1 to 6 carbon atoms inclusively in the alkyl moiety and from 6 to 10 atoms inclusively in the heterocycle moiety. Such heterocycle-alkyl groups are exemplified by morpholino-ethyl, pyrrolidinylmethyl, and the like.

“Heterocycle-alkenyl” refers to heterocycle-alkenyl-groups preferably having from 2 to 6 carbon atoms inclusively in the alkenyl moiety and from 6 to 10 atoms inclusively in the heterocycle moiety.

“Heterocycle-alkynyl” refers to heterocycle-alkynyl-groups preferably having from 2 to 6 carbon atoms inclusively in the alkynyl moiety and from 6 to 10 atoms inclusively in the heterocycle moiety.

Examples of heterocycles and heteroaryls include, but are not limited to, furan, thiophene, thiazole, oxazole, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, pyrrolidine, indoline and the like.

Unless otherwise specified, positions occupied by hydrogen in the foregoing groups can be further substituted with substituents exemplified by, but not limited to, hydroxy, oxo, nitro, methoxy, ethoxy, alkoxy, substituted alkoxy, trifluoromethoxy, haloalkoxy, fluoro, chloro, bromo, iodo, halo, methyl, ethyl, propyl, butyl, alkyl, alkenyl, alkynyl, substituted alkyl, trifluoromethyl, haloalkyl, hydroxyalkyl, alkoxyalkyl, thio, alkylthio, acyl, carboxy, alkoxycarbonyl, carboxamido, substituted carboxamido, alkylsulfonyl, alkylsulfinyl, alkylsulfonylamino, sulfonamido, substituted sulfonamido, cyano, amino, substituted amino, alkylamino, dialkylamino, aminoalkyl, acylamino, amidino, amidoximo, hydroxamoyl, phenyl, aryl, substituted aryl, aryloxy, arylalkyl, arylalkenyl, arylalkynyl, pyridyl, imidazolyl, heteroaryl, substituted heteroaryl, heteroaryloxy, heteroarylalkyl, heteroarylalkenyl, heteroarylalkynyl, cyclopropyl, cyclobutyl, cyclopentyl, cyclohexyl, cycloalkyl, cycloalkenyl, cycloalkylalkyl, substituted cycloalkyl, cycloalkyloxy, pyrrolidinyl, piperidinyl, morpholino, heterocycle, (heterocycle)oxy, and (heterocycle)alkyl; and preferred heteroatoms are oxygen, nitrogen, and sulfur. It is understood that where open valences exist on these substituents they can be further substituted with alkyl, cycloalkyl, aryl, heteroaryl, and/or heterocycle groups, that where these open valences exist on carbon they can be further substituted by halogen and by oxygen-, nitrogen-, or sulfur-bonded substituents, and where multiple such open valences exist, these groups can be joined to form a ring, either by direct formation of a bond or by formation of bonds to a new heteroatom, preferably oxygen, nitrogen, or sulfur. It is further understood that the above substitutions can be made provided that replacing the hydrogen with the substituent does not introduce unacceptable instability to the molecules of the present invention, and is otherwise chemically reasonable.

The term “heteroatom-containing substituent” refers to substituents containing at least one non-halogen heteroatom. Examples of such substituents include, but are not limited to, hydroxy, oxo, nitro, methoxy, ethoxy, alkoxy, substituted alkoxy, trifluoromethoxy, haloalkoxy, hydroxyalkyl, alkoxyalkyl, thio, alkylthio, acyl, carboxy, alkoxycarbonyl, carboxamido, substituted carboxamido, alkylsulfonyl, alkylsulfinyl, alkylsulfonylamino, sulfonamido, substituted sulfonamido, cyano, amino, substituted amino, alkylamino, dialkylamino, aminoalkyl, acylamino, amidino, amidoximo, hydroxamoyl, aryloxy, pyridyl, imidazolyl, heteroaryl, substituted heteroaryl, heteroaryloxy, heteroarylalkyl, heteroarylalkenyl, heteroarylalkynyl, cycloalkyloxy, pyrrolidinyl, piperidinyl, morpholino, heterocycle, (heterocycle)oxy, and (heterocycle)alkyl; and preferred heteroatoms are oxygen, nitrogen, and sulfur. It is understood that where open valences exist on these substituents they can be further substituted with alkyl, cycloalkyl, aryl, heteroaryl, and/or heterocycle groups, that where these open valences exist on carbon they can be further substituted by halogen and by oxygen-, nitrogen-, or sulfur-bonded substituents, and where multiple such open valences exist, these groups can be joined to form a ring, either by direct formation of a bond or by formation of bonds to a new heteroatom, preferably oxygen, nitrogen, or sulfur. It is further understood that the above substitutions can be made provided that replacing the hydrogen with the substituent does not introduce unacceptable instability to the molecules of the present invention, and is otherwise chemically reasonable.

“Pharmaceutically acceptable salts” are salts that retain the desired biological activity of the parent compound and do not impart undesired toxicological effects. Pharmaceutically acceptable salt forms include various polymorphs as well as the amorphous form of the different salts derived from acid or base additions. The acid addition salts can be formed with inorganic or organic acids, Illustrative but not restrictive examples of such acids include hydrochloric, hydrobromic, sulfuric, phosphoric, citric, acetic, propionic, benzoic, napthoic, oxalic, succinic, maleic, fumaric, malic, adipic, lactic, tartaric, salicylic, methanesulfonic, 2-hydroxyethanesulfonic, toluenesulfonic, benzenesulfonic, camphorsulfonic, and ethanesulfonic acids. The pharmaceutically acceptable base addition salts can be formed with metal or organic counterions and include, but are not limited to, alkali metal salts such as sodium or potassium; alkaline earth metal salts such as magnesium or calcium; and ammonium or tetraalkyl ammonium salts, i.e., NX₄⁺ (wherein X is C₁₋₄).

“Tautomers” are compounds that can exist in one or more forms, called tautomeric forms, which can interconvert by way of a migration of one or more hydrogen atoms in the compound accompanied by a rearrangement in the position of adjacent double bonds. These tautomeric forms are in equilibrium with each other, and the position of this equilibrium will depend on the exact nature of the physical state of the compound. It is understood that where tautomeric forms are possible, the current invention relates to all possible tautomeric forms.

“Solvates” are addition complexes in which a compound of Formula I or Formula II is combined with a pharmaceutically acceptable cosolvent in some fixed proportion. Cosolvents include, but are not limited to, water, methanol, ethanol, 1-propanol, isopropanol, 1-butanol, isobutanol, tert-butanol, acetone, methyl ethyl ketone, acetonitrile, ethyl acetate, benzene, toulene, xylene(s), ethylene glycol, dichloromethane, 1,2-dichloroethane, N-methylformamide, N,N-dimethylformamide, N-methylacetamide, pyridine, dioxane, and diethyl ether. Hydrates are solvates in which the cosolvent is water. It is to be understood that the definitions of compounds in Formula I and Formula II encompass all possible hydrates and solvates, in any proportion, which possess the stated activity.

The term “edema” refers to an abnormal accumulation of extra-vascular fluid.

The term “inflammation” generally refers to a localized reaction of tissue, characterized by the influx of immune cells, which occurs in reaction to injury or infection.

“An effective amount” is the amount effective to treat a disease by ameliorating the pathological condition or reducing the symptoms of the disease. “An effective amount” is the amount effective to improve at least one of the parameters relevant to measurement of the disease.

The inventors of the present invention have discovered that compounds of Formula I or II, which are Rho kinase inhibitors, are effective in reducing cell proliferation, decreasing remodeling that is defined by cell migration and/or proliferation, reducing inflammation via the inhibition of leukocytes chemotaxis and the inhibition of cytokine and chemokine secretion, lowering or preventing tissue or organ edema via the increase of endothelial cell junction integrity, and reducing vasoconstriction via the disruption of acto-myosin-based cytoskeleton within smooth muscle cells, thereby reducing smooth muscle tone and contractibility. By having the above properties, compounds of Formula I or II are useful in a method of preventing or treating inflammatory diseases.

The invention provides a method of reducing excessive cell proliferation, a method of decreasing remodeling that is defined by cell migration and/or proliferation, a method of reducing inflammation via inhibition of leukocytes chemotaxis and via decreasing cytokine and chemokine secretion, and a method of lowering or preventing tissue or organ edema via increasing endothelial and epithelial cell junction integrity. By resolving one or more of the above-described pathophysiologies, the present invention provides a method of treating of inflammatory diseases, particularly rheumatoid arthritis and inflammatory bowel disease.

The present method comprises the steps of identifying a subject in need of treatment for the above conditions, and administering to the subject an effective amount of a rho kinase inhibitor compound of Formula I or II.

### Rho Kinase Inhibitor Compounds

The rho kinase inhibitor compounds useful for this invention include compounds of general Formula I and Formula II, and/or tautomers thereof, and/or pharmaceutically-acceptable salts, and/or solvates, and/or hydrates thereof. Compounds of general Formula I and Formula II can be prepared according to the methods disclosed in co-pending application US2008/0214614, which is incorporated herein by reference.

A compound according to Formula I or Formula II can exist in several diastereomeric forms. The general structures of Formula I and Formula II include all diastereomeric forms of such materials, when not specified otherwise. Formula I and Formula II also include mixtures of compounds of these Formulae, including mixtures of enantiomers, diastereomers and/or other isomers in any proportion.

### A. Formula I

Compounds of Formula I are as follows:

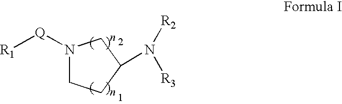

wherein: R₁ is aryl or heteroaryl, optionally substituted;
Q is C═O, SO₂, or (CR₄R₅)ₙ₃;
n₁ is 1, 2, or 3;
n₂ is 1 or 2;
n₃ is 0, 1, 2, or 3;
wherein the ring represented by

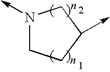

is optionally substituted by alkyl, halo, oxo, OR₆, NR₆R₇, or SR₆;
R₂ is selected from the following heteroaryl systems, optionally substituted:

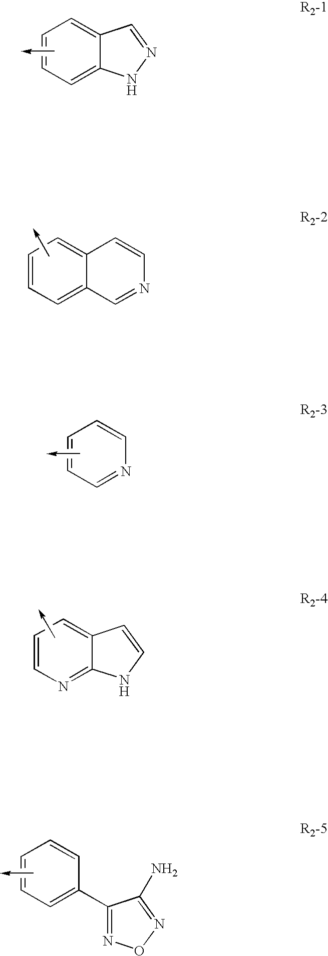

R₃-R₇ are independently H, alkyl, alkenyl, alkynyl, cycloalkyl, cycloalkenyl, cycloalkylalkyl, cycloalkylalkenyl, or cycloalkylalkynyl optionally substituted.

In Formula I, a preferred R₁ is substituted aryl, a more preferred R₁ is substituted phenyl, the preferred Q is (CR₄R₅)ₙ₃, the more preferred Q is CH₂, the preferred n₁ is 1 or 2, the preferred n₂ is 1, the preferred n₃ is 1 or 2, and the preferred R₃-R₇ are H.

In Formula I, a preferred R₂ substituent is halo, alkyl, cycloalkyl, hydroxyl, alkoxy, cycloalkyloxy, amino, alkylamino, or R₂ is unsubstituted. A more preferred R₂ substituent is halo, methyl, ethyl, isopropyl, cyclopropyl, hydroxyl, methoxy, ethoxy, amino, methylamino, dimethylamino, or R₂ is unsubstituted.

[1] One embodiment of the invention is represented by Formula I, in which R₂ is 5-indazolyl or 6-indazolyl (R₂-1), optionally substituted.

[1a] In embodiment 1, R₂-1 is substituted by one or more alkyl or halo substituents.

[1b] In embodiment 1, R₂-1 is substituted by one or more amino, alkylamino, hydroxyl, or alkoxy substituents.

[1c] In embodiment 1, R₂-1 is unsubstituted.

[2] In another embodiment, the invention is represented by Formula I in which R₂ is 5-isoquinolinyl or 6-isoquinolinyl (R₂-2), optionally substituted.

[2a] In embodiment 2, R₂-2 is substituted by one or more alkyl or halo substituents.

[2b] In embodiment 2, R₂-2 is substituted by one or more amino, alkylamino, hydroxyl, or alkoxy substituents.

[2c] In embodiment 2, R₂-2 is unsubstituted.

[3] In another embodiment, the invention is represented by Formula I in which R₂ is 4-pyridyl or 3-pyridyl (R₂-3), optionally substituted.

[3a] In embodiment 3, R₂-3 is substituted by one or more alkyl or halo substituents.

[3b] In embodiment 3, R₂-3 is substituted by one or more amino, alkylamino, hydroxyl, or alkoxy substituents.

[3c] In embodiment 3, R₂-3 is unsubstituted.

[4] In another embodiment, the invention is represented by Formula I in which R₂ is 7-azaindol-4-yl or 7-azaindol-5-yl (R₂-4), optionally substituted.

[4a] In embodiment 4, R₂-4 is substituted by one or more alkyl or halo substituents.

[4b] In embodiment 4, R₂-4 is substituted by one or more amino, alkylamino, hydroxyl, or alkoxy substituents.

[4c] In embodiment 4, R₂-4 is unsubstituted.

[5] In another embodiment, the invention is represented by Formula I in which R₂ is 4-(3-amino-1,2,5-oxadiazol-4-yl)phenyl or 3-(3-amino-1,2,5-oxadiazol-4-yl)phenyl (R₂-5), optionally substituted.

[5a] In embodiment 5, R₂-5 is unsubstituted.

[6] In another embodiment, the invention is represented by Formula I in which R₂ is one of the groups R₂-1-R₂-5, substituted by one or more alkyl, halo, amino, alkylamino, hydroxyl, or alkoxy substituents.

[6a] In embodiment 6, R₂ is substituted by one or more alkyl or halo substituents.

[6b] In embodiment 6, R₂ is substituted by one or more amino, alkylamino, hydroxyl, or alkoxy substituents.

[7] In another embodiment, the invention is represented by Formula I in which R₂ is one of the groups R₂-1-R₂-5, and is unsubstituted.

[8] In another embodiment, the invention is represented by Formula I in which R₃ is H.

[9] In another embodiment, the invention is represented by Formula I in which Q is (CR₄R₅)ₙ₃, and n₃ is 1 or 2.

[10] In another embodiment, the invention is represented by Formula I in which Q is (CH₂)ₙ₃, and n₃ is 1.

[11] In another embodiment, the invention is represented by Formula I in which R₁ is aryl or heteroaryl substituted with one or more alkenyl, alkynyl, aryl, arylalkyl, arylalkenyl, arylalkynyl, heteroaryl, heteroarylalkyl, heteroarylalkenyl, heteroarylalkynyl, cycloalkyl, cycloalkenyl, cycloalkylalkyl, cycloalkylalkenyl, cycloalkylalkynyl, heterocycle, (heterocycle)alkyl, (heterocycle)alkenyl, or (heterocycle)alkynyl substituents, optionally further substituted.

Compounds exemplifying embodiment 11 include compounds 1.009, 1.010, 1.011, 1.012, 1.020, 1.021, 1.030, 1.034, 1.037, 1.044, 1.047, 1.076, 1.077, 1.083, 2.010, 2.011, 2.019, 2.020, 2.022, 2.023, and 2.031, shown below in Table A.

[12] In another embodiment, the invention is represented by Formula I in which R₁ is aryl or heteroaryl substituted with one or more heteroatom-containing substituents, with the proviso that if the R₁ substituent is acyclic and is connected to R₁ by a carbon atom, then this substituent contains at least one nitrogen or sulfur atom, with the second proviso that if the substituent is acyclic and is connected to R₁ by an oxygen or nitrogen atom, then this substituent contains at least one additional oxygen, nitrogen or sulfur atom, and with the third proviso that if the substituent is connected to R₁ by a sulfone linkage “—SO₂-”, then R₂ is not nitrogen- or oxygen-substituted R₂-2.

[12a] In embodiment 12, the heteroatom-containing substituent is connected to R₁ by an oxygen or nitrogen atom.

[12b] In embodiment 12, the heteroatom-containing substituent is connected to R₁ by a sulfide linkage, “—S—”.

Compounds exemplifying embodiment 12 include compounds 1.001, 1.002, 1.004, 1.005, 1.038, 1.048, 1.055, 1.056, 2.002, 2.003, 2.005, 2.007, 1.003, 1.006, 1.007, 1.018, 1.039, 1.051, 1.058, 1.060, 1.084, 1.085, 1.086, 1.087, 1.088, 1.090, 1.091, 1.092, 1.093, 1.094, 1.095, 1.096, 1.097, 1.098, 1.102, 1.111, 1.113, 1.115, 1.116, 1.117, 1.118, 1.120, 1.121, 1.123, 1.124, 1.125, 1.126, 1.127, 1.128, 1.129, 1.130, 2.004, 2.008, 2.032, 2.033, 2.034, 2.035, 2.036, 2.037, 2.038, 2.039, 2.040, 2.041, 2.042, 2.043, 2.044, 1.008, 1.017, 1.026, 1.040, 1.074, 1.075, 2.009, 2.012, 2.021, 2.024, 2.026, and 2.029, shown below in Table A.

[13] In another embodiment, the invention is represented by Formula I in which R₁ is aryl or heteroaryl substituted with one or more alkyl, alkenyl, alkynyl, aryl, arylalkyl, arylalkenyl, arylalkynyl, heteroaryl, heteroarylalkyl, heteroarylalkenyl, heteroarylalkynyl, cycloalkyl, cycloalkenyl, cycloalkylalkyl, cycloalkylalkenyl, cycloalkylalkynyl, heterocycle, (heterocycle)alkyl, (heterocycle)alkenyl, or (heterocycle)alkynyl substituents, which are further substituted with one or more heteroatom-containing substituents, with the proviso that if the R₁ substituent is acyclic and its heteroatom-containing substituent falls on the carbon by which it is attached to R₁, then the heteroatom-containing substituent contains at least one nitrogen or sulfur atom.

Compounds exemplifying embodiment 13 include compounds 1.019, 1.027, 1.028, 1.029, 1.035, 1.041, 1.042, 1.043, 1.057, 1.061, 1.099, 1.101, 1.103, 1.104, 1.105, 1.106, 1.107, 1.108, 1.109, 1.112, 1.114, 1.119, 1.122, and 1.123, shown below in Table A.

[14] In another embodiment, the invention is represented by Formula I in which R₁ is aryl or heteroaryl substituted with one or more alkenyl, alkynyl, aryl, arylalkyl, arylalkenyl, arylalkynyl, heteroaryl, heteroarylalkyl, heteroarylalkenyl, heteroarylalkynyl, cycloalkyl, cycloalkenyl, cycloalkylalkyl, cycloalkylalkenyl, cycloalkylalkynyl, heterocycle, (heterocycle)alkyl, (heterocycle)alkenyl, or (heterocycle)alkynyl substituents, optionally further substituted, and R₂ is 5-indazolyl (R₂-1) or 5-isoquinolinyl (R₂-2), optionally substituted.

[14a] In embodiment 14, R₂ is 5-indazolyl (R₂-1), optionally substituted by one or more alkyl, halo, amino, alkylamino, hydroxyl, or alkoxy substituents.

[14b] In embodiment 14, R₂ is 5-isoquinolinyl (R₂-2), optionally substituted by one or more alkyl, halo, amino, alkylamino, hydroxyl, or alkoxy substituents.

[14c] In embodiment 14, R₂ is unsubstituted. Compounds exemplifying embodiment 14 include compounds 1.009, 1.010, 1.011, 1.012, 1.020, 1.021, 1.030, 1.034, 1.037, 1.044, 1.047, 1.076, 1.077, 1.083, 2.010, 2.011, 2.019, 2.020, 2.022, 2.023, and 2.031, shown below in Table A.

[15] In another embodiment, the invention is represented by Formula I in which R₁ is aryl or heteroaryl substituted with one or more heteroatom-containing substituents, and R₂ is 5-indazolyl (R₂-1) or 5-isoquinolinyl (R₂-2), optionally substituted, with the proviso that if the R₁ substituent is acyclic and is connected to R₁ by a carbon atom, then this substituent contains at least one nitrogen or sulfur atom, with the second proviso that if the substituent is acyclic and is connected to R₁ by an oxygen or nitrogen atom, then this substituent contains at least one additional oxygen, nitrogen or sulfur atom, and with the third proviso that if the substituent is connected to R₁ by a sulfone linkage “—SO₂—”, then R₂ is not nitrogen- or oxygen-substituted R₂-2.

[15a] In embodiment 15, R₂ is 5-indazolyl (R₂-1), optionally substituted by one or more alkyl, halo, amino, alkylamino, hydroxyl, or alkoxy substituents.

[15b] In embodiment 15, R₂ is 5-isoquinolinyl (R₂-2), optionally substituted by one or more alkyl, halo, amino, alkylamino, hydroxyl, or alkoxy substituents.

[15c] In embodiment 15, R₂ is unsubstituted.

[15d] In embodiment 15, the heteroatom-containing substituent is connected to R₁ by an oxygen or nitrogen atom.

[15e] In embodiment 15, the heteroatom-containing substituent is connected to R₁ by a sulfide linkage, “—S—”.

Compounds exemplifying embodiment 15 include compounds 1.001, 1.002, 1.004, 1.005, 1.038, 1.048, 1.055, 1.056, 2.002, 2.003, 2.005, 2.007, 1.003, 1.006, 1.007, 1.018, 1.039, 1.051, 1.058, 1.060, 1.084, 1.085, 1.086, 1.087, 1.088, 1.090, 1.091, 1.092, 1.093, 1.094, 1.095, 1.096, 1.097, 1.098, 1.102, 1.111, 1.113, 1.115, 1.116, 1.117, 1.118, 1.120, 1.121, 1.123, 1.124, 1.125, 1.126, 1.127, 1.128, 1.129, 1.130, 2.004, 2.008, 2.032, 2.033, 2.034, 2.035, 2.036, 2.037, 2.038, 2.039, 2.040, 2.041, 2.042, 2.043, 2.044, 1.008, 1.017, 1.026, 1.040, 1.074, 1.075, 2.009, 2.012, 2.021, 2.024, 2.026, and 2.029, shown below in Table A.

[16] In another embodiment, the invention is represented by Formula I in which R₁ is aryl or heteroaryl substituted with one or more alkyl, alkenyl, alkynyl, aryl, arylalkyl, arylalkenyl, arylalkynyl, heteroaryl, heteroarylalkyl, heteroarylalkenyl, heteroarylalkynyl, cycloalkyl, cycloalkenyl, cycloalkylalkyl, cycloalkylalkenyl, cycloalkylalkynyl, heterocycle, (heterocycle)alkyl, (heterocycle)alkenyl, or (heterocycle)alkynyl substituents, at least one of which is further substituted with one or more heteroatom-containing substituents, and R₂ is 5-indazolyl (R₂-1) or 5-isoquinolinyl (R₂-2), optionally substituted, with the proviso that if the R₁ substituent is acyclic and its heteroatom-containing substituent falls on the carbon by which it is attached to R₁, then the heteroatom-containing substituent contains at least one nitrogen or sulfur atom.

[16a] In embodiment 16, R₂ is 5-indazolyl (R₂-1), optionally substituted by one or more alkyl, halo, amino, alkylamino, hydroxyl, or alkoxy substituents.

[16b] In embodiment 16, R₂ is 5-isoquinolinyl (R₂-2), optionally substituted by one or more alkyl, halo, amino, alkylamino, hydroxyl, or alkoxy substituents.

[16c] In embodiment 16, R₂ is unsubstituted.

Compounds exemplifying embodiment 16 include compounds 1.019, 1.027, 1.028, 1.029, 1.035, 1.041, 1.042, 1.043, 1.057, 1.061, 1.099, 1.101, 1.103, 1.104, 1.105, 1.106, 1.107, 1.108, 1.109, 1.112, 1.114, 1.119, 1.122, and 1.123, shown below in Table A.

The inventors have discovered certain compounds of Formula I that have properties that render them particularly useful for treating the conditions addressed by the invention. In particular, these preferred compounds can be described as compounds of Formula I in which R₂, R₃, n₁, and n₂ are limited to the combinations shown in Formulae Ia, Ib, and Ic:

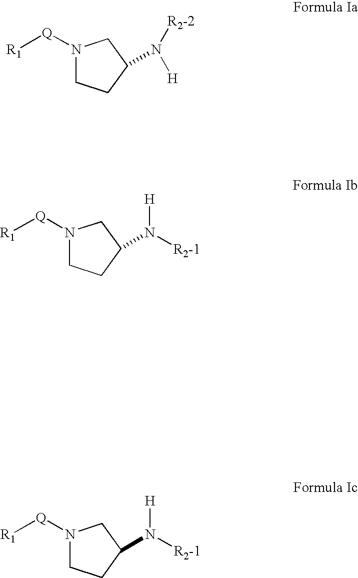

In Formulae Ia, Ib, and Ic, the stereochemistry of the central pyrrolidine or piperidine ring is limited to the R, R, and S configurations respectively, as drawn. Further, the group R₁ in these Formulae is limited to phenyl, thiophene, and 6,5- or 6,6-fused bicyclic heteroaryl rings. The group R₁ is either unsubstituted or is optionally substituted with 1, 2 or 3 substituents independently selected from halogen, methyl, ethyl, hydroxyl, methoxy, or ethoxy.

In Formula Ia, Ib, and Ic, Q is C═O, SO₂, or (CR₄R₅)ₙ₃; where R₄ and R₅ are independently H, alkyl, cycloalkyl, optionally substituted. The preferred R₄ and R₅ are H or unsubstituted alkyl. The preferred Q is CH₂.

In Formula Ia, Ib, and Ic, a preferred R₂ substituent is halo, alkyl, cycloalkyl, hydroxyl, alkoxy, cycloalkyloxy, amino, alkylamino, or R₂ is unsubstituted. A more preferred R₂ substituent is halo, methyl, ethyl, isopropyl, cyclopropyl, hydroxyl, methoxy, ethoxy, amino, methylamino, dimethylamino, or R₂ is unsubstituted.

In a more preferred form of Formulae Ia, Ib, and Ic, R₁ is phenyl or a 6,5-fused bicyclic heteroaryl ring, optionally substituted by 1 or 2 substituents, Q is CH₂, and the group R₂ is unsubstituted. The most preferred 6,5-fused bicyclic heteroaryl rings are benzofuran, benzothiophene, indole, and benzimidazole.

In another more preferred form, R₁ of Formulae Ia, Ib, and Ic is mono- or disubstituted when R₁ is phenyl, with 3-substituted, 4-substituted, 2,3-disubstituted, and 3,4-disubstituted being most preferred. When R₁ is bicyclic heteroaryl, an unsubstituted or monosubstituted R₁ is most preferred.

The inventors have found that certain members of Formulae Ia, Ib, and Ic, as defined above, are particularly useful in treating the conditions addressed in this invention. The compounds of the invention are multikinase inhibitors, with inhibitory activity against ROCK1 and ROCK2, in addition to several other kinases in individual compound cases. These kinase inhibitory properties endow the compounds of the invention not only with smooth muscle relaxant properties, but additionally with antiproliferative, antichemotactic, and cytokine secretion inhibitory properties that render them particularly useful in treating conditions with proliferative or inflammatory components as described in the invention.

[17] In particular, we have found that compounds in which R₂ is R₂-2 are particularly potent inhibitors of both ROCK1 and ROCK2, and that these agents inhibit the migration of neutrophils toward multiple chemotactic stimuli and inhibit the secretion of the cytokines IL-1β, TNF-α and IL-9 from LPS-stimulated human monocytes. Compounds in which R₁ is heteroaryl, particularly 6,5-fused bicyclic heteroaryl, are especially preferred. These compounds are of particular value in addressing conditions with an inflammatory component.

Compounds exemplifying embodiment 17 include compounds 2.025, 2.027, 2.046, 2.047, 2.048, 2.055, 2.056, 2.057, 2.061, 2.062, 2.065, 2.074, 2.075, 2.088, and 2.090.

[18] In another embodiment, we have found that compounds of Formula Ic are potent and selective inhibitors of ROCK2, with comparatively lower inhibitory potency against ROCK1. We have demonstrated that compounds of this class typically show good smooth muscle relaxation properties and that smooth muscle relaxation effects in this class are generally correlated with ROCK2 potency. Compounds in which R₁ is phenyl are particularly preferred. Compounds of this embodiment are of particular value in addressing conditions where relaxation of smooth muscle, in particular vascular and bronchial smooth muscle, is of highest importance.

Compounds exemplifying embodiment 18 include compounds 1.072, 1.078, 1.079, 1.080, 1.141, 1.142, 1.148, 1.149, 1.150, 1.151, 1.154, 1.155, 1.156, 1.163, 1.164, 1.166, 1.170, 1.171, 1.175, 1.179, 1.183, 1.227, 1.277, and 1.278.

[19] In another embodiment, the inventors have found that compounds of Formula Ib are potent mixed inhibitors of ROCK1 and ROCK2, display additional inhibitory activity against the kinases Akt3 and p70S6K, and that these compounds generally display potent antiproliferative activity in models of smooth muscle cell proliferation. Compounds of this class are of particular value in addressing conditions in which an antiproliferative component is desired in combination with a smooth muscle relaxing effect.

Compounds exemplifying embodiment 19 include compounds 1.073, 1.110, 1.131, 1.132, 1.133, 1.134, 1.135, 1.136, 1.137, 1.138, 1.143, 1.144, 1.145, 1.146, 1.172, 1.173, 1.177, 1.191, 1.192, 1.203, 1.210, 1.226, 1.241, 1.242, 1.245, 1.246, 1.252, and 1.254.

[20] In another embodiment, the inventors have found that certain compounds of Formulae Ia, Ib, and Ic distribute preferentially to the lung on oral dosing. In particular, compounds in which R₁ is a lipophilic bicyclic heteroaryl group are preferred for this dosing behavior. Compounds of this type are especially useful for treating diseases of the lung by oral dosing while minimizing impact on other tissues.

Compounds exemplifying embodiment 20 include compounds 1.131, 1.137, 1.138, 1.143, 1.148, 1.149, 1.150, 1.166, 1.175, 1.177, 1.246, 1.252, 2.055, 2.056, 2.057, 2.065, 2.074, and 2.075.

[21] In another embodiment, the inventors have found that certain compounds of Formulae Ia, Ib, and Ic produce low plasma concentrations of the compound when dosed by the oral route. Compounds in which one substituent on R₁ is selected from the group methyl, ethyl, or hydroxyl are preferred for typically exhibiting this pharmacokinetic behavior. Compounds displaying this property are particularly useful for inhalation dosing, since a large portion of the material dosed in this way is typically swallowed, and it is advantageous for this swallowed portion to remain unabsorbed or to be cleared rapidly so as to minimize the impact of the compound on other tissues.

Compounds exemplifying embodiment 21 include compounds 1.078, 1.133, 1.135, 1.136, 1.145, 1.151, 1.154, 1.155, 1.156, 1.163, 1.171, 1.172, 1.173, 1.192, 1.242, 2.025, and 2.061.

Preparation of compounds of Formulae Ia, Ib, and Ic can be problematic using methods commonly known in the art. In particular, syntheses of compounds of Formulae Ib and Ic using transition metal mediated coupling reactions to form the critical bond between R₂-1 and the nitrogen atom are hampered by low yields when the indazole ring is not protected properly to allow a successful reaction. Specifically, the methods disclosed in UA2006/0167043 fail to provide the desired amino indazole products when the indazole is unprotected or is protected with a standard acyl protecting group such as pivalate or alkoxycarbonyl protecting groups. The inventors prepare compounds of Formulae Ia, Ib, and Ic according to the methods disclosed in the co-pending application US2008/0214614, which allows the successful protection, coupling, and deprotection of the indazole ring, thereby allowing the successful preparation of the compounds of Formulae Ib and Ic and the demonstration of their useful biological properties.

### B. Formula II

A preferred compound of Formula I is where R₁=Ar—X, shown below as Formula II:

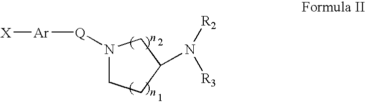

wherein:
Ar is a monocyclic or bicyclic aryl or heteroaryl ring, such as phenyl;
X is from 1 to 3 substituents on Ar, each independently in the form Y-Z, in which Z is attached to Ar;
Y is one or more substituents on Z, and each is chosen independently from H, halogen, or the heteroatom-containing substituents, including but not limited to OR₈, NR₈R₉, NO₂, SR₈, SOR₈, SO₂R₈, SO₂NR₈R₉, NR₈SO₂R₉, OCF₃, CONR₈R₉, NR₈C(═O)R₉, NR₈C(═O)OR₉, OC(═O)NR₈R₉, or NR₈C(═O)NR₉R₁₀;

Each instance of Z is chosen independently from alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, heterocycle, (heterocycle)alkyl, (heterocycle)alkenyl, (heterocycle)alkynyl, or is absent;

R₈ is H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle; optionally substituted by one or more halogen or heteroatom-containing substituents, including but not limited to OR₁₁, NR₁₁R₁₂, NO₂, SR₁₁, SOR₁₁, SO₂R₁₁, SO₂NR₁₁R₁₂, NR₁₁SO₂R₁₂, OCF₃, CONR₁₁R₁₂, NR₁₁C(═O)R₁₂, NR₁₁C(═O)OR₁₂, OC(═O)NR₁₁R₁₂, or NR₁₁C(═O)NR₁₂R₁₃;
R₉ and R₁₀ are independently H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle; optionally substituted by one or more halogen or heteroatom-containing substituents, including but not limited to OR₁₄, NR₁₄R₁₅, NO₂, SR₁₄, SOR₁₄, SO₂R₁₄, SO₂NR₁₄R₁₅, NR₁₄SO₂R₁₅, OCF₃, CONR₁₄R₁₅, NR₁₄C(═O)R₁₅, NR₁₄C(═O)OR₁₅, OC(═O)NR₁₄R₁₅, or NR₁₄C(═O)NR₁₅R₁₆;
any two of the groups R₈, R₉ and R₁₀ are optionally joined with a link selected from the group consisting of bond, —O—, —S—, —SO—, —SO₂—, and —NR₁₇— to form a ring;
R₁₁-R₁₇ are independently H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle.

In Formula II, the preferred Y is H, halogen, OR₈, NR₈R₉, NO₂, SR₈, SOR₈, SO₂R₈, SO₂NR₈R₉, NR₈SO₂R₉, OCF₃, CONR₈R₉, NR₈C(═O)R₉, NR₈C(═O)OR₉, OC(═O)NR₈R₉, or NR₈C(═O)NR₉R₁₀, the more preferred Y is H, halogen, OR₈, SR₈, SOR₈, SO₂R₈, SO₂NR₈R₉, NR₈SO₂R₉, CONR₈R₉, or NR₈C(═O)NR₉R₁₀, the preferred Z is alkyl, alkenyl, alkynyl, cycloalkyl, cycloalkenyl, cycloalkylalkyl, or is absent; the more preferred Z is alkyl, alkenyl, alkynyl, cycloalkyl, or is absent, the preferred Q is (CR₄R₅)ₙ₃, the more preferred Q is CH₂, the preferred n₁ is 1 or 2, the preferred n₂ is 1, the preferred n₃ is 1 or 2, the preferred R₃-R₇ are H, the preferred R₈ is H, alkyl, arylalkyl, cycloalkyl, cycloalkylalkyl, or heterocycle, the preferred R₈ substituents are H, halogen, OR₁₁, NR₁₁R₁₂, SR₁₁, SOR₁₁, SO₂R₁₁, SO₂NR₁₁R₁₂, NR₁₁SO₂R₁₂, CONR₁₁R₁₂, NR₁₁C(═O)R₁₂, and the preferred R₉-R₁₇ are H or alkyl.

In Formula II, a preferred R₂ substituent is halo, alkyl, cycloalkyl, hydroxyl, alkoxy, cycloalkyloxy, amino, alkylamino, or R₂ is unsubstituted. A more preferred R₂ substituent is halo, methyl, ethyl, isopropyl, cyclopropyl, hydroxyl, methoxy, ethoxy, amino, methylamino, dimethylamino, or R₂ is unsubstituted.

[1] One embodiment of the invention is represented by Formula II in which R₂ is 5-indazolyl or 6-indazolyl (R₂-1), optionally substituted.

[1a] In embodiment 1, R₂-1 is substituted by one or more alkyl or halo substituents.

[1b] In embodiment 1, R₂-1 is substituted by one or more amino, alkylamino, hydroxyl, or alkoxy substituents.

[1c] In embodiment 1, R₂-1 is unsubstituted.

[2] In another embodiment, the invention is represented by Formula II in which R₂ is 5-isoquinolinyl or 6-isoquinolinyl (R₂-2), optionally substituted.

[2a] In embodiment 2, R₂-2 is substituted by one or more alkyl or halo substituents.

[2b] In embodiment 2, R₂-2 is substituted by one or more amino, alkylamino, hydroxyl, or alkoxy substituents.

[2c] In embodiment 2, R₂-2 is unsubstituted.

[3] In another embodiment, the invention is represented by Formula II in which R₂ is 4-pyridyl or 3-pyridyl (R₂-3), optionally substituted.

[3a] In embodiment 3, R₂-3 is substituted by one or more alkyl or halo substituents.

[3b] In embodiment 3, R₂-3 is substituted by one or more amino, alkylamino, hydroxyl, or alkoxy substituents.

[3c] In embodiment 3, R₂-3 is unsubstituted.

[4] In another embodiment, the invention is represented by Formula II in which R₂ is 7-azaindol-4-yl or 7-azaindol-5-yl (R₂-4), optionally substituted.

[4a] In embodiment 4, R₂-4 is substituted by one or more alkyl or halo substituents.

[4b] In embodiment 4, R₂-4 is substituted by one or more amino, alkylamino, hydroxyl, or alkoxy substituents.

[4c] In embodiment 4, R₂-4 is unsubstituted.

[5] In another embodiment, the invention is represented by Formula II in which R₂ is 4-(3-amino-1,2,5-oxadiazol-4-yl)phenyl or 3-(3-amino-1,2,5-oxadiazol-4-yl)phenyl (R₂-5), optionally substituted.

[5a] In embodiment 5, R₂-5 is unsubstituted.

[6] In another embodiment, the invention is represented by Formula II in which R₂ is one of the groups R₂-1-R₂-5, substituted by one or more alkyl, halo, amino, alkylamino, hydroxyl, or alkoxy substituents.

[6a] In embodiment 6, R₂ is substituted by one or more alkyl or halo substituents.

[6b] In embodiment 6, R₂ is substituted by one or more amino, alkylamino, hydroxyl, or alkoxy substituents.

[7] In another embodiment, the invention is represented by Formula II in which R₂ is one of the groups R₂-1-R₂-5, and is unsubstituted.

[8] In another embodiment, the invention is represented by Formula II in which R₃ is H.

[9] In another embodiment, the invention is represented by Formula II in which Q is (CR₄R₅)ₙ₃, and n₃ is 1 or 2.

[10] In another embodiment, the invention is represented by Formula II in which Q is (CH₂)ₙ₃, and n₃ is 1.

[11] In another embodiment, the invention is represented by Formula II in which for at least one substituent X, Z is alkenyl, alkynyl, aryl, arylalkyl, arylalkenyl, arylalkynyl, heteroaryl, heteroarylalkyl, heteroarylalkenyl, heteroarylalkynyl, cycloalkyl, cycloalkylalkenyl, cycloalkylalkynyl, cycloalkenyl, cycloalkylalkyl, heterocycle, (heterocycle)alkyl, (heterocycle)alkenyl, or (heterocycle)alkynyl.

Compounds exemplifying embodiment 11 include compounds 1.009, 1.010, 1.011, 1.012, 1.020, 1.021, 1.030, 1.034, 1.037, 1.044, 1.047, 1.076, 1.077, 1.083, 2.010, 2.011, 2.019, 2.020, 2.022, 2.023, and 2.031, shown below in Table A.

[12] In another embodiment, the invention is represented by Formula II in which for at least one substituent X, Z is absent, and Y is a heteroatom-containing substituent, including but not limited to OR₈, NR₈R₉, SR₈, SOR₈, SO₂R₈, SO₂NR₈R₉, NR₈SO₂R₉, CONR₈R₉, NR₈C(═O)R₉, NR₈C(═O)OR₉, OC(═O)NR₈R₉, or NR₈C(═O)NR₉R₁₀, with the proviso that if the substituent Y is acyclic and is connected to Ar by a carbon atom, then this substituent contains at least one nitrogen or sulfur atom, with the second proviso that if the substituent Y is acyclic and is connected to Ar by an oxygen or nitrogen atom, then this substituent contains at least one additional oxygen, nitrogen or sulfur atom, and with the third proviso that if the substituent Y is connected to Ar by a sulfone linkage “—SO₂—”, then R₂ is not nitrogen- or oxygen-substituted R₂-2.

[12a] In embodiment 12, the heteroatom-containing substituent is connected to R₁ by an oxygen or nitrogen atom.

[12b] In embodiment 12, the heteroatom-containing substituent is connected to R₁ by a sulfide linkage, “—S—”.

Compounds exemplifying embodiment 12 include compounds 1.001, 1.002, 1.004, 1.005, 1.038, 1.048, 1.055, 1.056, 2.002, 2.003, 2.005, 2.007, 1.003, 1.006, 1.007, 1.018, 1.039, 1.051, 1.058, 1.060, 1.084, 1.085, 1.086, 1.087, 1.088, 1.090, 1.091, 1.092, 1.093, 1.094, 1.095, 1.096, 1.097, 1.098, 1.102, 1.111, 1.113, 1.115, 1.116, 1.117, 1.118, 1.120, 1.121, 1.123, 1.124, 1.125, 1.126, 1.127, 1.128, 1.129, 1.130, 2.004, 2.008, 2.032, 2.033, 2.034, 2.035, 2.036, 2.037, 2.038, 2.039, 2.040, 2.041, 2.042, 2.043, 2.044, 1.008, 1.017, 1.026, 1.040, 1.074, 1.075, 2.009, 2.012, 2.021, 2.024, 2.026, and 2.029, shown below in Table A.

[13] In another embodiment, the invention is represented by Formula II in which for at least one substituent X, Z is alkyl, alkenyl, alkynyl, aryl, arylalkyl, arylalkenyl, arylalkynyl, heteroaryl, heteroarylalkyl, heteroarylalkenyl, heteroarylalkynyl, cycloalkyl, cycloalkenyl, cycloalkylalkyl, cycloalkylalkenyl, cycloalkylalkynyl, heterocycle, (heterocycle)alkyl, (heterocycle)alkenyl, or (heterocycle)alkynyl, and Y is a heteroatom-containing substituent, including but not limited to OR₈, NR₈R₉, NO₂, SR₈, SOR₈, SO₂R₈, SO₂NR₈R₉, NR₈SO₂R₉, OCF₃, CONR₈R₉, NR₈C(═O)R₉, NR₈C(═O)OR₉, OC(═O)NR₈R₉, or NR₈C(═O)NR₉R₁₀, with the proviso that if Z is acyclic and Y falls on the carbon by which Z is attached to Ar, then Y contains at least one nitrogen or sulfur atom.

Compounds exemplifying embodiment 13 include compounds 1.019, 1.027, 1.028, 1.029, 1.035, 1.041, 1.042, 1.043, 1.057, 1.061, 1.099, 1.101, 1.103, 1.104, 1.105, 1.106, 1.107, 1.108, 1.109, 1.112, 1.114, 1.119, 1.122, and 1.123, shown below in Table A.

[14] In another embodiment, the invention is represented by Formula II in which for at least one substituent X, Z is alkenyl, alkynyl, aryl, arylalkyl, arylalkenyl, arylalkynyl, heteroaryl, heteroarylalkyl, heteroarylalkenyl, heteroarylalkynyl, cycloalkyl, cycloalkenyl, cycloalkylalkyl, cycloalkylalkenyl, cycloalkylalkynyl, heterocycle, (heterocycle)alkyl, (heterocycle)alkenyl, or (heterocycle)alkynyl, and R₂ is 5-indazolyl (R₂-1) or 5-isoquinolinyl (R₂-2), optionally substituted.

[14a] In embodiment 14, R₂ is 5-indazolyl (R₂-1), optionally substituted by one or more alkyl, halo, amino, alkylamino, hydroxyl, or alkoxy substituents.

[14b] In embodiment 14, R₂ is 5-isoquinolinyl (R₂-2), optionally substituted by one or more alkyl, halo, amino, alkylamino, hydroxyl, or alkoxy substituents.

[14c] In embodiment 14, R₂ is unsubstituted.

Compounds exemplifying embodiment 14 include compounds 1.009, 1.010, 1.011, 1.012, 1.020, 1.021, 1.030, 1.034, 1.037, 1.044, 1.047, 1.076, 1.077, 1.083, 2.010, 2.011, 2.019, 2.020, 2.022, 2.023, and 2.031, shown below in Table A.

[15] In another embodiment, the invention is represented by Formula II in which for at least one substituent X, Z is absent, and Y is a heteroatom-containing substituent, including but not limited to OR₈, NR₈R₉, SR₈, SOR₈, SO₂R₈, SO₂NR₈R₉, NR₈SO₂R₉, CONR₈R₉, NR₈C(═O)R₉, NR₈C(═O)OR₉, OC(═O)NR₈R₉, or NR₈C(═O)NR₉R₁₀, and R₂ is 5-indazolyl (R₂-1) or 5-isoquinolinyl (R₂-2), optionally substituted, with the proviso that if the substituent Y is acyclic and is connected to Ar by a carbon atom, then this substituent contains at least one nitrogen or sulfur atom, with the second proviso that if the substituent Y is acyclic and is connected to Ar by an oxygen or nitrogen atom, then this substituent contains at least one additional oxygen, nitrogen or sulfur atom, and with the third proviso that if the substituent Y is connected to Ar by a sulfone linkage “—SO₂—”, then R₂ is not nitrogen- or oxygen-substituted R₂-2.

[15a] In embodiment 15, R₂ is 5-indazolyl (R₂-1), optionally substituted by one or more alkyl, halo, amino, alkylamino, hydroxyl, or alkoxy substituents.

[15b] In embodiment 15, R₂ is 5-isoquinolinyl (R₂-2), optionally substituted by one or more alkyl, halo, amino, alkylamino, hydroxyl, or alkoxy substituents.

[15c] In embodiment 15, R₂ is unsubstituted.

[15d] In embodiment 15, the heteroatom-containing substituent is connected to R₁ by an oxygen or nitrogen atom.

[15e] In embodiment 15, the heteroatom-containing substituent is connected to R₁ by a sulfide linkage, “—S—”.

Compounds exemplifying embodiment 15 include compounds 1.001, 1.002, 1.004, 1.005, 1.038, 1.048, 1.055, 1.056, 2.002, 2.003, 2.005, 2.007, 1.003, 1.006, 1.007, 1.018, 1.039, 1.051, 1.058, 1.060, 1.084, 1.085, 1.086, 1.087, 1.088, 1.090, 1.091, 1.092, 1.093, 1.094, 1.095, 1.096, 1.097, 1.098, 1.102, 1.111, 1.113, 1.115, 1.116, 1.117, 1.118, 1.120, 1.121, 1.123, 1.124, 1.125, 1.126, 1.127, 1.128, 1.129, 1.130, 2.004, 2.008, 2.032, 2.033, 2.034, 2.035, 2.036, 2.037, 2.038, 2.039, 2.040, 2.041, 2.042, 2.043, 2.044, 1.008, 1.017, 1.026, 1.040, 1.074, 1.075, 2.009, 2.012, 2.021, 2.024, 2.026, and 2.029, shown below in Table A.

[16] In another embodiment, the invention is represented by Formula II in which for at least one substituent X, Z is alkyl, alkenyl, alkynyl, aryl, arylalkyl, arylalkenyl, arylalkynyl, heteroaryl, heteroarylalkyl, heteroarylalkenyl, heteroarylalkynyl, cycloalkyl, cycloalkenyl, cycloalkylalkyl, cycloalkylalkenyl, cycloalkylalkynyl, heterocycle, (heterocycle)alkyl, (heterocycle)alkenyl, or (heterocycle)alkynyl, and Y is a heteroatom-containing substituent, including but not limited to OR₈, NR₈R₉, NO₂, SR₈, SOR₈, SO₂R₈, SO₂NR₈R₉, NR₈SO₂R₉, OCF₃, CONR₈R₉, NR₈C(═O)R₉, NR₈C(═O)OR₉, OC(═O)NR₈R₉, or NR₈C(═O)NR₉R₁₀, and R₂ is 5-indazolyl (R₂-1) or 5-isoquinolinyl (R₂-2), optionally substituted, with the proviso that if Z is acyclic and Y falls on the carbon by which Z is attached to Ar, then Y contains at least one nitrogen or sulfur atom.

[16a] In embodiment 16, R₂ is 5-indazolyl (R₂-1), optionally substituted by one or more alkyl, halo, amino, alkylamino, hydroxyl, or alkoxy substituents.

[16b] In embodiment 16, R₂ is 5-isoquinolinyl (R₂-2), optionally substituted by one or more alkyl, halo, amino, alkylamino, hydroxyl, or alkoxy substituents.

[16c] In embodiment 16, R₂ is unsubstituted.

[16d] In embodiment 16, Ar is heteroaryl. Compounds exemplifying embodiment 16 include compounds 1.019, 1.027, 1.028, 1.029, 1.035, 1.041, 1.042, 1.043, 1.057, 1.061, 1.099, 1.101, 1.103, 1.104, 1.105, 1.106, 1.107, 1.108, 1.109, 1.112, 1.114, 1.119, 1.122, and 1.123, shown below in Table A.

In Embodiments 11-16 of Formula II, the preferred Q is (CR₄R₅)ₙ₃, the more preferred Q is CH₂, the preferred n1 is 1 or 2, the preferred n₂ is 1, the preferred n₃ is 1 or 2, and the preferred R₃ is H.

The inventors have discovered certain compounds of Formula II that have properties that render them particularly useful for treating the conditions addressed by the invention. In particular, these preferred compounds of Embodiments 14, 15 and 16 can be described as compounds of Formula II in which R₂, R₃, n₁, and n₂ are limited to the combinations shown in Formulae IIa, IIb, and IIc:

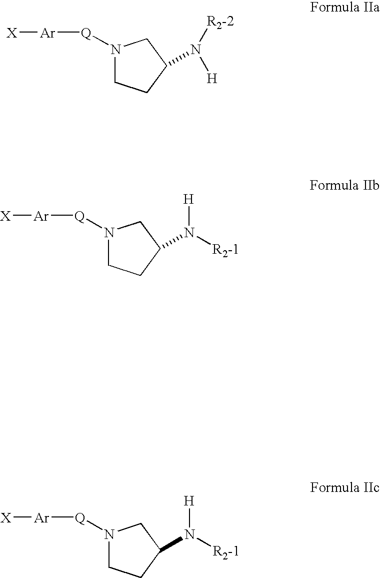

In Formulae IIa, IIb, and IIc, the stereochemistry of the central pyrrolidine or piperidine ring is limited to the R, R, and S configurations respectively, as drawn.

In Formula IIa, IIb, and IIc, Q is C═O, SO₂, or (CR₄R₅)ₙ₃; where R₄ and R₅ are independently H, alkyl, cycloalkyl, optionally substituted. The preferred R₄ and R₅ are H or unsubstituted alkyl. The preferred Q is CH₂.

In Formula IIa, IIb, and IIc, a preferred R₂ substituent is halo, alkyl, cycloalkyl, hydroxyl, alkoxy, cycloalkyloxy, amino, alkylamino, or R₂ is unsubstituted. A more preferred R₂ substituent is halo, methyl, ethyl, isopropyl, cyclopropyl, hydroxyl, methoxy, ethoxy, amino, methylamino, dimethylamino, or R₂ is unsubstituted.

In a more preferred form of Formulae IIa, IIb, and IIc, Ar is phenyl or a 6,5- or 6,6-fused bicyclic heteroaryl ring, substituted by 1 or 2 substituents X, and Q is CH₂. The most preferred 6,5-fused bicyclic heteroaryl rings are benzofuran, benzothiophene, indole, and benzimidazole.

In its more preferred form, Ar of Formulae IIa, IIb, and IIc is mono- or disubstituted when Ar is phenyl, with 3-substituted, 4-substituted, 2,3-disubstituted, and 3,4-disubstituted being most preferred. When Ar is bicyclic heteroaryl, a monosubstituted Ar is most preferred.

The inventors have found that certain members of Formulae IIa, IIb, and IIc, as defined above, are particularly useful in treating the conditions addressed in this invention. The compounds of the invention are multikinase inhibitors, with inhibitory activity against ROCK1 and ROCK2, in addition to several other kinases in individual compound cases. These kinase inhibitory properties endow the compounds of the invention not only with smooth muscle relaxant properties, but additionally with antiproliferative, antichemotactic, and cytokine secretion inhibitory properties that render them particularly useful in treating conditions with proliferative or inflammatory components as described in the invention.

[17] In particular, we have found that compounds in which R₂ is R₂-2 are particularly potent inhibitors of both ROCK1 and ROCK2, and that these agents inhibit the migration of neutrophils toward multiple chemotactic stimuli and inhibit the secretion of the cytokines IL-1β, TNF-α and IL-9 from LPS-stimulated human monocytes. Compounds in which Ar is heteroaryl, particularly 6,5-fused bicyclic heteroaryl, are especially preferred. These compounds are of particular value in addressing conditions with an inflammatory component.

Compounds exemplifying embodiment 17 include compounds 2.020, 2.021, 2.022, 2.026, 2.031, 2.033, 2.034, 2.038, 2.039, 2.040, 2.041, 2.043, 2.044, 2.054, 2.058, 2.059, 2.060, 2.063, 2.064, 2.066, 2.067, 2.068, 2.069, 2.070, 2.071, 2.072, 2.073, 2.076, 2.077, 2.078, 2.079, 2.080, 2.081, 2.082, 2.087, 2.092, 2.093, 2.094, 2.095, 2.096, 2.097, 2.098, 2.099, and 2.100.

[18] In another embodiment, we have found that compounds of Formula IIc are potent and selective inhibitors of ROCK2, with comparatively lower inhibitory potency against ROCK1. We have demonstrated that compounds of this class typically show good smooth muscle relaxation properties and that smooth muscle relaxation effects in this class are generally correlated with ROCK2 potency. Compounds in which Ar is phenyl are particularly preferred, and compounds bearing one polar group X1 in the 3-position and a second group X2 in the 4-position are most preferred. Compounds of this embodiment are of particular value in addressing conditions where relaxation of smooth muscle, in particular vascular and bronchial smooth muscle, is of highest importance.

Compounds exemplifying embodiment 18 include compounds 1.075, 1.077, 1.090, 1.091, 1.094, 1.095, 1.107, 1.109, 1.117, 1.118, 1.124, 1.152, 1.153, 1.157, 1.158, 1.165, 1.168, 1.176, 1.181, 1.182, 1.184, 1.185, 1.186, 1.187, 1.195, 1.196, 1.197, 1.198, 1.199, 1.200, 1.201, 1.213, 1.214, 1.215, 1.217, 1.218, 1.219, 1.223, 1.224, 1.228, 1.229, 1.230, 1.233, 1.234, 1.236, 1.237, 1.238, 1.239, 1.240, 1.253, 1.255, 1.261, 1.269, 1.270, 1.272, 1.274, 1.275, 1.280, and 1.282.

[19] In another embodiment, the inventors have found that compounds of Formula IIb are potent mixed inhibitors of ROCK1 and ROCK2, display additional inhibitory activity against the kinases Akt3 and p70S6K, and that these compounds generally display potent antiproliferative activity in models of smooth muscle cell proliferation. Compounds of this class are of particular value in addressing conditions in which an antiproliferative component is desired in combination with a smooth muscle relaxing effect.

Compounds exemplifying embodiment 19 include compounds 1.074, 1.076, 1.092, 1.093, 1.096, 1.097, 1.106, 1.108, 1.113, 1.115, 1.116, 1.123, 1.125, 1.126, 1.127, 1.128, 1.129, 1.139, 1.140, 1.147, 1.159, 1.160, 1.161, 1.162, 1.174, 1.188, 1.189, 1.193, 1.194, 1.202, 1.205, 1.206, 1.207, 1.208, 1.211, 1.212, 1.221, 1.222, 1.225, 1.231, 1.232, 1.235, 1.244, 1.248, 1.249, 1.258, 1.259, 1.260, 1.262, 1.263, 1.264, 1.265, 1.266, 1.267, 1.268, 1.271, 1.273, 1.276, and 1.281.

[20] In another embodiment, the inventors have found that certain compounds of Formulae IIa, IIb, and IIc distribute preferentially to the lung on oral dosing. In particular, compounds in which Ar is a lipophilic bicyclic heteroaryl group are preferred for this dosing behavior. Compounds of this type are especially useful for treating diseases of the lung by oral dosing while minimizing impact on other tissues.

Compounds exemplifying embodiment 20 include compounds 1.107, 1.109, 1.165, 1.106, 1.108, 2.058, 1.162, 1.264, 1.268, 1.271, 1.273, 1.217, 1.269, 2.059, 2.060, 2.066, and 2.072.

As discussed above for the compounds of Formulae Ia, Ib, and Ic, preparation of compounds of Formulae IIa, IIb, and IIc can be problematic using methods commonly known in the art. The inventors have disclosed and exemplified in US2008/0214614A1 methods to allow successful protection, coupling, and deprotection sequence that allows the successful preparation of the compounds of Formulae IIb and IIc and the demonstration of their useful biological properties.

The present compounds are useful for both oral and topical use, including use by the inhalation route. To be therapeutically effective in in this way, the compounds must have both adequate potency and proper pharmacokinetic properties such as good permeability across the biological surface relevant to the delivery route. In general, compounds of Formulae I and II bearing polar functionality, particularly on Ar, have preferred absorption properties and are particularly suitable for topical use. In general, compounds bearing small lipophilic functional groups have good ROCK inhibitory potency.

R₁ substitution in Formula I and X in Formula II are important factors for pharmacokinetic properties and ROCK inhibitory potency. Specifically, compounds bearing polar functionality, especially those specified in the embodiments 11, 12, 13, 14, 15, and 16 in Formulae I and II, above, are particularly suitable for topical use with adequate ROCK inhibiting activity. Compounds bearing small lipophilic functional groups, as specified in the embodiments 11, 12, 13, 14, 15, and 16 in Formulae I and II, above, display ROCK inhibition with adequate permeability across biological surfaces. Compounds bearing substituents of both types are particularly preferred, and when R₁ (Formula I) or Ar (Formula II) is a phenyl ring, compounds with small lipophilic groups in the 4-position and polar functionality in the 3-position are most preferred.

Specific compounds illustrative of Formula I and Formula II are shown in the following Table A. The example compounds have been numbered in such a way that numbers of the form 1.nnn indicate compounds in which R₂ is R₂-1, numbers of the form 2.nnn indicate compounds in which R₂ is R₂-2, and so on in a similar fashion for the remaining compound numbers and groups R₂. In the following structures, hydrogens are omitted from the drawings for the sake of simplicity. Tautomers drawn represent all tautomers possible. Structures are drawn to indicate the preferred, stereochemistry; where stereoisomers may be generated in these compounds, structures are taken to mean any of the possible stereoisomers alone or a mixture of stereoisomers in any ratio.

TABLE A
Exemplified Compounds.

| | | Select |
|---|---|---|
| Compound | Structure | Embodiments 1-16 |
| 1.001 | 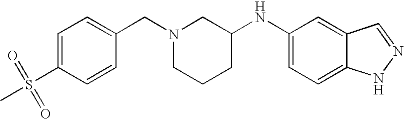 N-(1-(4-(methylsulfonyl)benzyl)piperidin-3-yl)-1H- indazol-5-amine | 1c, 7, 8, 9, 10, 12, 15c |
| 1.002 | 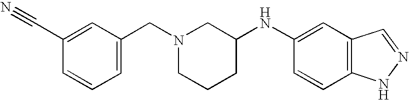 3-((3-(1H-indazol-5-ylamino)piperidin-1- yl)methyl)benzonitrile | 1c, 7, 8, 9, 10, 12, 15c |
| 1.003 | 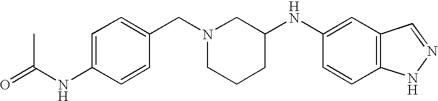 N-(4-((3-(1H-indazol-5-ylamino)piperidin-1- yl)methyl)phenyl)acetamide | 1c, 7, 8, 9, 10, 12a, 15c, 15d |
| 1.004 | 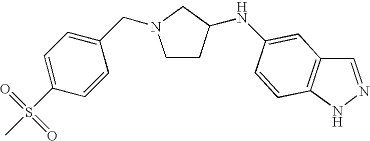 N-(1-(4-(methylsulfonyl)benzyl)pyrrolidin-3-yl)-1H- indazol-5-amine | 1c, 7, 8, 9, 10, 12, 15c |
| 1.005 | 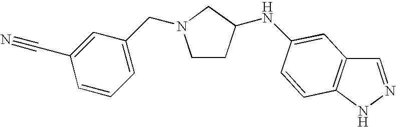 3-((3-(1H-indazol-5-ylamino)pyrrolidin-1- yl)methyl)benzonitrile | 1c, 7, 8, 9, 10, 12, 15c |
| 1.006 | 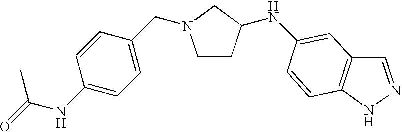 N-(4-((3-(1H-indazol-5-ylamino)pyrrolidin-1- yl)methyl)phenyl)acetamide | 1c, 7, 8, 9, 10, 12a, 15c, 15d |
| 1.007 | 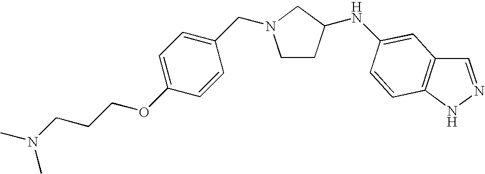 N-(1-(4-(3-(dimethylamino)propoxy)benzyl)pyrrolidin- 3-yl)-1H-indazol-5-amine | 1c, 7, 8, 9, 10, 12a, 15c, 15d |
| 1.008 | 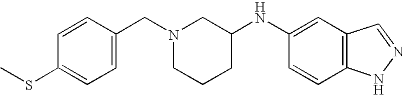 N-(1-(4-(methylthio)benzyl)piperidin-3-yl)-1H-indazol- 5-amine | 1c, 7, 8, 9, 10, 12b, 15c, 15e |
| 1.009 | 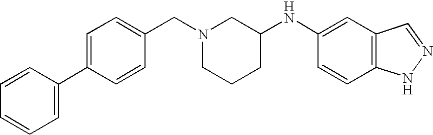 N-(1-(biphenyl-4-ylmethyl)piperidin-3-yl)-1H-indazol-5- amine | 1c, 7, 8, 9, 10, 11, 14c |
| 1.010 | 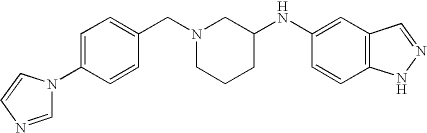 N-(1-(1H-imidazol-1-yl)benzyl)piperidin-3-yl)-1H- indazol-5-amine | 1c, 7, 8, 9, 10, 11, 14c |
| 1.011 | 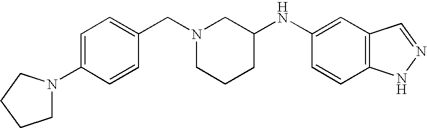 N-(1-(4-(pyrrolidin-1-yl)benzyl)piperidin-3-yl)-1H- indazol-5-amine | 1c, 7, 8, 9, 10, 11, 14c |
| 1.012 | 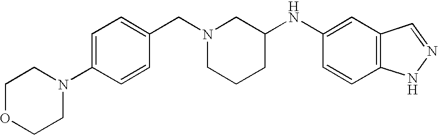 N-(1-(4-morpholinobenzyl)piperidin-3-yl)-1H-indazol-5- amine | 1c, 7, 8, 9, 10, 11, 14c |
| 1.013 | 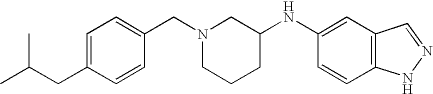 N-(1-(4-isobutylbenzyl)piperidin-3-yl)-1H-indazol-5- amine | 1c, 7, 8, 9, 10 |
| 1.014 | 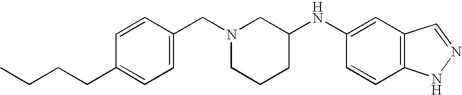 N-(1-(4-butylbenzyl)piperidin-3-yl)-1H-indazol-5-amine | 1c, 7, 8, 9, 10 |
| 1.015 | 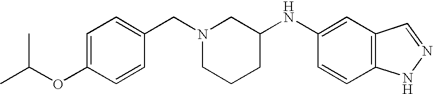 N-(1-(4-isopropoxybenzyl)piperidin-3-yl)-1H-indazol-5- amine | 1c, 7, 8, 9, 10 |
| 1.016 | 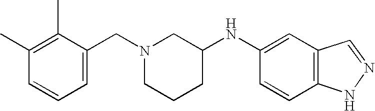 N-(1-(2,3-dimethylbenzyl)piperidin-3-yl)-1H-indazol-5- amine | 1c, 7, 8, 9, 10 |
| 1.017 | 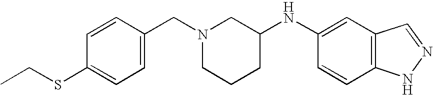 N-(1-(4-(ethylthio)benzyl)piperidin-3-yl)-1H-indazol-5- amine | 1c, 7, 8, 9, 10, 12b, 15c, 15e |
| 1.018 | 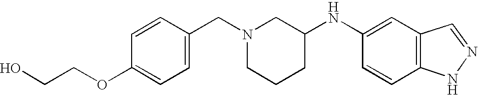 2-(4-((3-(1H-indazol-5-ylamino)piperidin-1-yl)methyl) phenoxy)ethanol | 1c, 7, 8, 9, 10, 12a, 15c, 15d |
| 1.019 | 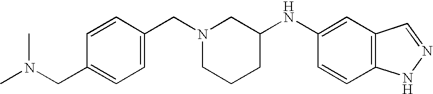 N-(1-(4-((dimethylamino)methyl)benzyl)piperidin-3-yl)- 1H-indazol-5-amine | 1c, 7, 8, 9, 10, 13, 16c |
| 1.020 | 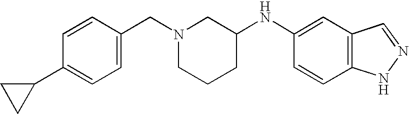 N-(1-(4-cyclopropylbenzyl)piperidin-3-yl)-1H-indazol-5- amine | 1c, 7, 8, 9, 10, 11, 14c |
| 1.021 | 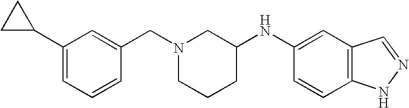 N-(1-(3-cyclopropylbenzyl)piperidin-3-yl)-1H-indazol-5- amine | 1c, 7, 8, 9, 10, 11, 14c |
| 1.022 | 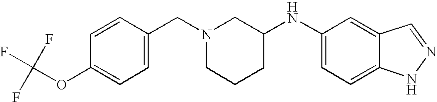 N-(1-(4-(trifluoromethoxy)benzyl)piperidin-3-yl)-1H- indazol-5-amine | 1c, 7, 8, 9, 10 |
| 1.023 | 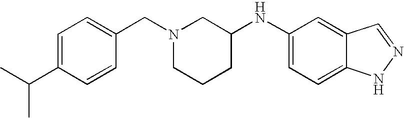 N-(1-(4-isopropylbenzyl)piperidin-3-yl)-1H-indazol-5- amine | 1c, 7, 8, 9, 10 |
| 1.024 | 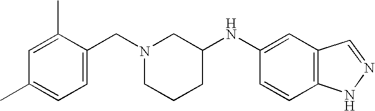 N-(1-(2,4-dimethylbenzyl)piperidin-3-yl)-1H-indazol-5- amine | 1c, 7, 8, 9, 10 |
| 1.025 | 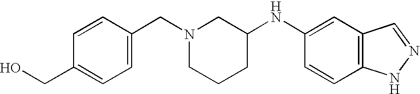 (4-((3-(1H-indazol-5-ylamino)piperidin-1- yl)methyl)phenyl)methanol | 1c, 7, 8, 9, 10 |
| 1.026 | 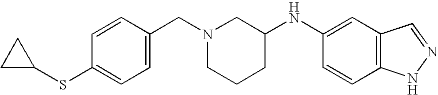 N-(1-(4-(cyclopropylthio)benzyl)piperidin-3-yl)-1H- indazol-5-amine | 1c, 7, 8, 9, 10, 12b, 15c, 15e |
| 1.027 | 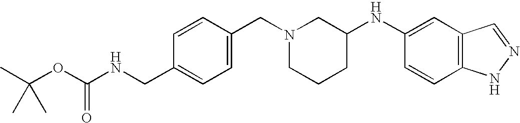 tert-butyl 4-((3-(1H-indazol-5-ylamino)piperidin-1- yl)methyl)benzylcarbamate | 1c, 7, 8, 9, 10, 13, 16c |
| 1.028 | 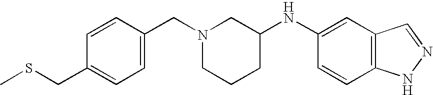 N-(1-(4-(methylthiomethyl)benzyl)piperidin-3-yl)-1H- indazol-5-amine | 1c, 7, 8, 9, 10, 13, 16c |
| 1.029 | 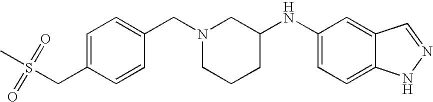 N-(1-(4-(methylsulfonylmethyl)benzyl)piperidin-3-yl)- 1H-indazol-5-amine | 1c, 7, 8, 9, 10, 13, 16c |
| 1.030 | 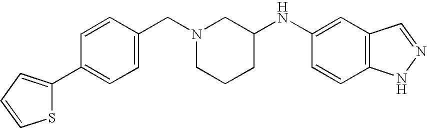 N-(1-(4-(thiophen-2-yl)benzyl)piperidin-3-yl)-1H- indazol-5-amine | 1c, 7, 8, 9, 10, 11, 14c |
| 1.031 | 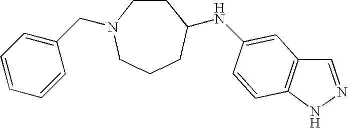 N-(1-benzylazepan-4-yl)-1H-indazol-5-amine | 1c, 7, 8, 9, 10 |
| 1.032 | 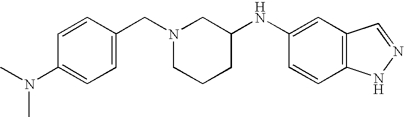 N-(1-(4-(dimethylamino)benzyl)piperidin-3-yl)-1H- indazol-5-amine | 1c, 7, 8, 9, 10 |
| 1.033 | 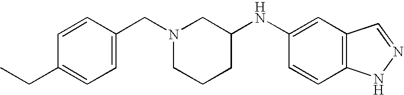 N-(1-(4-ethylbenzyl)piperidin-3-yl)-1H-indazol-5-amine | 1c, 7, 8, 9, 10 |
| 1.034 | 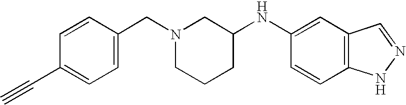 N-(1-(4-ethynylbenzyl)piperidin-3-yl)-1H-indazol-5- amine | 1c, 7, 8, 9, 10, 11, 14c |
| 1.035 | 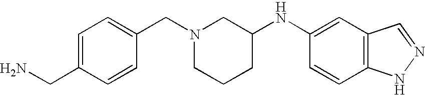 N-(1-(4-(aminomethyl)benzyl)piperidin-3-yl)-1H- indazol-5-amine | 1c, 7, 8, 9, 10, 13, 16c |
| 1.036 | 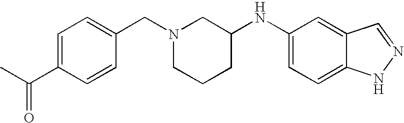 1-(4-((3-(1H-indazol-5-ylamino)piperidin-1- yl)methyl)phenyl)ethanone | 1c, 7, 8, 9, 10 |
| 1.037 | 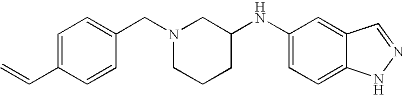 N-(1-(4-vinylbenzyl)piperidin-3-yl)-1H-indazol-5-amine | 1c, 7, 8, 9, 10, 11, 14c |
| 1.038 | 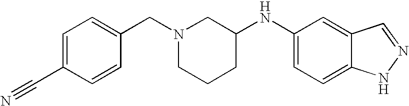 4-((3-(1H-indazol-5-ylamino)piperidin-1- yl)methyl)benzonitrile | 1c, 7, 8, 9, 10, 12, 15c |
| 1.039 | 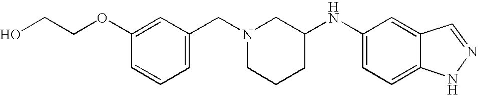 2-(3-((3-(1H-indazol-5-ylamino)piperidin-1-yl)methyl) phenoxy)ethanol | 1c, 7, 8, 9, 10, 12a, 15c, 15d |
| 1.040 | 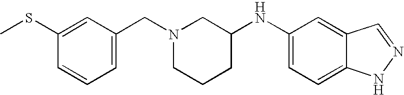 N-(1-(3-(methylthio)benzyl)piperidin-3-yl)-1H-indazol- 5-amine | 1c, 7, 8, 9, 10, 12b, 15c, 15e |
| 1.041 | 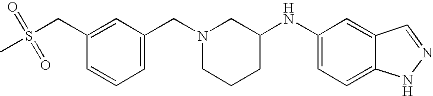 N-(1-(3-(methylsulfonylmethyl)benzyl)piperidin-3-yl)- 1H-indazol-5-amine | 1c, 7, 8, 9, 10, 13, 16c |
| 1.042 | 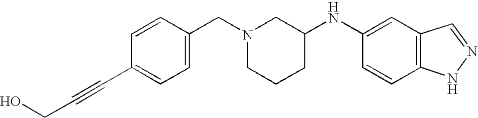 3-(4-((3-(1H-indazol-5-ylamino)piperidin-1- yl)methyl)phenyl)prop-2-yn-1-ol | 1c, 7, 8, 9, 10, 13, 16c |
| 1.043 | 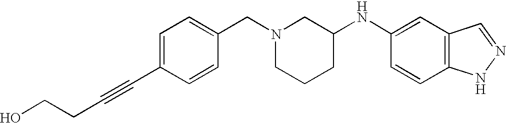 4-(4-((3-(1H-indazol-5-ylamino)piperidin-1- yl)methyl)phenyl)but-3-yn-1-ol | 1c, 7, 8, 9, 10, 13, 16c |
| 1.044 | 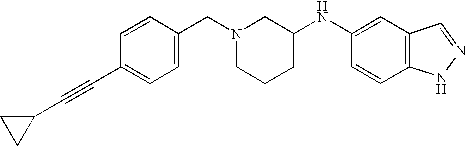 N-(1-(4-(cyclopropylethynyl)benzyl)piperidin-3-yl)-1H- indazol-5-amine | 1c, 7, 8, 9, 10, 11, 14c |
| 1.045 | 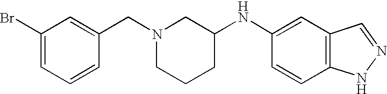 N-(1-(3-bromobenzyl)piperidin-3-yl)-1H-indazol-5- amine | 1c, 7, 8, 9, 10 |
| 1.046 | 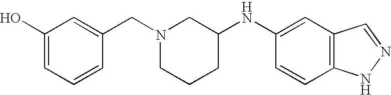 3-((3-(1H-indazol-5-ylamino)piperidin-1- yl)methyl)phenol | 1c, 7, 8, 9, 10 |
| 1.047 | 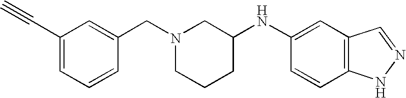 N-(1-(3-ethynylbenzyl)piperidin-3-yl)-1H-indazol-5- amine | 1c, 7, 8, 9, 10, 11, 14c |
| 1.048 | 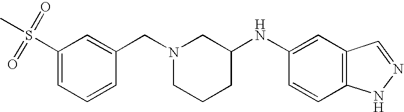 N-(1-(3-(methylsulfonyl)benzyl)piperidin-3-yl)-1H- indazol-5-amine | 1c, 7, 8, 9, 10, 12, 15c |
| 1.049 | 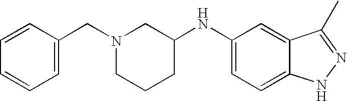 N-(1-benzylpiperidin-3-yl)-3-methyl-1H-indazol-5- amine | 1a, 6a, 8, 9, 10 |
| 1.050 | 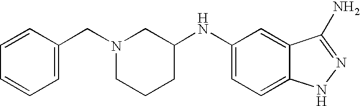 N5-(1-benzylpiperidin-3-yl)-1H-indazole-3,5-diamine | 1b, 6b, 8, 9, 10 |
| 1.051 | 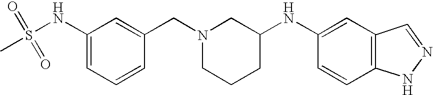 N-(3-((3-(1H-indazol-5-ylamino)piperidin-1- yl)methyl)phenyl)methanesulfonamide | 1c, 7, 8, 9, 10, 12a, 15c, 15d |
| 1.052 | 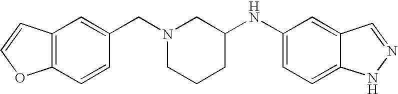 N-(1-(benzofuran-5-ylmethyl)piperidin-3-yl)-1H- indazol-5-amine | 1c, 7, 8, 9, 10 |
| 1.053 | 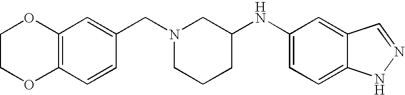 N-(1-((2,3-dihydrobenzo[b][1,4]dioxin-6- yl)methyl)piperidin-3-yl)-1H-indazol-5-amine | 1c, 7, 8, 9, 10 |
| 1.054 | 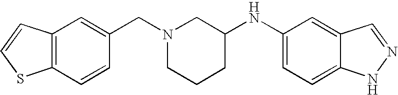 N-(1-(benzo[b]thiophen-5-ylmethyl)piperidin-3-yl)-1H- indazol-5-amine | 1c, 7, 8, 9, 10 |
| 1.055 | 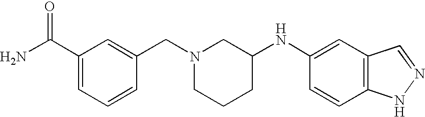 3-((3-(1H-indazol-5-ylamino)piperidin-1- yl)methyl)benzamide | 1c, 7, 8, 9, 10, 12, 15c |
| 1.056 | 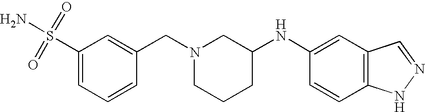 3-((3-(1H-indazol-5-ylamino)piperidin-1- yl)methyl)benzenesulfonamide | 1c, 7, 8, 9, 10, 12, 15c |
| 1.057 | 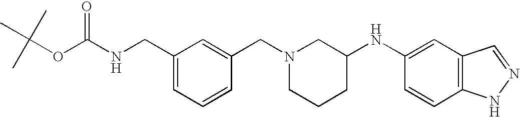 tert-butyl 3-((3-(1H-indazol-5-ylamino)piperidin-1- yl)methyl)benzylcarbamate | 1c, 7, 8, 9, 10, 13, 16c |
| 1.058 | 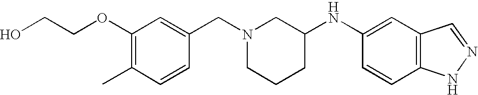 2-(5-((3-(1H-indazol-5-ylamino)piperidin-1-yl)methyl)- 2-methylphenoxy)ethanol | 1c, 7, 8, 9, 10, 12a, 15c, 15d |
| 1.059 | 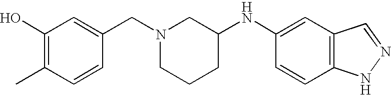 5-((3-(1H-indazol-5-ylamino)piperidin-1-yl)methyl)-2- methylphenol | 1c, 7, 8, 9, 10 |
| 1.060 | 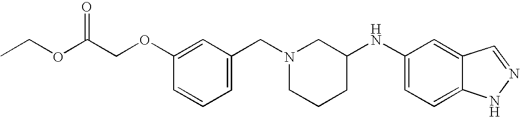 ethyl 2-(3-((3-(1H-indazol-5-ylamino)piperidin-1- yl)methyl)phenoxy)acetate | 1c, 7, 8, 9, 10, 12a, 15c, 15d |
| 1.061 | 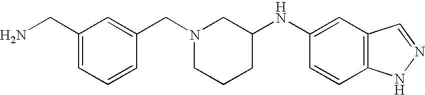 N-(1-(3-(aminomethyl)benzyl)piperidin-3-yl)-1H- indazol-5-amine | 1c, 7, 8, 9, 10, 13, 16c |
| 1.062 | 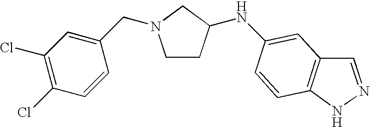 N-(1-(3,4-dichlorobenzyl)pyrrolidin-3-yl)-1H-indazol-5- amine | 1c, 7, 8, 9, 10 |
| 1.063 | 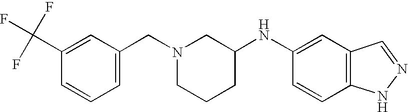 N-(1-(3-(trifluoromethyl)benzyl)piperidin-3-yl)-1H- indazol-5-amine | 1c, 7, 8, 9, 10 |
| 1.064 | 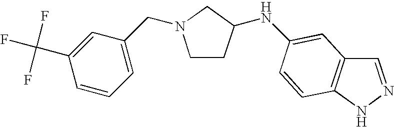 N-(1-(3-(trifluoromethyl)benzyl)pyrrolidin-3-yl)-1H- indazol-5-amine | 1c, 7, 8, 9, 10 |
| 1.065 | 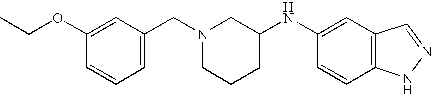 N-(1-(3-ethoxybenzyl)piperidin-3-yl)-1H-indazol-5- amine | 1c, 7, 8, 9, 10 |
| 1.066 | 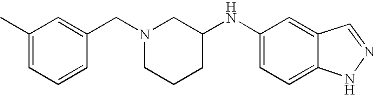 N-(1-(3-methylbenzyl)piperidin-3-yl)-1H-indazol-5- amine | 1c, 7, 8, 9, 10 |
| 1.067 | 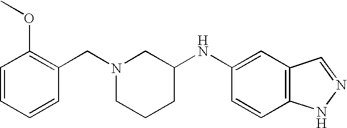 N-(1-(2-methoxybenzyl)piperidin-3-yl)-1H-indazol-5- amine | 1c, 7, 8, 9, 10 |
| 1.068 | 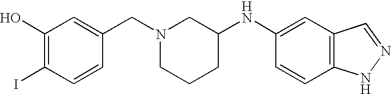 5-((3-(1H-indazol-5-ylamino)piperidin-1-yl)methyl)-2- iodophenol | 1c, 7, 8, 9, 10 |
| 1.069 | 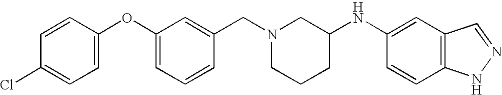 N-(1-(3-(4-chlorophenoxy)benzyl)piperidin-3-yl)-1H- indazol-5-amine | 1c, 7, 8, 9, 10 |
| 1.070 | 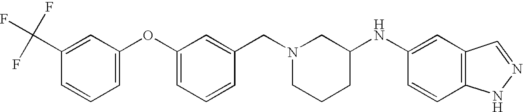 N-(1-(3-(3-(trifluoromethyi)phenoxy)benzyl)piperidin-3- yl)-1H-indazol-5-amine | 1c, 7, 8, 9, 10 |
| 1.071 | 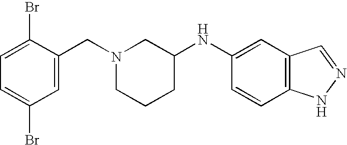 N-(1-(2,5-dibromobenzyl)piperidin-3-yl)-1H-indazol-5- amine | 1c, 7, 8, 9, 10 |
| 1.072 | 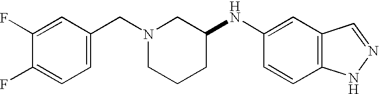 (S)-N-(1-(3,4-difluorobenzyl)piperidin-3-yl)-1H-indazol- 5-amine | 1c, 7, 8, 9, 10 |
| 1.073 | 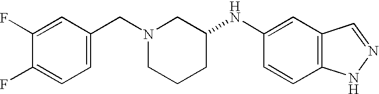 (R)-N-(1-(3,4-difluorobenzyl)piperidin-3-yl)-1H-indazol- 5-amine | 1c, 7, 8, 9, 10 |
| 1.074 | 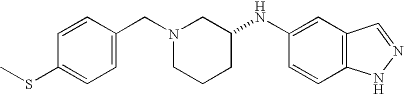 (R)-N-(1-(4-(methylthio)benzyl)piperidin-3-yl)-1H- indazol-5-amine | 1c, 7, 8, 9, 10, 12b, 15c, 15e |
| 1.075 | 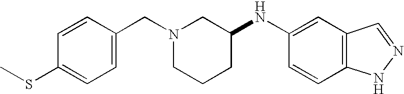 (S)-N-(1-(4-(methylthio)benzyl)piperidin-3-yl)-1H- indazol-5-amine | 1c, 7, 8, 9, 10, 12b, 15c, 15e |
| 1.076 | 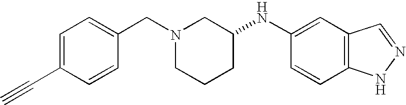 (R)-N-(1-(4-ethynylbenzyl)piperidin-3-yl)-1H-indazol-5- amine | 1c, 7, 8, 9, 10, 11, 14c |
| 1.077 | 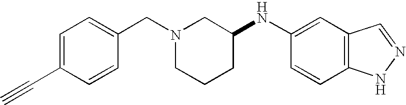 (S)-N-(1-(4-ethynylbenzyl)piperidin-3-yl)-1H-indazol-5- amine | 1c, 7, 8, 9, 10, 11, 14c |
| 1.078 | 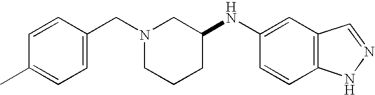 (S)-N-(1-(4-methylbenzyl)piperidin-3-yl)-1H-indazol-5- amine | 1c, 7, 8, 9, 10 |
| 1.079 | 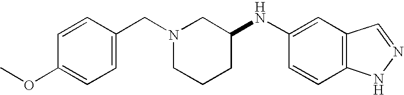 (S)-N-(1-(4-methoxybenzyl)piperidin-3-yl)-1H-indazol- 5-amine | 1c, 7, 8, 9, 10 |
| 1.080 | 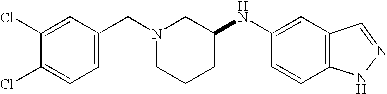 (S)-N-(1-(3,4-dichlorobenzyl)piperidin-3-yl)-1H- indazol-5-amine | 1c, 7, 8, 9, 10 |
| 1.082 | 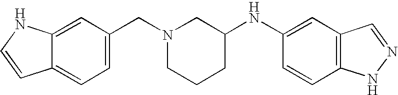 N-(1-((1H-indol-6-yl)methyl)piperidin-3-yl)-1H-indazol- 5-amine | 1c, 7, 8, 9, 10 |
| 1.083 | 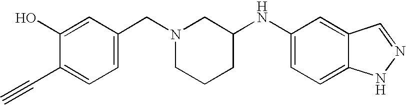 5-((3-(1H-indazol-5-ylamino)piperidin-1-yl)methyl)-2- ethynylphenol | 1c, 7, 8, 9, 10, 11, 14c |
| 1.084 | 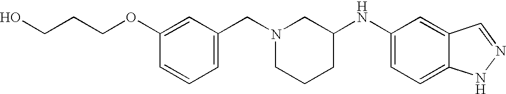 3-(3-((3-(1H-indazol-5-ylamino)piperidin-1-yl)methyl) phenoxy)propan-1-ol | 1c, 7, 8, 9, 10, 12a, 15c, 15d |
| 1.085 | 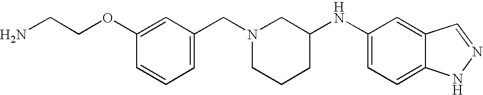 N-(1-(3-(2-aminoethoxy)benzyl)piperidin-3-yl)-1H- indazol-5-amine | 1c, 7, 8, 9, 10, 12a, 15c, 15d |
| 1.086 | 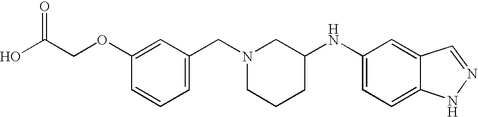 2-(3-((3-(1H-indazol-5-ylamino)piperidin-1-yl)methyl) phenoxy)acetic acid | 1c, 7, 8, 9, 10, 12a, 15c, 15d |
| 1.087 | 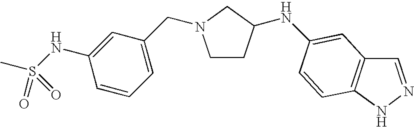 N-(3-((3-(1H-indazol-5-ylamino)pyrrolidin-1- yl)methyl)phenyl)methanesulfonamide | 1c, 7, 8, 9, 10, 12a, 15c, 15d |
| 1.088 | 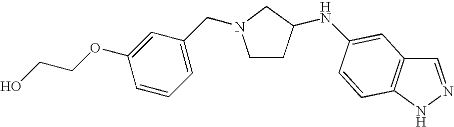 2-(3-((3-(1H-indazol-5-ylamino)pyrrolidin-1-yl)methyl) phenoxy)ethanol | 1c, 7, 8, 9, 10, 12a, 15c, 15d |
| 1.089 | 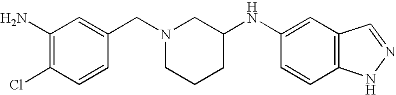 N-(1-(3-amino-4-chlorobenzyl)piperidin-3-yl)-1H- indazol-5-amine | 1c, 7, 8, 9, 10 |
| 1.090 | 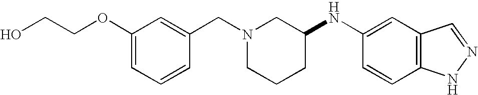 (S)-2-(3-((3-(1H-indazol-5-ylamino)piperidin-1- yl)methyl)phenoxy)ethanol | 1c, 7, 8, 9, 10, 12a, 15c, 15d |
| 1.091 | 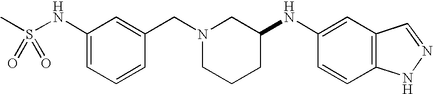 (S)-N-(3-((3-(1H-indazol-5-ylamino)piperidin-1- yl)methyl)phenyl)methanesulfonamide | 1c, 7, 8, 9, 10, 12a, 15c, 15d |
| 1.092 | 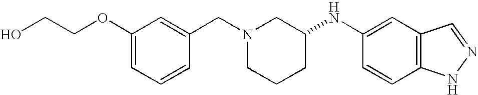 (R)-2-(3-((3-(1H-indazol-5-ylamino)piperidin-1- yl)methyl)phenoxy)ethanol | 1c, 7, 8, 9, 10, 12a, 15c, 15d |
| 1.093 | 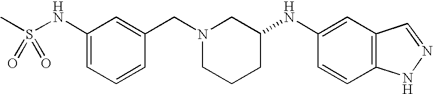 (R)-N-(3-((3-(1H-indazol-5-ylamino)piperidin-1- yl)methyl)phenyl)methanesulfonamide | 1c, 7, 8, 9, 10, 12a, 15c, 15d |
| 1.094 | 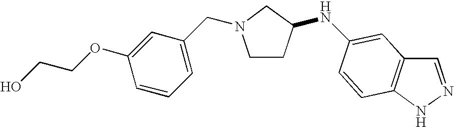 (S)-2-(3-((3-(1H-indazol-5-ylamino)pyrrolidin-1- yl)methyl)phenoxy)ethanol | 1c, 7, 8, 9, 10, 12a, 15c, 15d |
| 1.095 | 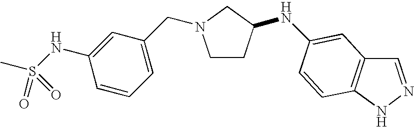 (S)-N-(3-((3-(1H-indazol-5-ylamino)pyrrolidin-1- yl)methyl)phenyl)methanesulfonamide | 1c, 7, 8, 9, 10, 12a, 15c, 15d |
| 1.096 | 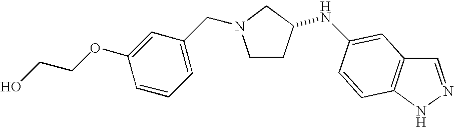 (R)-2-(3-((3-(1H-indazol-5-ylamino)pyrrolidin-1- yl)methyl)phenoxy)ethanol | 1c, 7, 8, 9, 10, 12a, 15c, 15d |
| 1.097 | 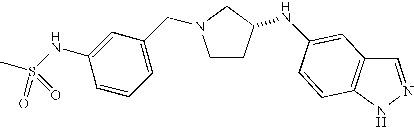 (R)-N-(3-((3-(1H-indazol-5-ylamino)pyrrolidin-1- yl)methyl)phenyl)methanesulfonamide | 1c, 7, 8, 9, 10, 12a, 15c, 15d |
| 1.098 | 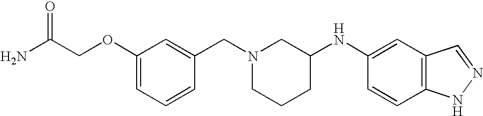 2-(3-((3-(1H-indazol-5-ylamino)piperidin-1- yl)methyl)phenoxy)acetamide | 1c, 7, 8, 9, 10, 12a, 15c, 15d |
| 1.099 | 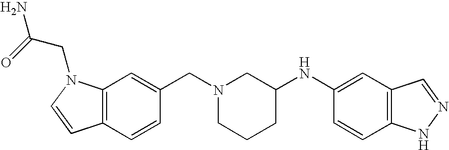 2-(6-((3-(1H-indazol-5-ylamino)piperidin-1-yl)methyl)- 1H-indol-1-yl)acetamide | 1c, 7, 8, 9, 10, 13, 16c |
| 1.100 | 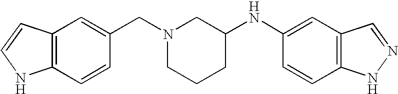 N-(1-((1H-indol-5-yl)methyl)piperidin-3-yl)-1H-indazol- 5-amine | 1c, 7, 8, 9, 10, 13, 16c |
| 1.101 | 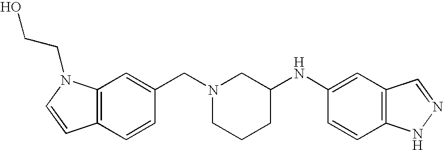 2-(6-((3-(1H-indazol-5-ylamino)piperidin-1-yl)methyl)- 1H-indol-1-yl)ethanol | 1c, 7, 8, 9, 10, 13, 16c |
| 1.102 | 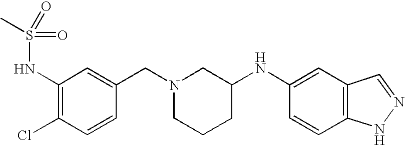 N-(5-((3-(1H-indazol-5-ylamino)piperidin-1-yl)methyl)- 2-chlorophenyl)methanesulfonamide | 1c, 7, 8, 9, 10, 12a, 15c, 15d |
| 1.103 | 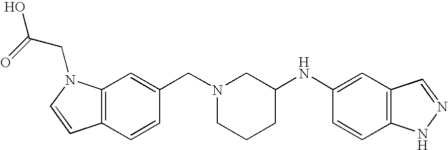 2-(6-((3-(1H-indazol-5-ylamino)piperidin-1-yl)methyl)- 1H-indol-1-yl)acetic acid | 1c, 7, 8, 9, 10, 13, 16c |
| 1.104 | 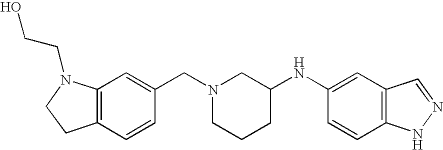 2-(6-((3-(1H-indazol-5-ylamino)piperidin-1- yl)methyl)indolin-1-yl)ethanol | 1c, 7, 8, 9, 10, 13, 16c |
| 1.105 | 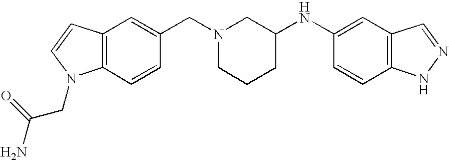 2-(5-((3-(1H-indazol-5-ylamino)piperidin-1-yl)methyl)- 1H-indol-1-yl)acetamide | 1c, 7, 8, 9, 10, 13, 16c |
| 1.106 | 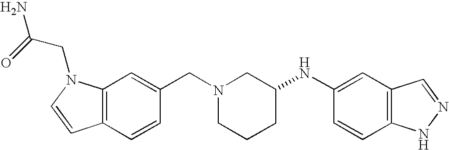 (R)-2-(6-((3-(1H-indazol-5-ylamino)piperidin-1- yl)methyl)-1H-indol-1-yl)acetamide | 1c, 7, 8, 9, 10, 13, 16c |
| 1.107 | 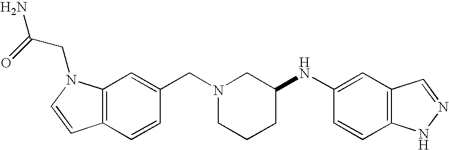 (S)-2-(6-((3-(1H-indazol-5-ylamino)piperidin-1- yl)methyl)-1H-indol-1-yl)acetamide | 1c, 7, 8, 9, 10, 13, 16c |
| 1.108 | 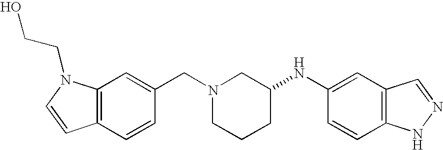 (R)-2-(6-((3-(1H-indazol-5-ylamino)piperidin-1- yl)methyl)-1H-indol-1-yl)ethanol | 1c, 7, 8, 9, 10, 13, 16c |
| 1.109 | 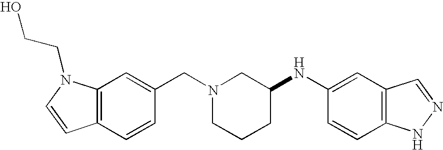 (S)-2-(6-((3-(1H-indazol-5-ylamino)piperidin-1- yl)methyl)-1H-indol-1-yl)ethanol | 1c, 7, 8, 9, 10, 13, 16c |
| 1.110 | 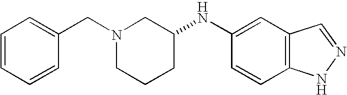 (R)-N-(1-benzylpiperidin-3-yl)-1H-indazol-5-amine | 1c, 7, 8, 9, 10 |
| 1.111 | 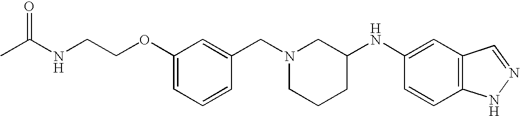 N-(2-(3-((3-(1H-indazol-5-ylamino)piperidin-1- yl)methyl)phenoxy)ethyl)acetamide | 1c, 7, 8, 9, 10, 12a, 15c, 15d |
| 1.112 | 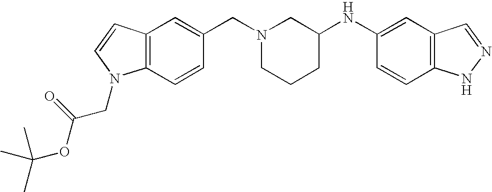 tert-butyl 2-(5-((3-(1H-indazol-5-ylamino)piperidin-1- yl)methyl)-1H-indol-1-yl)acetate | 1c, 7, 8, 9, 10, 13, 16c |
| 1.113 | 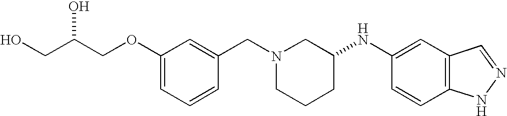 (S)-3-(3-(((R)-3-(1H-indazol-5-ylamino)piperidin-1- yl)methyl)phenoxy)propane-1,2-diol | 1c, 7, 8, 9, 10, 12a, 15c, 15d |
| 1.114 | 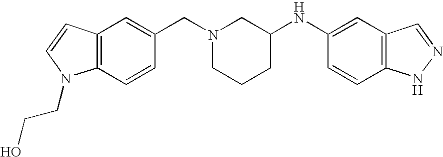 2-(5-((3-(1H-indazol-5-ylamino)piperidin-1-yl)methyl)- 1H-indol-1-yl)ethanol | 1c, 7, 8, 9, 10, 13, 16c |
| 1.115 | 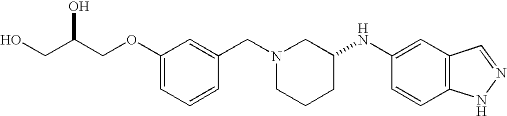 (R)-3-(3-(((R)-3-(1H-indazol-5-ylamino)piperidin-1- yl)methyl)phenoxy)propane-1,2-diol | 1c, 7, 8, 9, 10, 12a, 15c, 15d |
| 1.116 | 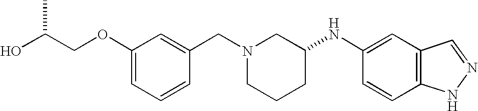 (R)-1-(3-(((R)-3-(1H-indazol-5-ylamino)piperidin-1- yl)methyl)phenoxy)propan-2-ol | 1c, 7, 8, 9, 10, 12a, 15c, 15d |
| 1.117 | 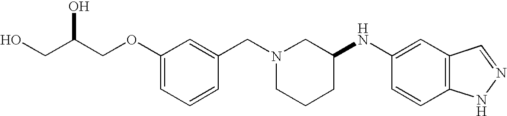 (R)-3-(3-(((S)-3-(1H-indazol-5-ylamino)piperidin-1- yl)methyl)phenoxy)propane-1,2-diol | 1c, 7, 8, 9, 10, 12a, 15c, 15d |
| 1.118 | 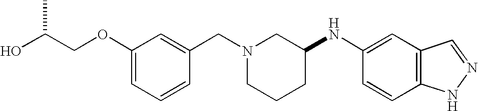 (R)-1-(3-(((S)-3-(1H-indazol-5-ylamino)piperidin-1- yl)methyl)phenoxy)propan-2-ol | 1c, 7, 8, 9, 10, 12a, 15c, 15d |
| 1.119 | 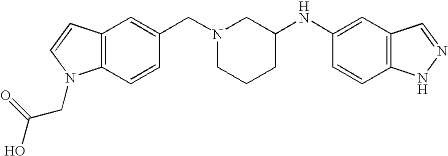 2-(5-((3-(1H-indazol-5-ylamino)piperidin-1-yl)methyl)- 1H-indol-1-yl)acetic acid | 1c, 7, 8, 9, 10, 13, 16c |
| 1.120 | 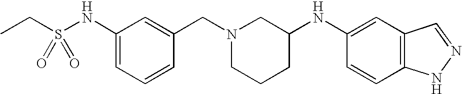 N-(3-((3-(1H-indazol-5-ylamino)piperidin-1- yl)methyl)phenyl)ethanesulfonamide | 1c, 7, 8, 9, 10, 12a, 15c, 15d |
| 1.121 | 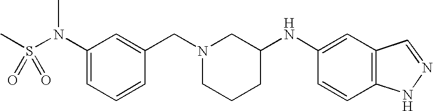 N-(3-((3-(1H-indazol-5-ylamino)piperidin-1- yl)methyl)phenyl)-N-methylmethanesulfonamide | 1c, 7, 8, 9, 10, 12a, 15c, 15d |
| 1.122 | 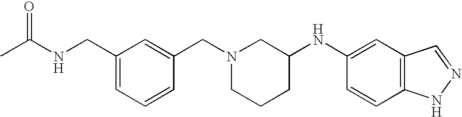 N-(3-((3-(1H-indazol-5-ylamino)piperidin-1- yl)methyl)benzyl)acetamide | 1c, 7, 8, 9, 10, 13, 16c |
| 1.123 | 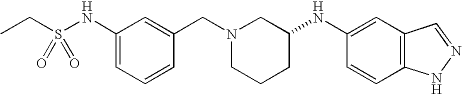 (R)-N-(3-((3-(1H-indazol-5-ylamino)piperidin-1- yl)methyl)phenyl)ethanesulfonamide | 1c, 7, 8, 9, 10, 12a, 15c, 15d |
| 1.124 | 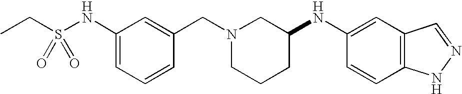 (S)-N-(3-((3-(1H-indazol-5-ylamino)piperidin-1- yl)methyl)phenyl)ethanesulfonamide | 1c, 7, 8, 9, 10, 12a, 15c, 15d |
| 1.125 | 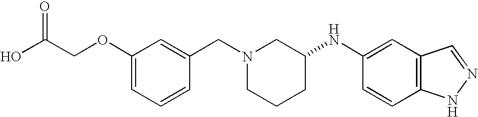 (R)-2-(3-((3-(1H-indazol-5-ylamino)piperidin-1- yl)methyl)phenoxy)acetic acid | 1c, 7, 8, 9, 10, 12a, 15c, 15d |
| 1.126 | 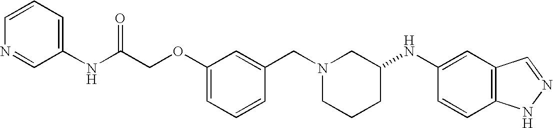 (R)-2-(3-((3-(1H-indazol-5-ylamino)piperidin-1- yl)methyl)phenoxy)-N-(pyridin-3-yl)acetamide | 1c, 7, 8, 9, 10, 12a, 15c, 15d |
| 1.127 | 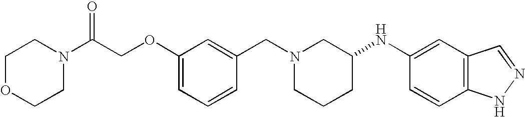 (R)-2-(3-((3-(1H-indazol-5-ylamino)piperidin-1- yl)methyl)phenoxy)-l-morpholinoethanone | 1c, 7, 8, 9, 10, 12a, 15c, 15d |
| 1.128 | 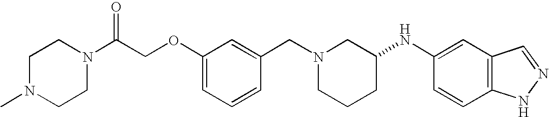 (R)-2-(3-((3-(1H-indazol-5-ylamino)piperidin-1- yl)methyl)phenoxy)-1-(4-methylpiperazin-1- yl)ethanone | 1c, 7, 8, 9, 10, 12a, 15c, 15d |
| 1.129 | 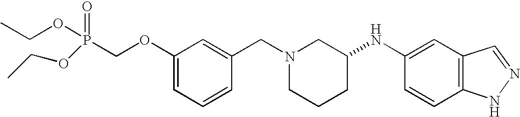 (R)-diethyl (3-((3-(1H-indazol-5-ylamino)piperidin-1- yl)methyl)phenoxy)methylphosphonate | 1c, 7, 8, 9, 10, 12a, 15c, 15d |
| 1.130 | 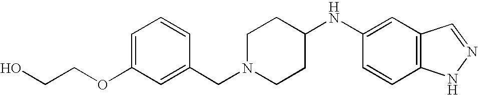 2-(3-((4-(1H-indazol-5-ylamino)piperidin-1- yl)methyl)phenoxy)ethanol | 1c, 7, 8, 9, 10, 12a, 15c, 15d |
| 1.131 | 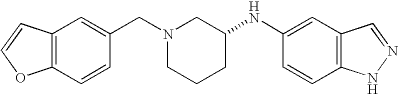 (R)-N-(1-(benzofuran-5-ylmethyl)piperidin-3-yl)-1H- indazol-5-amine | 1c, 7, 8, 9, 10 |
| 1.132 | 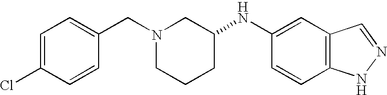 (R)-N-(1-(4-chlorobenzyl)piperidin-3-yl)-1H-indazol-5- amine | 1c, 7, 8, 9, 10 |
| 1.133 | 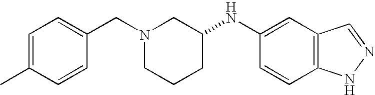 (R)-N-(1-(4-methylbenzyl)piperidin-3-yl)-1H-indazol-5- amine | 1c, 7, 8, 9, 10 |
| 1.134 | 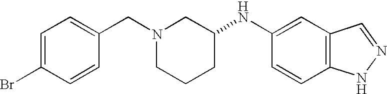 (R)-N-(1-(4-bromobenzyl)piperidin-3-yl)-1H-indazol-5- amine | 1c, 7, 8, 9, 10 |
| 1.136 | 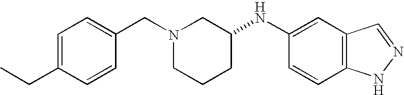 (R)-N-(1-(4-ethylbenzyl)piperidin-3-yl)-1H-indazol-5- amine | 1c, 7, 8, 9, 10 |
| 1.137 | 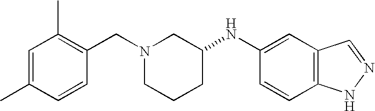 (R)-N-(1-(2,4-dimethylbenzyl)piperidin-3-yl)-1H- indazol-5-amine | 1c, 7, 8, 9, 10 |
| 1.138 | 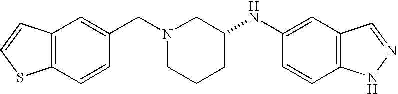 (R)-N-(1-(benzo[b]thiophen-5-ylmethyl)piperidin-3-yl)- 1H-indazol-5-amine | 1c, 7, 8, 9, 10 |
| 1.139 | 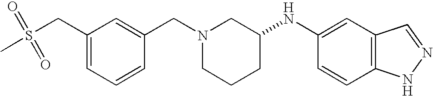 (R)-N-(1-(3-(methylsulfonylmethyl)benzyl)piperidin-3- yl)-1H-indazol-5-amine | 1c, 7, 8, 9, 10, 12, 15c |
| 1.140 | 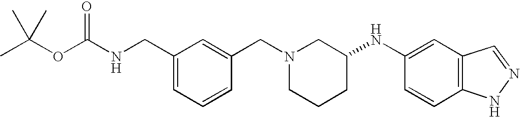 (R)-tert-butyl 3-((3-(1H-indazol-5-ylamino)piperidin-1- yl)methyl)benzylcarbamate | 1c, 7, 8, 9, 10, 13, 16c |
| 1.141 | 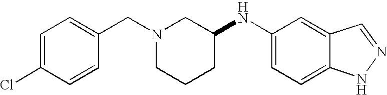 (S)-N-(1-(4-chlorobenzyl)piperidin-3-yl)-1H-indazol-5- amine | 1c, 7, 8, 9, 10 |
| 1.142 | 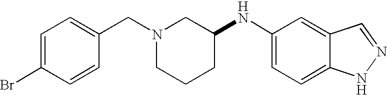 (S)-N-(1-(4-bromobenzyl)piperidin-3-yl)-1H-indazol-5- amine | 1c, 7, 8, 9, 10 |
| 1.143 | 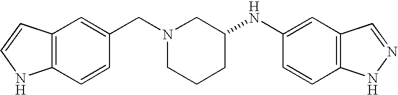 (R)-N-(1-((1H-indol-5-yl)methyl)piperidin-3-yl)-1H- indazol-5-amine | 1c, 7, 8, 9, 10, 13, 16c |
| 1.144 | 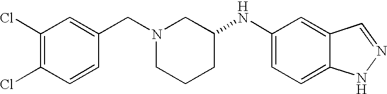 (R)-N-(1-(3,4-dichlorobenzyl)piperidin-3-yl)-1H- indazol-5-amine | 1c, 7, 8, 9, 10 |
| 1.145 | 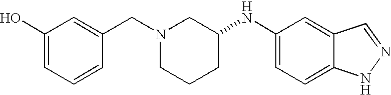 (R)-3-((3-(1H-indazol-5-ylamino)piperidin-1- yl)methyl)phenol | 1c, 7, 8, 9, 10 |
| 1.146 | 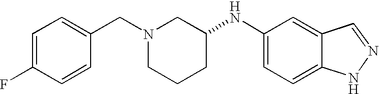 (R)-N-(1-(4-fluorobenzyl)piperidin-3-yl)-1H-indazol-5- amine | 1c, 7, 8, 9, 10 |
| 1.147 | 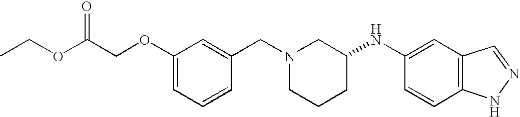 (R)-ethyl 2-(3-((3-(1H-indazol-5-ylamino)piperidin-1- yl)methyl)phenoxy)acetate | 1c, 7, 8, 9, 10, 12a, 15c, 15d |
| 1.148 | 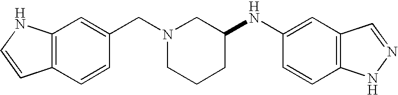 (S)-N-(1-((1H-indol-6-yl)methyl)piperidin-3-yl)-1H- indazol-5-amine | 1c, 7, 8, 9, 10 |
| 1.149 | 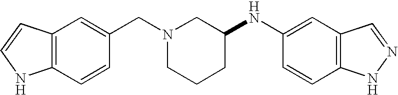 (S)-N-(1-((1H-indol-5-yl)methyl)piperidin-3-yl)-1H- indazol-5-amine | 1c, 7, 8, 9, 10 |
| 1.150 | 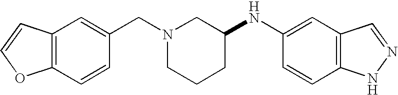 (S)-N-(1-(benzofuran-5-ylmethyl)piperidin-3-yl)-1H- indazol-5-amine | 1c, 7, 8, 9, 10 |
| 1.151 | 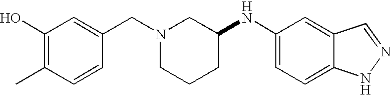 (S)-5-((3-(1H-indazol-5-ylamino)piperidin-1-yl)methyl)- 2-methylphenol | 1c, 7, 8, 9, 10 |
| 1.152 | 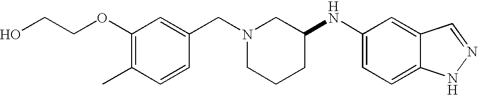 (S)-2-(5-((3-(1H-indazol-5-ylamino)piperidin-1- yl)methyl)-2-methylphenoxy)ethanol | 1c, 7, 8, 9, 10, 12a, 15c, 15d |
| 1.153 | 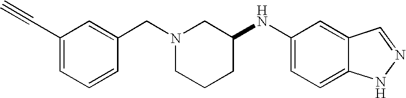 (S)-N-(1-(3-ethynylbenzyl)piperidin-3-yl)-1H-indazol-5- amine | 1c, 7, 8, 9, 10, 11, 14c |
| 1.154 | 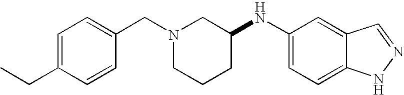 (S)-N-(1-(4-ethylbenzyl)piperidin-3-yl)-1H-indazol-5- amine | 1c, 7, 8, 9, 10 |
| 1.155 | 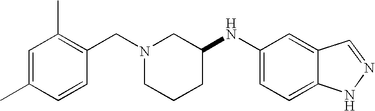 (S)-N-(1-(2,4-dimethylbenzyl)piperidin-3-yl)-1H- indazol-5-amine | 1c, 7, 8, 9, 10 |
| 1.156 | 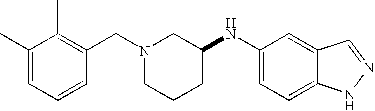 (S)-N-(1-(2,3-dimethylbenzyl)piperidin-3-yl)-1H- indazol-5-amine | 1c, 7, 8, 9, 10 |
| 1.157 | 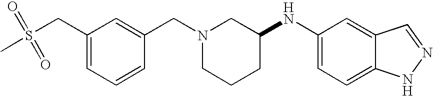 (S)-N-(1-(3-(methylsulfonylmethyl)benzyl)piperidin-3- yl)-1H-indazol-5-amine | 1c, 7, 8, 9, 10, 12, 15c |
| 1.158 | 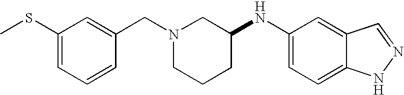 (S)-N-(1-(3-(methylthio)benzyl)piperidin-3-yl)-1H- indazol-5-amine | 1c, 7, 8, 9, 10, 12b, 15c, 15e |
| 1.159 | 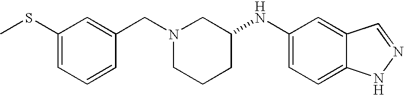 (R)-N-(1-(3-(methylthio)benzyl)piperidin-3-yl)-1H- indazol-5-amine | 1c, 7, 8, 9, 10, 12b, 15c, 15e |
| 1.160 | 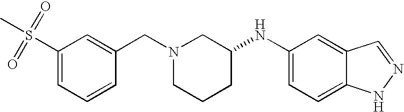 (R)-N-(1-(3-(methylsulfonyl)benzyl)piperidin-3-yl)-1H- indazol-5-amine | 1c, 7, 8, 9, 10, 12, 15c |
| 1.161 | 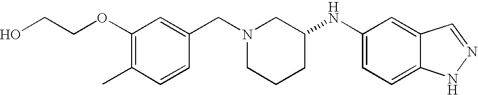 (R)-2-(5-((3-(1H-indazol-5-ylamino)piperidin-1- yl)methyl)-2-methylphenoxy)ethanol | 1c, 7, 8, 9, 10, 12a, 15c, 15d |
| 1.162 | 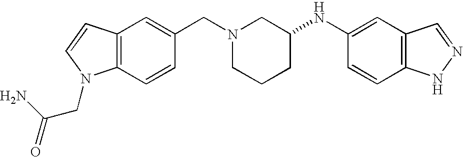 (R)-2-(5-((3-(1H-indazol-5-ylamino)piperidin-1- yl)methyl)-1H-indol-1-yl)acetamide | 1c, 7, 8, 9, 10, 13, 16c |
| 1.163 | 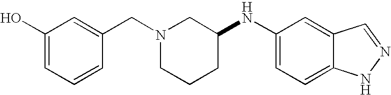 (S)-3-((3-(1H-indazol-5-ylamino)piperidin-1- yl)methyl)phenol | 1c, 7, 8, 9, 10 |
| 1.164 | 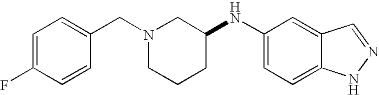 (S)-N-(1-(4-fluorobenzyl)piperidin-3-yl)-1H-indazol-5- amine | 1c, 7, 8, 9, 10 |
| 1.165 | 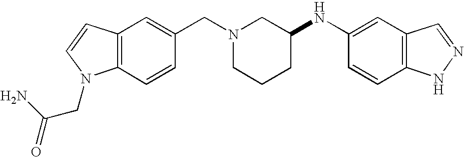 (S)-2-(5-((3-(1H-indazol-5-ylamino)piperidin-1- yl)methyl)-1H-indol-1-yl)acetamide | 1c, 7, 8, 9, 10, 13, 16c |
| 1.166 | 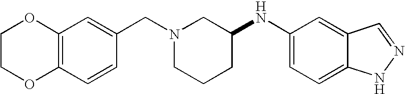 (S)-N-(1-((2,3-dihydrobenzo[b][1,4]dioxin-6- yl)methyl)piperidin-3-yl)-1H-indazol-5-amine | 1c, 7, 8, 9, 10 |
| 1.167 | 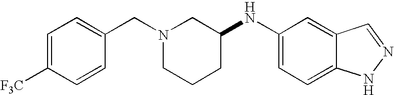 (S)-N-(1-(4-(trifluoromethyl)benzyl)piperidin-3-yl)-1H- indazol-5-amine | 1c, 7, 8, 9, 10 |
| 1.168 | 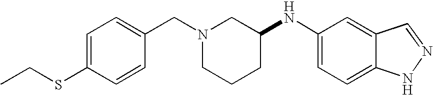 (S)-N-(1-(4-(ethylthio)benzyl)piperidin-3-yl)-1H- indazol-5-amine | 1c, 7, 8, 9, 10, 12b, 15c, 15e |
| 1.169 | 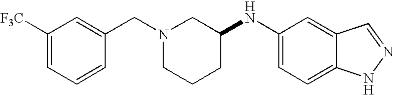 (S)-N-(1-(3-(trifluoromethyl)benzyl)piperidin-3-yl)-1H- indazol-5-amine | 1c, 7, 8, 9, 10 |
| 1.170 | 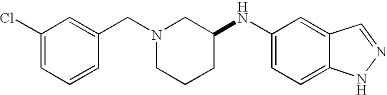 (S)-N-(1-(3-chlorobenzyl)piperidin-3-yl)-1H-indazol-5- amine | 1c, 7, 8, 9, 10 |
| 1.171 | 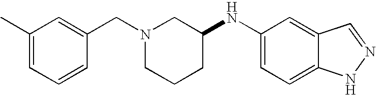 (S)-N-(1-(3-methylbenzyl)piperidin-3-yl)-1H-indazol-5- amine | 1.171 |
| 1.172 | 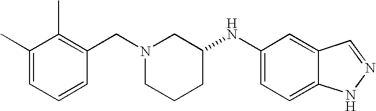 (R)-N-(1-(2,3-dimethylbenzyl)piperidin-3-yl)-1H- indazol-5-amine | 1.172 |
| 1.173 | 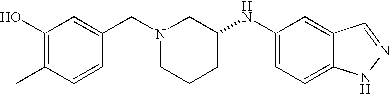 (R)-5-((3-(1H-indazol-5-ylamino)piperidin-1-yl)methyl)- 2-methylphenol | 1.173 |
| 1.174 | 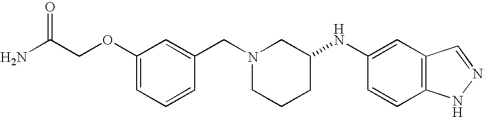 (R)-2-(3-((3-(1H-indazol-5-ylamino)piperidin-1- yl)methyl)phenoxy)acetamide | 1.174 |
| 1.175 | 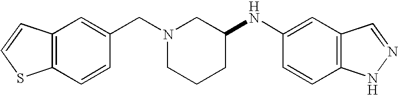 (S)-N-(1-(benzo[b]thiophen-5-ylmethyl)piperidin-3-yl)- 1H-indazol-5-amine | 1.175 |
| 1.176 | 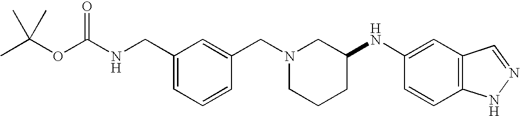 (S)-tert-butyl 3-((3-(1H-indazol-5-ylamino)piperidin-1- yl)methyl)benzylcarbamate | 1.176 |
| 1.177 | 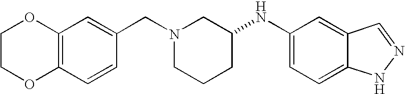 (R)-N-(1-((2,3-dihydrobenzo[b][1,4]dioxin-6- yl)methyl)piperidin-3-yl)-1H-indazol-5-amine | 1.177 |
| 1.178 | 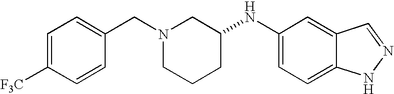 (R)-N-(1-(4-(trifluoromethyl)benzyl)piperidin-3-yl)-1H- indazol-5-amine | 1.178 |
| 1.179 | 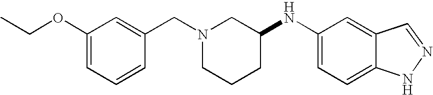 (S)-N-(1-(3-ethoxybenzyl)piperidin-3-yl)-1H-indazol-5- amine | 1.179 |
| 1.180 | 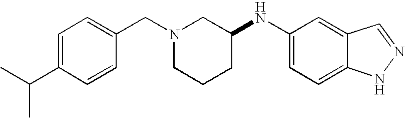 (S)-N-(1-(4-isopropylbenzyl)piperidin-3-yl)-1H-indazol- 5-amine | 1.180 |
| 1.181 | 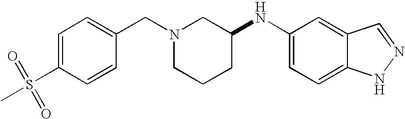 (S)-N-(1-(4-(methylsulfonyl)benzyl)piperidin-3-yl)-1H- indazol-5-amine | 1.181 |
| 1.182 | 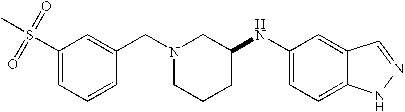 (S)-N-(1-(3-(methylsulfonyl)benzyl)piperidin-3-yl)-1H- indazol-5-amine | 1.182 |
| 1.183 | 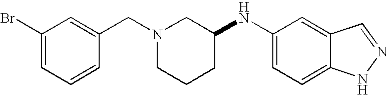 (S)-N-(1-(3-bromobenzyl)piperidin-3-yl)-1H-indazol-5- amine | 1.183 |
| 1.184 | 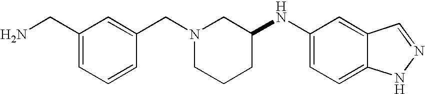 (S)-N-(1-(3-(aminomethyl)benzyl)piperidin-3-yl)-1H- indazol-5-amine | 1.184 |
| 1.185 | 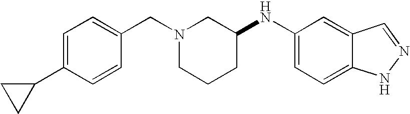 (S)-N-(1-(4-cyclopropylbenzyl)piperidin-3-yl)-1H- indazol-5-amine | 1.185 |
| 1.186 | 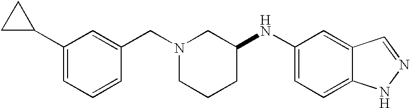 (S)-N-(1-(3-cyclopropylbenzyl)piperidin-3-yl)-1H- indazol-5-amine | 1.186 |
| 1.187 | 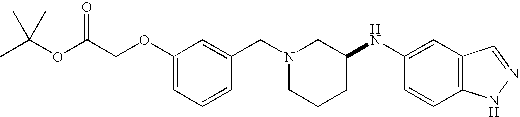 (S)-tert-butyl 2-(3-((3-(1H-indazol-5-ylamino)piperidin- 1-yl)methyl)phenoxy)acetate | 1.187 |
| 1.188 | 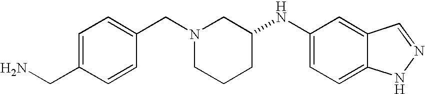 (R)-N-(1-(4-(aminomethyl)benzyl)piperidin-3-yl)-1H- indazol-5-amine | 1.188 |
| 1.189 | 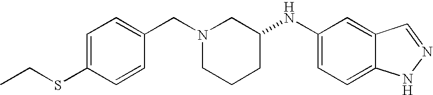 (R)-N-(1-(4-(ethylthio)benzyl)piperidin-3-yl)-1H- indazol-5-amine | 1.189 |
| 1.190 | 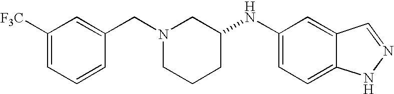 (R)-N-(1-(3-(trifluoromethyl)benzyl)piperidin-3-yl)-1H- indazol-5-amine | 1.190 |
| 1.191 | 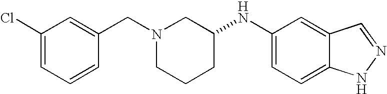 (R)-N-(1-(3-chlorobenzyl)piperidin-3-yl)-1H-indazol-5- amine | 1c, 7, 8, 9, 10 |
| 1.192 | 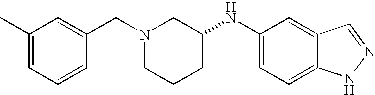 (R)-N-(1-(3-methylbenzyl)piperidin-3-yl)-1H-indazol-5- amine | 1c, 7, 8, 9, 10 |
| 1.193 | 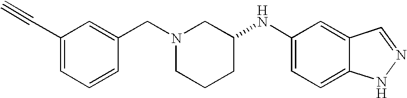 (R)-N-(1-(3-ethynylbenzyl)piperidin-3-yl)-1H-indazol-5- amine | 1c, 7, 8, 9, 10, 11, 14c |
| 1.194 | 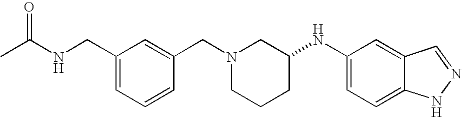 (R)-N-(1H-indazol-5-ylamino)piperidin-1- yl)methyl)benzyl)acetamide | 1c, 7, 8, 9, 10, 13, 16c |
| 1.195 | 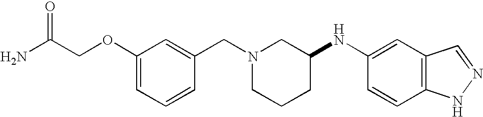 (S)-2-(3-((3-(1H-indazol-5-ylamino)piperidin-1- yl)methyl)phenyl-1H-indazol-5-ylamino)piperidin-1- yl)methyl)phenoxy)acetamide | 1c, 7, 8, 9, 10, 12a, 15c, 15d |
| 1.196 | 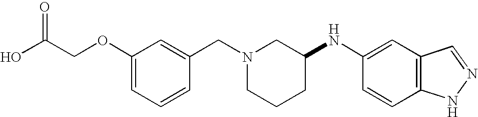 (S)-2-(3-((3-(1H-indazol-5-ylamino)piperidin-1- yl)methyl)phenoxy)acetic acid | 1c, 7, 8, 9, 10, 12a, 15c, 15d |
| 1.197 | 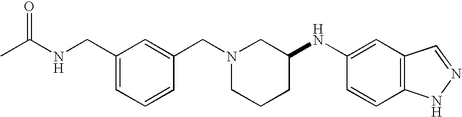 (S)-N-(1H-indazol-5-ylamino)piperidin-1- yl)methyl)benzyl)acetamide | 1c, 7, 8, 9, 10, 13, 16c |
| 1.198 | 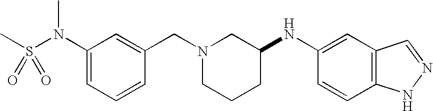 (S)-N-(3-((3-(1H-indazol-5-ylamino)piperidin-1- yl)methyl)phenyl)-N-methylmethanesulfonamide | 1c, 7, 8, 9, 10, 12a, 15c, 15d |
| 1.199 | 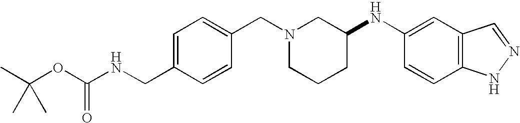 (S)-tert-butyl 4-((3-(1H-indazol-5-ylamino)piperidin-1- yl)methyl)benzylcarbamate | 1c, 7, 8, 9, 10, 13, 16c |
| 1.200 | 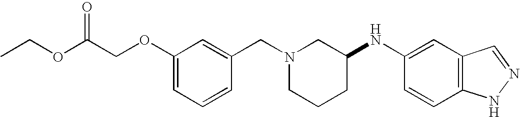 (S)-ethyl 2-(3-((3-(1H-indazol-5-ylamino)piperidin-1- yl)methyl)phenoxy)acetate | 1c, 7, 8, 9, 10, 12a, 15c, 15d |
| 1.201 | 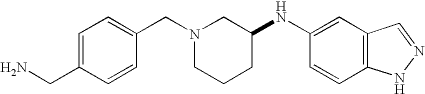 (S)-N-(1-(4-(aminomethyl)benzyl)piperidin-3-yl)-1H- indazol-5-amine | 1c, 7, 8, 9, 10, 13, 16c |
| 1.202 | 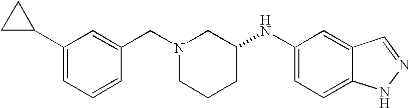 (R)-N-(1-(3-cyclopropylbenzyl)piperidin-3-yl)-1H- indazol-5-amine | 1c, 7, 8, 9, 10, 11, 14c |
| 1.203 | 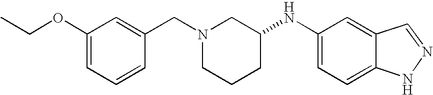 (R)-N-(1-(3-ethoxybenzyl)piperidin-3-yl)-1H-indazol-5- amine | 1c, 7, 8, 9, 10 |
| 1.204 | 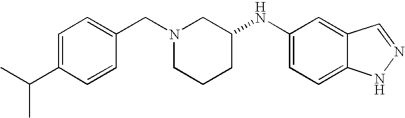 (R)-N-(1-(4-isopropylbenzyl)piperidin-3-yl)-1H-indazol- 5-amine | 1c, 7, 8, 9, 10 |
| 1.205 | 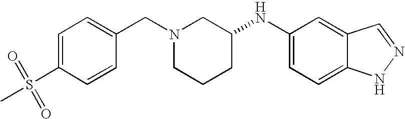 (R)-N-(1-(4-(methylsulfonyl)benzyl)piperidin-3-yl)-1H- indazol-5-amine | 1c, 7, 8, 9, 10, 12, 15c |
| 1.206 | 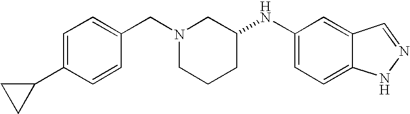 (R)-N-(1-(4-cyclopropylbenzyl)piperidin-3-yl)-1H- indazol-5-amine | 1c, 7, 8, 9, 10, 11, 14c |
| 1.207 | 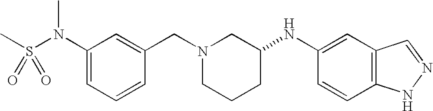 (R)-N-(3-((3-(1H-indazol-5-ylamino)piperidin-1- yl)methyl)phenyl)-N-methylmethanesulfonamide | 1c, 7, 8, 9, 10, 12a, 15c, 15d |
| 1.208 | 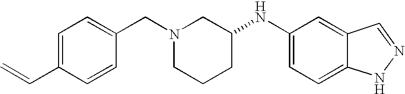 (R)-N-(1-(4-vinylbenzyl)piperidin-3-yl)-1H-indazol-5- amine | 1c, 7, 8, 9, 10, 11, 14c |
| 1.209 | 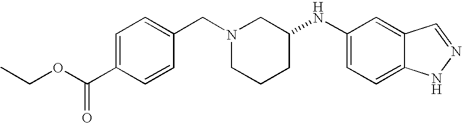 (R)-ethyl 4-((3-(1H-indazol-5-ylamino)piperidin-1- yl)methyl)benzoate | 1c, 7, 8, 9, 10 |
| 1.210 | 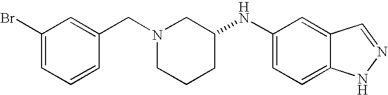 (R)-N-(1-(3-bromobenzyl)piperidin-3-yl)-1H-indazol-5- amine | 1c, 7, 8, 9, 10 |
| 1.211 | 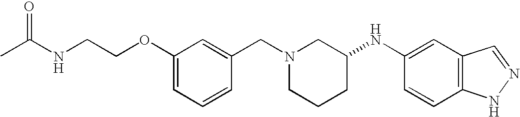 (R)-N-(2-(3-((3-(1H-indazol-5-ylamino)piperidin-1- yl)methyl)phenoxy)ethyl)acetamide | 1c, 7, 8, 9, 10, 12a, 15c, 15d |
| 1.212 | 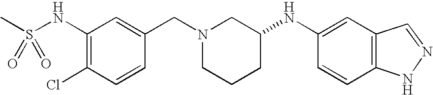 (R)-N-(5-((3-(1H-indazol-5-ylamino)piperidin-1- yl)methyl)-2-chlorophenyl)methanesulfonamide | 1c, 7, 8, 9, 10, 12a, 15c, 15d |
| 1.213 | 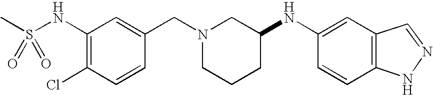 (S)-N-(5-((3-(1H-indazol-5-ylamino)piperidin-1- yl)methyl)-2-chlorophenyl)methanesulfonamide | 1c, 7, 8, 9, 10, 12a, 15c, 15d |
| 1.214 | 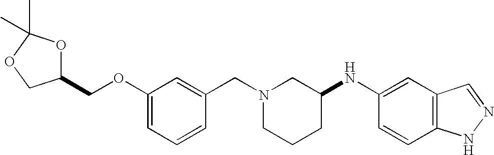 N-((S)-1-(3-(((S)-2,2-dimethyl-1,3-dioxolan-4- yl)methoxy)benzyl)piperidin-3-yl)-1H-indazol-5-amine | 1c, 7, 8, 9, 10, 12a, 15c, 15d |
| 1.215 | 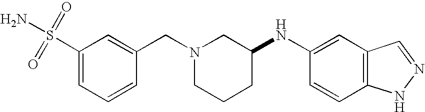 (S)-3-((3-(1H-indazol-5-ylamino)piperidin-1- yl)methyl)benzenesulfonamide | 1c, 7, 8, 9, 10, 12, 15c |
| 1.216 | 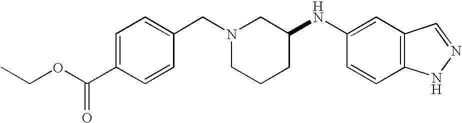 (S)-ethyl 4-((3-(1H-indazol-5-ylamino)piperidin-1- yl)methyl)benzoate | 1c, 7, 8, 9, 10 |
| 1.217 | 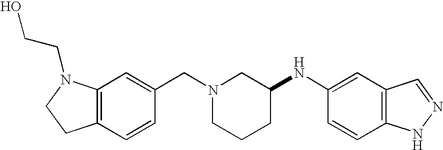 (S)-2-(6-((3-(1H-indazol-5-ylamino)piperidin-1- yl)methyl)indolin-1-yl)ethanol | 1c, 7, 8, 9, 10, 13, 16c |
| 1.218 | 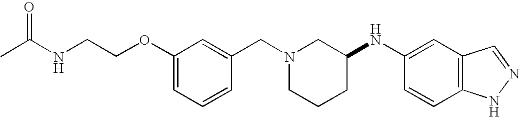 (S)-N-(2-(3-((3-(1H-indazol-5-ylamino)piperidin-1- yl)methyl)phenoxy)ethyl)acetamide | 1c, 7, 8, 9, 10, 12a, 15c, 15d |
| 1.219 | 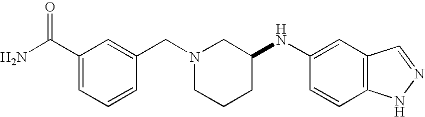 (S)-3-((3-(1H-indazol-5-ylamino)piperidin-1- yl)methyl)benzamide | 1c, 7, 8, 9, 10, 12, 15c |
| 1.221 | 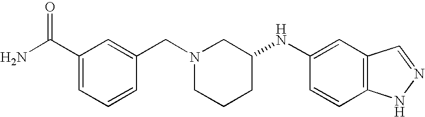 (R)-3-((3-(1H-indazol-5-ylamino)piperidin-1- yl)methyl)benzamide | 1c, 7, 8, 9, 10, 12, 15c |
| 1.222 | 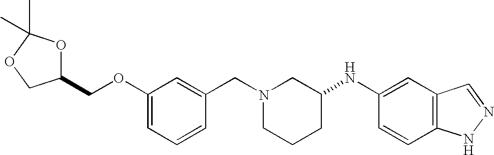 N-((R)-1-(3-(((S)-2,2-dimethyl-1,3-dioxolan-4- yl)methoxy)benzyl)piperidin-3-yl)-1H-indazol-5-amine | 1c, 7, 8, 9, 10, 12a, 15c, 15d |
| 1.223 | 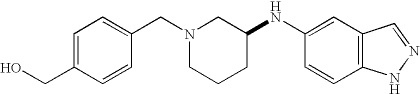 (S)-(4-((3-(1H-indazol-5-ylamino)piperidin-1- yl)methyl)phenyl)methanol | 1c, 7, 8, 9, 10, 13, 16c |
| 1.224 | 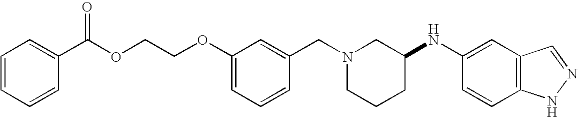 (S)-2-(3-((3-(1H-indazol-5-ylamino)piperidin-1- yl)methyl)phenoxy)ethyl benzoate | 1c, 7, 8, 9, 10, 12a, 15c, 15d |
| 1.225 | 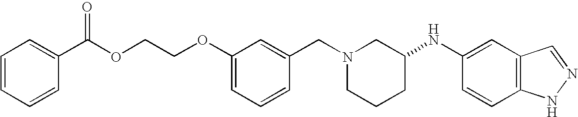 (R)-2-(3-((3-(1H-indazol-5-ylamino)piperidin-1- yl)methyl)phenoxy)ethyl benzoate | 1c, 7, 8, 9, 10, 12a, 15c, 15d |
| 1.226 | 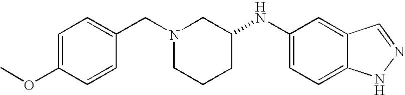 (R)-N-(1-(4-methoxybenzyl)piperidin-3-yl)-1H-indazol- 5-amine | 1c, 7, 8, 9, 10 |
| 1.227 | 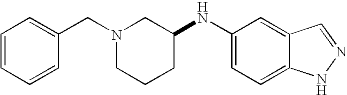 (S)-N-(1-benzylpiperidin-3-yl)-1H-indazol-5-amine | 1c, 7, 8, 9, 10 |
| 1.228 | 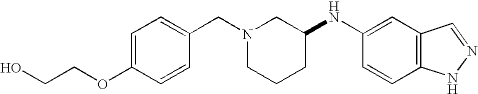 (S)-2-(4-((3-(1H-indazol-5-ylamino)piperidin-1- yl)methyl)phenoxy)ethanol | 1c, 7, 8, 9, 10, 12a, 15c, 15d |
| 1.229 | 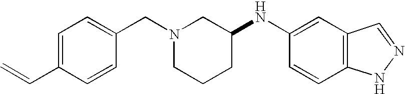 (S)-N-(1-(4-vinylbenzyl)piperidin-3-yl)-1H-indazol-5- amine | 1c, 7, 8, 9, 10, 11, 14c |
| 1.230 | 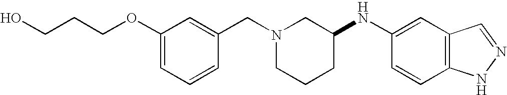 (S)-3-(3-((3-(1H-indazol-5-ylamino)piperidin-1- yl)methyl)phenoxy)propan-1-ol | 1c, 7, 8, 9, 10, 12a, 15c, 15d |
| 1.231 | 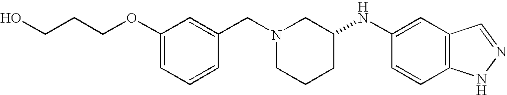 (R)-3-(3-((3-(1H-indazol-5-ylamino)piperidin-1- yl)methyl)phenoxy)propan-1-ol | 1c, 7, 8, 9, 10, 12a, 15c, 15d |
| 1.232 | 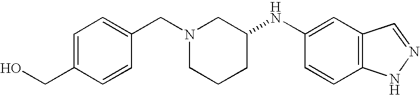 (R)-(4-((3-(1H-indazol-5-ylamino)piperidin-1- yl)methyl)phenyl)methanol | 1c, 7, 8, 9, 10 |
| 1.233 | 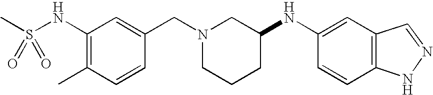 (S)-N-(5-((3-(1H-indazol-5-ylamino)piperidin-1- yl)methyl)-2-methylphenyl)methanesulfonamide | 1c, 7, 8, 9, 10, 12a, 15c, 15d |
| 1.234 | 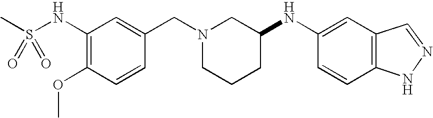 (S)-N-(5-((3-(1H-indazol-5-ylamino)piperidin-1- yl)methyl)-2-methoxyphenyl)methanesulfonamide | 1c, 7, 8, 9, 10, 12a, 15c, 15d |
| 1.235 | 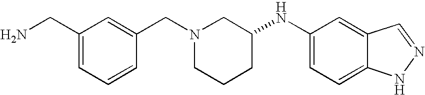 (R)-N-(1-(3-(aminomethyl)benzyl)piperidin-3-yl)-1H- indazol-5-amine | 1c, 7, 8, 9, 10, 13, 16c |
| 1.236 | 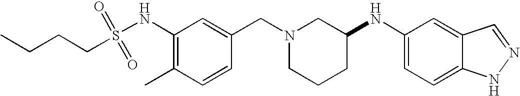 (S)-N-(5-((3-(1H-indazol-5-ylamino)piperidin-1- yl)methyl)-2-methylphenyl)butane-1-sulfonamide | 1c, 7, 8, 9, 10, 12a, 15c, 15d |
| 1.237 | 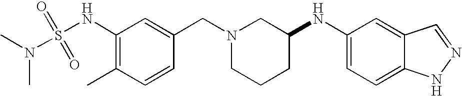 (S)-N-(2-((3-(1H-indazol-5-ylamino)piperidin-1- yl)methyl)-5-methylphenyl)-N′,N′ dimethylaminosulfamide | 1c, 7, 8, 9, 10, 12a, 15c, 15d |
| 1.238 | 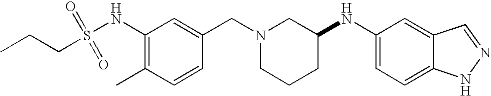 (S)-N-(5-((3-(1H-indazol-5-ylamino)piperidin-1- yl)methyl)-2-methylphenyl)propane-1-sulfonamide | 1c, 7, 8, 9, 10, 12a, 15c, 15d |
| 1.239 | 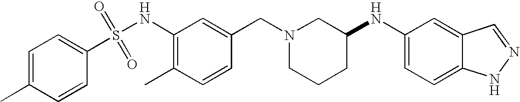 (S)-N-(5-((3-(1H-indazol-5-ylamino)piperidin-1- yl)methyl)-2-methylphenyl)-4- methylbenzenesulfonamide | 1c, 7, 8, 9, 10, 12a, 15c, 15d |
| 1.240 | 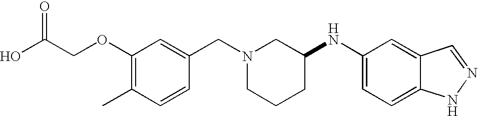 (S)-2-(5-((3-(1H-indazol-5-ylamino)piperidin-1- yl)methyl)-2-methylphenyl-1H-indazol-5- ylamino)piperidin-1-yl)methyl)-2-methylphenoxy)acetic acid | 1c, 7, 8, 9, 10, 12a, 15c, 15d |
| 1.241 | 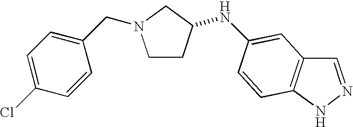 (R)-N-(1-(4-chlorobenzyl)pyrrolidin-3-yl)-1H-indazol-5- amine | 1c, 7, 8, 9, 10 |
| 1.242 | 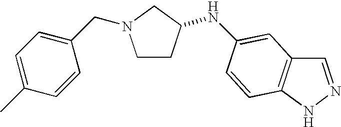 (R)-N-(1-(4-methylbenzyl)pyrrolidin-3-yl)-1H-indazol-5- amine | 1c, 7, 8, 9, 10 |
| 1.243 | 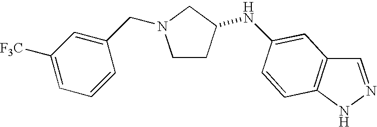 (R)-N-(1-(3-(trifluoromethyl)benzyl)pyrrolidin-3-yl)-1H- indazol-5-amine | 1c, 7, 8, 9, 10 |
| 1.244 | 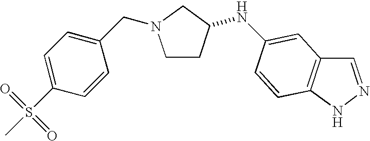 (R)-N-(1-(4-(methylsulfonyl)benzyl)pyrrolidin-3-yl)-1H- indazol-5-amine | 1c, 7, 8, 9, 10, 12b, 15c, 15e |
| 1.245 | 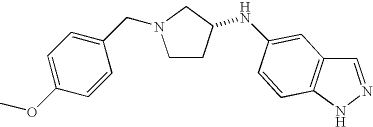 (R)-N-(1-(4-methoxybenzyl)pyrrolidin-3-yl)-1H-indazol- 5-amine | 1c, 7, 8, 9, 10 |
| 1.246 | 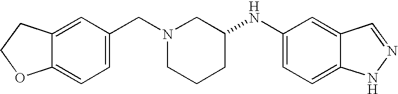 (R)-N-(1-((2,3-dihydrobenzofuran-5- yl)methyl)piperidin-3-yl)-1H-indazol-5-amine | 1c, 7, 8, 9, 10 |
| 1.247 | 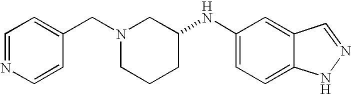 (R)-N-(1-(pyridin-4-ylmethyl)piperidin-3-yl)-1H-indazol- 5-amine | 1c, 7, 8, 9, 10 |
| 1.248 | 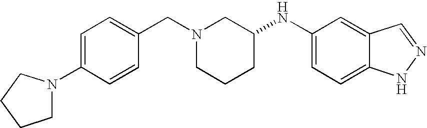 (R)-N-(1-(4-(pyrrolidin-1-yl)benzyl)piperidin-3-yl)-1H- indazol-5-amine | 1c, 7, 8, 9, 10, 11, 14c |
| 1.249 | 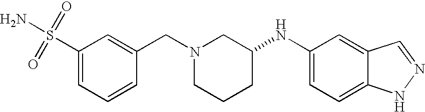 (R)-3-((3-(1H-indazol-5-ylamino)piperidin-1- yl)methyl)benzenesulfonamide | 1c, 7, 8, 9, 10, 12b, 15c, 15e |
| 1.250 | 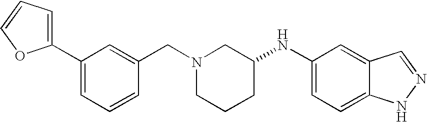 (R)-N-(1-(3-(furan-2-yl)benzyl)piperidin-3-yl)-1H- indazol-5-amine | 1c, 7, 8, 9, 10, 11, 14c |
| 1.251 | 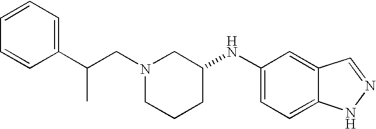 N-((3R)-1-(2-phenylpropyl)piperidin-3-yl)-1H-indazol- 5-amine | 1c, 7, 8, 9 |
| 1.252 | 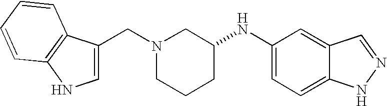 (R)-N-(1-((1H-indol-3-yl)methyl)piperidin-3-yl)-1H- indazol-5-amine | 1c, 7, 8, 9, 10 |
| 1.253 | 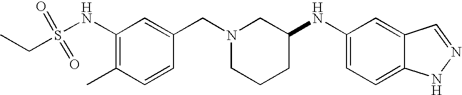 (S)-N-(5-((3-(1H-indazol-5-ylamino)piperidin-1- yl)methyl)-2-methylphenyl)ethanesulfonamide | 1c, 7, 8, 9, 10, 12a, 15c, 15d |
| 1.254 | 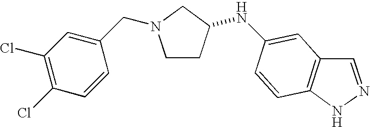 (R)-N-(1-(3,4-dichlorobenzyl)pyrrolidin-3-yl)-1H- indazol-5-amine | 1c, 7, 8, 9, 10 |
| 1.255 | 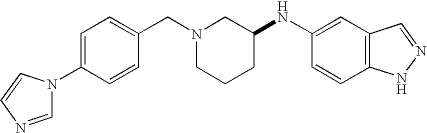 (S)-N-(1-(1H-imidazol-1-yl)benzyl)piperidin-3-yl)-1H- indazol-5-amine | 1c, 7, 8, 9, 10, 11, 14c |
| 1.256 | 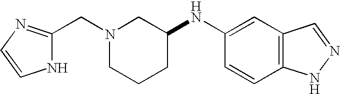 (S)-N-(1-((1H-imidazol-2-yl)methyl)piperidin-3-yl)-1H- indazol-5-amine | 1c, 7, 8, 9, 10 |
| 1.257 | 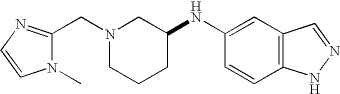 (S)-N-(1-((1-methyl-1H-imidazol-2-yl)methyl)piperidin- 3-yl)-1H-indazol-5-amine | 1c, 7, 8, 9, 10 |
| 1.258 | 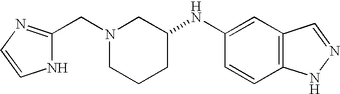 (R)-N-(5-((3-(1H-indazol-5-ylamino)piperidin-1- yl)methyl)-2-methylphenyl)methanesulfonamide | 1c, 7, 8, 9, 10, 12a, 15c, 15d |
| 1.259 | 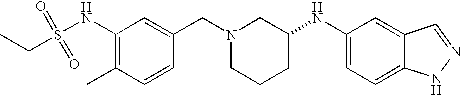 (R)-N-(5-((3-(1H-indazol-5-ylamino)piperidin-1- yl)methyl)-2-methylphenyl)ethanesulfonamide | 1c, 7, 8, 9, 10, 12a, 15c, 15d |
| 1.260 | 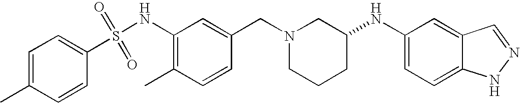 (R)-N-(5-((3-(1H-indazol-5-ylamino)piperidin-1- yl)methyl)-2-methylphenyl)-4- methylbenzenesulfonamide | 1c, 7, 8, 9, 10, 12a, 15c, 15d |
| 1.261 | 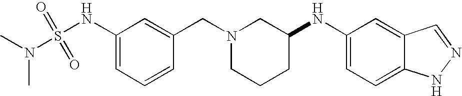 (S)-N-(3-((3-(1H-indazol-5-ylamino)piperidin-1- yl)methyl)phenyl)-N′,N′ dimethylaminosulfamide | 1c, 7, 8, 9, 10, 12a, 15c, 15d |
| 1.262 | 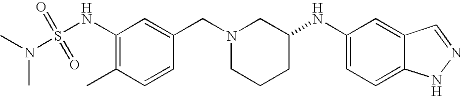 (R)-N-(2-((3-(1H-indazol-5-ylamino)piperidin-1- yl)methyl)-5-methylphenyl)-N′,N′ dimethylaminosulfamide | 1c, 7, 8, 9, 10, 12a, 15c, 15d |
| 1.263 | 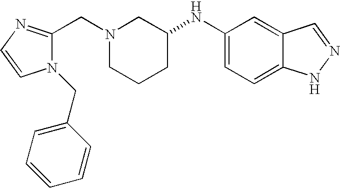 (R)-N-(1-((1-benzyl-1H-imidazol-2-yl)methyl)piperidin- 3-yl)-1H-indazol-5-amine | 1c, 7, 8, 9, 10, 11, 14c |
| 1.264 | 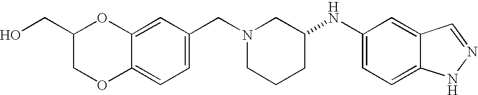 (7-(((R)-3-(1H-indazol-5-ylamino)piperidin-1- yl)methyl)-2,3-dihydrobenzo[b][1,4]dioxin-2- yl)methanol | 1c, 7, 8, 9, 10, 13, 16c |
| 1.265 | 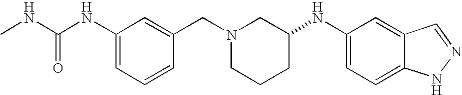 (R)-1-(3-((3-(1H-indazol-5-ylamino)piperidin-1- yl)methyl)phenyl)-3-methylurea | 1c, 7, 8, 9, 10, 12a, 15c, 15d |
| 1.266 | 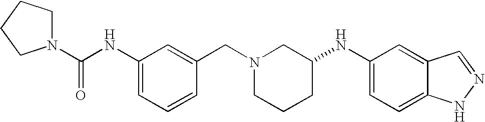 (R)-N-(3-((3-(1H-indazol-5-ylamino)piperidin-1- yl)methyl)phenyl)pyrrolidine-1-carboxamide | 1c, 7, 8, 9, 10, 12a, 15c, 15d |
| 1.267 | 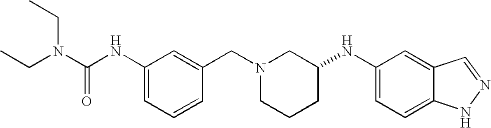 (R)-3-(3-((3-(1H-indazol-5-ylamino)piperidin-1- yl)methyl)phenyl)-1,1-diethylurea | 1c, 7, 8, 9, 10, 12a, 15c, 15d |
| 1.268 | 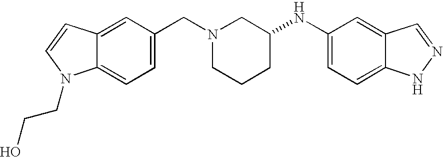 (R)-2-(5-((3-(1H-indazol-5-ylamino)piperidin-1- yl)methyl)-1H-indol-1-yl)ethanol | 1c, 7, 8, 9, 10, 13, 16c |
| 1.269 | 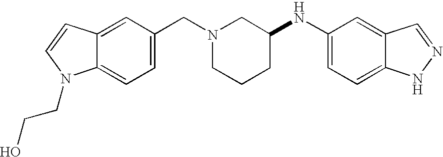 (S)-2-(5-((3-(1H-indazol-5-ylamino)piperidin-1- yl)methyl)-1H-indol-1-yl)ethanol | 1c, 7, 8, 9, 10, 13, 16c |
| 1.270 | 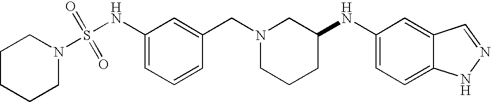 (S)-N-(3-((3-(1H-indazol-5-ylamino)piperidin-1- yl)methyl)phenyl)piperidine-1-sulfonamide | 1c, 7, 8, 9, 10, 12a, 15c, 15d |
| 1.271 | 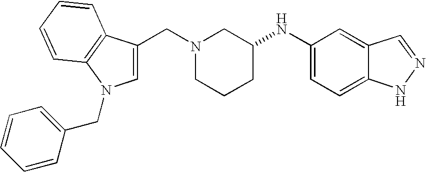 (R)-N-(1-((1-benzyl-1H-indol-3-yl)methyl)piperidin-3- yl)-1H-indazol-5-amine | 1c, 7, 8, 9, 10, 11, 14c |
| 1.272 | 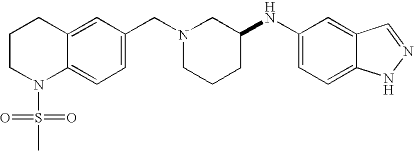 (S)-N-(1-((1-(methylsulfonyl)-1,2,3,4- tetrahydroquinolin-6-yl)methyl)piperidin-3-yl)-1H- indazol-5-amine | 1c, 7, 8, 9, 10, 12b, 15c, 15e |
| 1.273 | 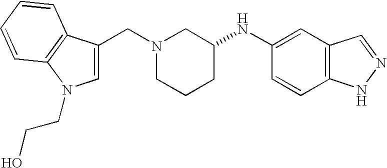 (R)-2-(3-((3-(1H-indazol-5-ylamino)piperidin-1- yl)methyl)-1H-indol-1-yl)ethanol | 1c, 7, 8, 9, 10, 13, 16c |
| 1.274 | 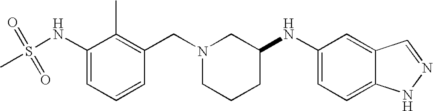 (S)-N-(3-((3-(1H-indazol-5-ylamino)piperidin-1- yl)methyl)-2-methylphenyl)methanesulfonamide | 1c, 7, 8, 9, 10, 12a, 15c, 15d |
| 1.275 | 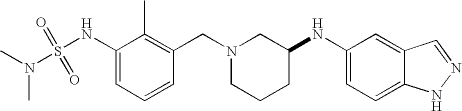 (S)-N-(3-((3-(1H-indazol-5-ylamino)piperidin-1- yl)methyl)-2-methylphenyl)-N′,N′ dimethylaminosulfamide | 1c, 7, 8, 9, 10, 12a, 15c, 15d |
| 1.276 | 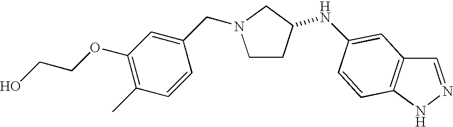 (R)-2-(5-((3-(1H-indazol-5-ylamino)pyrrolidin-1- yl)methyl)-2-methylphenyl-1H-indazol-5- ylamino)pyrrolidin-1-yl)methyl)-2- methylphenoxy)ethanol | 1c, 7, 8, 9, 10, 12a, 15c, 15d |
| 1.277 | 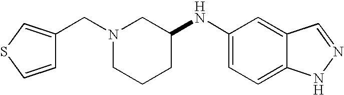 (S)-N-(1-(thiophen-3-ylmethyl)piperidin-3-yl)-1H- indazol-5-amine | 1c, 7, 8, 9, 10 |
| 1.278 | 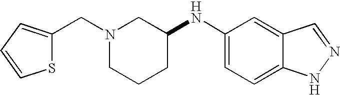 (S)-N-(1-(thiophen-2-ylmethyl)piperidin-3-yl)-1H- indazol-5-amine | 1c, 7, 8, 9, 10 |
| 1.279 | 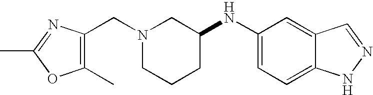 (S)-N-(1-((2,5-dimethyloxazol-4-yl)methyl)piperidin-3- yl)-1H-indazol-5-amine | 1c, 7, 8, 9, 10 |
| 1.280 | 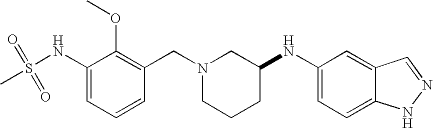 (S)-N-(3-((3-(1H-indazol-5-ylamino)piperidin-1- yl)methyl)-2-methoxyphenyl)methanesulfonamide | 1c, 7, 8, 9, 10, 12a, 15c, 15d |
| 1.281 | 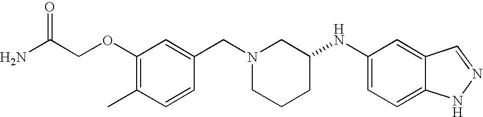 (R)-2-(5-((3-(1H-indazol-5-ylamino)piperidin-1- yl)methyl)-2-methylphenyl-1H-indazol-5- ylamino)piperidin-1-yl)methyl)-2- methylphenoxy)acetamide | 1c, 7, 8, 9, 10, 12a, 15c, 15d |
| 1.282 | 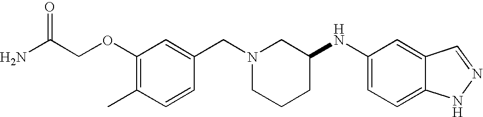 (S)-2-(5-((3-(1H-indazol-5-ylamino)piperidin-1- yl)methyl)-2-methylphenyl-1H-indazol-5- ylamino)piperidin-1-yl)methyl)-2- methylphenoxy)acetamide | 1c, 7, 8, 9, 10, 12a, 15c, 15d |
| 2.001 | 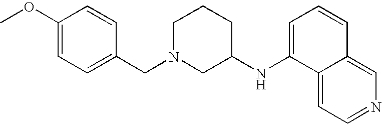 N-(1-(4-methoxybenzyl)piperidin-3-yl)isoquinolin-5- amine | 2c, 7, 8, 9, 10 |
| 2.002 | 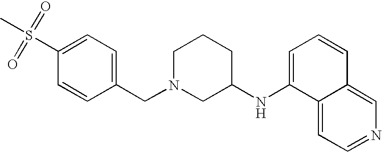 N-(1-(4-(methylsulfonyl)benzyl)piperidin-3- yl)isoquinolin-5-amine | 2c, 7, 8, 9, 10, 12, 15c |
| 2.003 | 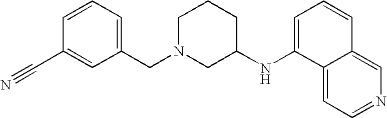 3-((3-(isoquinolin-5-ylamino)piperidin-1- yl)methyl)benzonitrile | 2c, 7, 8, 9, 10, 12, 15c |
| 2.004 | 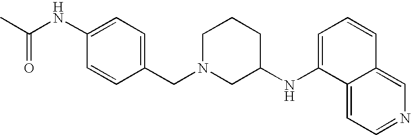 N-(4-((3-(isoquinolin-5-ylamino)piperidin-1- yl)methyl)phenyl)acetamide | 2c, 7, 8, 9, 10, 15c, 15d |
| 2.005 | 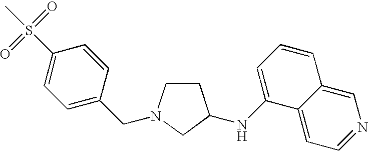 N-(1-(4-(methylsulfonyl)benzyl)pyrrolidin-3- yl)isoquinolin-5-amine | 2c, 7, 8, 9, 10, 12, 15c |
| 2.006 | 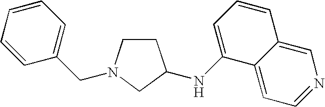 N-(1-benzylpyrrolidin-3-yl)isoquinolin-5-amine | 2c, 7, 8, 9, 10 |
| 2.007 | 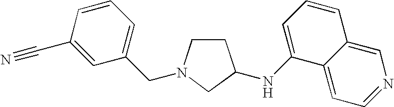 3-((3-(isoquinolin-5-ylamino)pyrrolidin-1- yl)methyl)benzonitrile | 2c, 7, 8, 9, 10, 12, 15c |
| 2.008 | 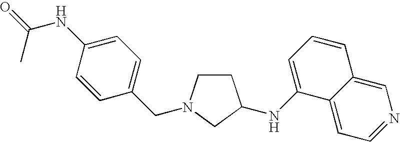 N-(4-((3-(isoquinolin-5-ylamino)pyrrolidin-1- yl)methyl)phenyl)acetamide | 2c, 7, 8, 9, 10, 12a, 15c, 15d |
| 2.009 | 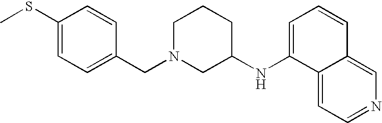 N-(1-(4-(methylthio)benzyl)piperidin-3-yl)isoquinolin-5- amine | 2c, 7, 8, 9, 10, 12b, 15c, 15e |
| 2.010 | 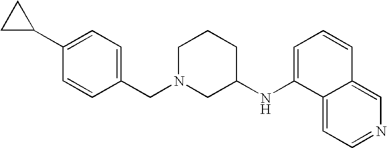 N-(1-(4-cyclopropylbenzyl)piperidin-3-yl)isoquinolin-5- amine | 2c, 7, 8, 9, 10, 11, 14c |
| 2.011 | 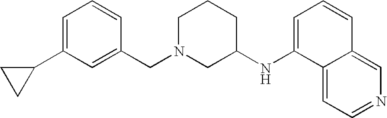 N-(1-(3-cyclopropylbenzyl)piperidin-3-yl)isoquinolin-5- amine | 2c, 7, 8, 9, 10, 11, 14c |
| 2.012 | 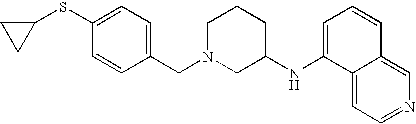 N-(1-(4-(cyclopropylthio)benzyl)piperidin-3- yl)isoquinolin-5-amine | 2c, 7, 8, 9, 10, 12b, 15c, 15e |
| 2.013 | 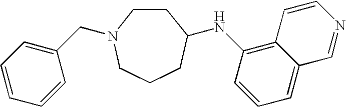 N-(1-benzylazepan-4-yl)isoquinolin-5-amine | 2c, 7, 8, 9, 10 |
| 2.014 | 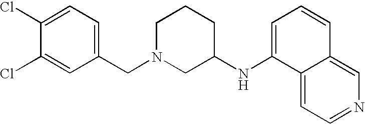 N-(1-(3,4-dichlorobenzyl)piperidin-3-yl)isoquinolin-5- amine | 2c, 7, 8, 9, 10 |
| 2.015 | 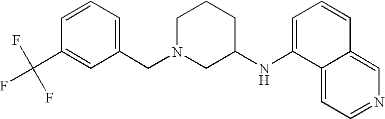 N-(1-(3-(trifluoromethyl)benzyl)piperidin-3- yl)isoquinolin-5-amine | 2c, 7, 8, 9, 10 |
| 2.016 | 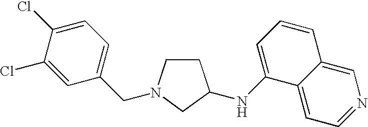 N-(1-(3,4-dichlorobenzyl)pyrrolidin-3-yl)isoquinolin-5- amine | 2c, 7, 8, 9, 10 |
| 2.017 | 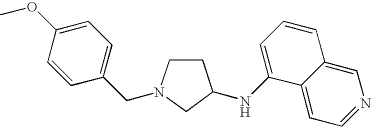 N-(1-(4-methoxybenzyl)pyrrolidin-3-yl)isoquinolin-5- amine | 2c, 7, 8, 9, 10 |
| 2.018 | 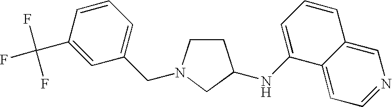 N-(1-(3-(trifluoromethyl)benzyl)pyrrolidin-3- yl)isoquinolin-5-amine | 2c, 7, 8, 9, 10 |
| 2.019 | 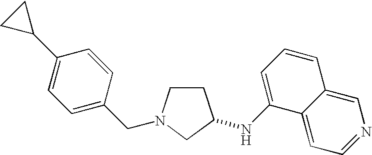 (S)-N-(1-(4-cyclopropylbenzyl)pyrrolidin-3- yl)isoquinolin-5-amine | 2c, 7, 8, 9, 10, 11, 14c |
| 2.020 | 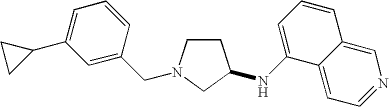 (R)-N-(1-(3-cyclopropylbenzyl)pyrrolidin-3- yl)isoquinolin-5-amine | 2c, 7, 8, 9, 10, 11, 14c |
| 2.021 | 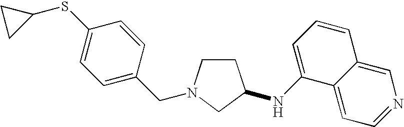 (R)-N-(1-(4-(cyclopropylthio)benzyl)pyrrolidin-3- yl)isoquinolin-5-amine | 2c, 7, 8, 9, 10, 12b, 15c, 15e |
| 2.022 | 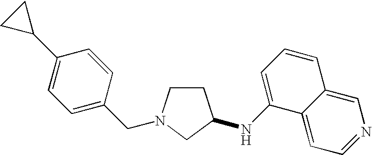 (R)-N-(1-(4-cyclopropylbenzyl)pyrrolidin-3- yl)isoquinolin-5-amine | 2c, 7, 8, 9, 10, 11, 14c |
| 2.023 | 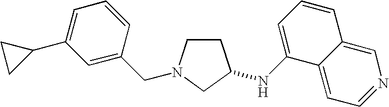 (S)-N-(1-(3-cyclopropylbenzyl)pyrrolidln-3- yl)isoquinolin-5-amine | 2c, 7, 8, 9, 10, 11, 14c |
| 2.024 | 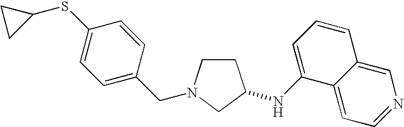 (S)-N-(1-(4-(cyclopropylthio)benzyl)pyrrolidin-3- yl)isoquinolin-5-amine | 2c, 7, 8, 9, 10, 12b, 15c, 15e |
| 2.025 | 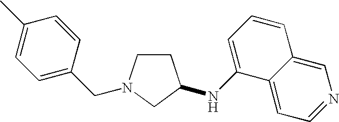 (R)-N-(1-(4-methylbenzyl)pyrrolidin-3-yl)isoquinolin-5- amine | 2c, 7, 8, 9, 10 |
| 2.026 | 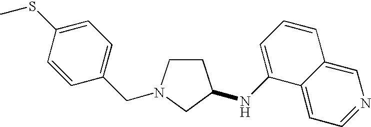 (R)-N-(1-(4-(methylthio)benzyl)pyrrolidin-3- yl)isoquinolin-5-amine | 2c, 7, 8, 9, 10, 12b, 15c, 15e |
| 2.027 | 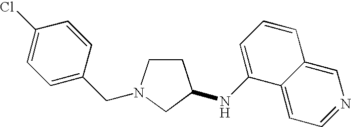 (R)-N-(1-(4-chlorobenzyl)pyrrolidin-3-yl)isoquinolin-5- amine | 2c, 7, 8, 9, 10 |
| 2.028 | 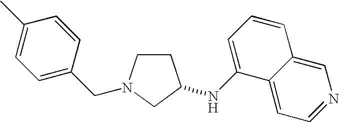 (S)-N-(1-(4-methylbenzyl)pyrrolidin-3-yl)isoquinolin-5- amine | 2c, 7, 8, 9, 10 |
| 2.029 | 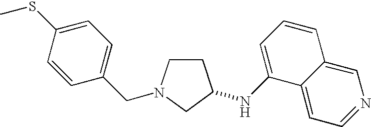 (S)-N-(1-(4-(methylthio)benzyl)pyrrolidin-3- yl)isoquinolin-5-amine | 2c, 7, 8, 9, 10, 12b, 15c, 15e |
| 2.030 | 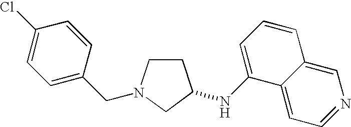 (S)-N-(1-(4-chlorobenzyl)pyrrolidin-3-yl)isoquinolin-5- amine | 2c, 7, 8, 9, 10 |
| 2.031 | 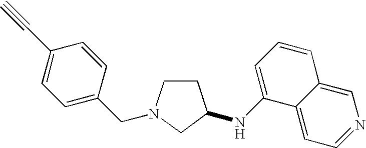 (R)-N-(1-(4-ethynylbenzyl)pyrrolidin-3-yl)isoquinolin-5- amine | 2c, 7, 8, 9, 10, 11, 14c |
| 2.032 | 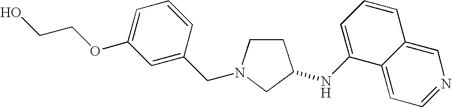 (S)-2-(3-((3-(isoquinolin-5-ylamino)pyrrolidin-1- yl)methyl)phenoxy)ethanol | 2c, 7, 8, 9, 10, 12a, 15c, 15d |
| 2.033 | 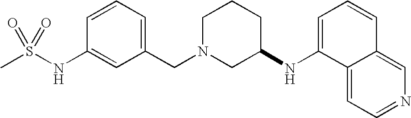 (R)-N-(3-((3-(isoquinolin-5-ylamino)piperidin-1- yl)methyl)phenyl)methanesulfonamide | 2c, 7, 8, 9, 10, 12a, 15c, 15d |
| 2.034 | 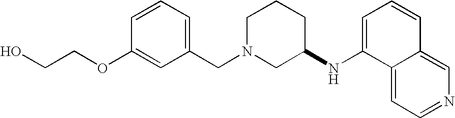 (R)-2-(3-((3-(isoquinolin-5-ylamino)piperidin-1- yl)methyl)phenoxy)ethanol | 2c, 7, 8, 9, 10, 12a 15c, 15d |
| 2.035 | 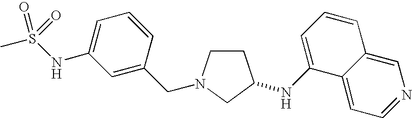 (S)-N-(3-((3-(isoquinolin-5-ylamino)pyrrolidin-1- yl)methyl)phenyl)methanesulfonamide | 2c, 7, 8, 9, 10, 12a, 15c, 15d |
| 2.036 | 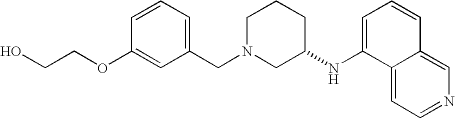 (S)-2-(3-((3-(isoquinolin-5-ylamino)piperidin-1- yl)methyl)phenoxy)ethanol | 2c, 7, 8, 9, 10, 12a, 15c, 15d |
| 2.037 | 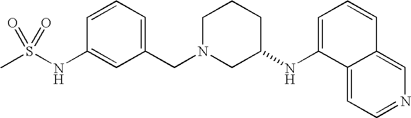 (S)-N-(3-((3-(isoquinolin-5-ylamino)piperidin-1- yl)methyl)phenyl)methanesulfonamide | 2c, 7, 8, 9, 10, 12a, 15c, 15d |
| 2.038 | 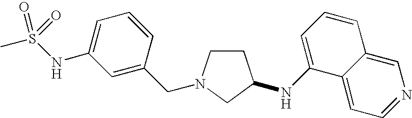 (R)-N-(3-((3-(isoquinolin-5-ylamino)pyrrolidin-1- yl)methyl)phenyl)methanesulfonamide | 2c, 7, 8, 9, 10, 12a, 15c, 15d |
| 2.039 | 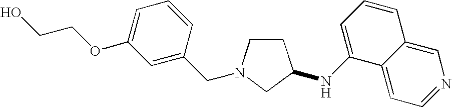 (R)-2-(3-((3-(isoquinolin-5-ylamino)pyrrolidin-1- yl)methyl)phenoxy)ethanol | 2c, 7, 8, 9, 10, 12a, 15c, 15d |
| 2.040 | 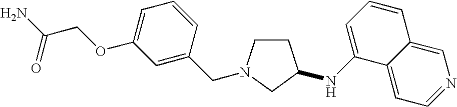 (R)-2-(3-((3-(isoquinolin-5-ylamino)pyrrolidin-1- yl)methyl)phenoxy)acetamide | 2c, 7, 8, 9, 10, 12a, 15c, 15d |
| 2.041 | 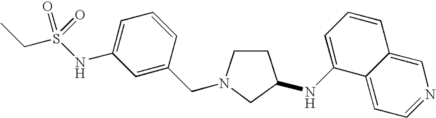 (R)-N-(3-((3-(isoquinolin-5-ylamino)pyrrolidin-1- yl)methyl)phenyl)ethanesulfonamide | 2c, 7, 8, 9, 10, 12a, 15c, 15d |
| 2.042 | 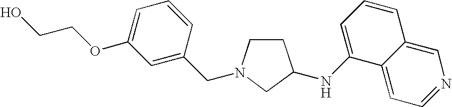 2-(3-((3-(isoquinolin-5-ylamino)pyrrolidin-1- yl)methyl)phenoxy)ethanol | 2c, 7, 8, 9, 10, 12a, 15c, 15d |
| 2.043 | 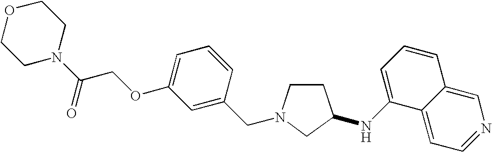 (R)-2-(3-((3-(isoquinolin-5-ylamino)pyrrolidin-1- yl)methyl)phenoxy)-1-morpholinoethanone | 2c, 7, 8, 9, 10, 12a, 15c, 15d |
| 2.044 | 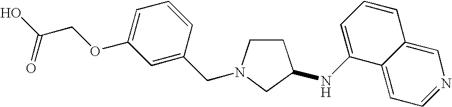 (R)-2-(3-((3-(isoquinolin-5-ylamino)pyrrolidin-1- yl)methyl)phenoxy)acetic acid | 2c, 7, 8, 9, 10, 12a, 15c, 15d |
| 2.045 | 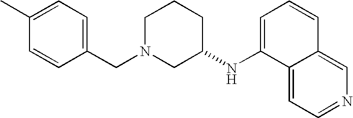 (S)-N-(1-(4-methylbenzyl)piperidin-3-yl)isoquinolin-5- amine | 2c, 7, 8, 9, 10 |
| 2.046 | 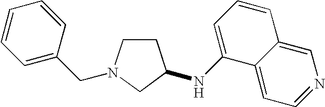 (R)-N-(1-benzylpyrrolidin-3-yl)isoquinolin-5-amine | 2c, 7, 8, 9, 10 |
| 2.047 | 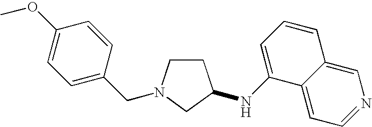 (R)-N-(1-(4-methoxybenzyl)pyrrolidin-3-yl)isoquinolin- 5-amine | 2c, 7, 8, 9, 10 |
| 2.048 | 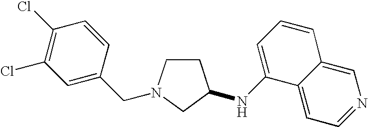 (R)-N-(1-(3,4-dichlorobenzyl)pyrrolidin-3- yl)isoquinolin-5-amine | 2c, 7, 8, 9, 10 |
| 2.049 | 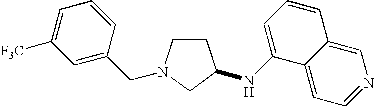 (R)-N-(1-(3-(trifluoromethyl)benzyl)pyrrolidin-3- yl)isoquinolin-5-amine | 2c, 7, 8, 9, 10 |
| 2.050 | 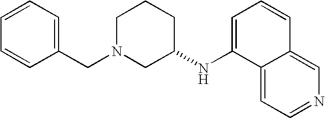 (S)-N-(1-benzylpiperidin-3-yl)isoquinolin-5-amine | 2c, 7, 8, 9, 10 |
| 2.051 | 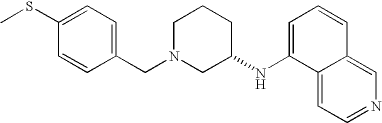 (S)-N-(1-(4-(methylthio)benzyl)piperidin-3- yl)isoquinolin-5-amine | 2c, 7, 8, 9, 10, 12b, 15c, 15e |
| 2.052 | 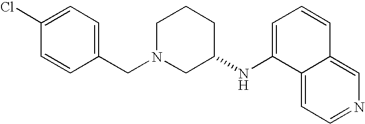 (S)-N-(1-(4-chlorobenzyl)piperidin-3-yl)isoquinolin-5- amine | 2c, 7, 8, 9, 10 |
| 2.053 | 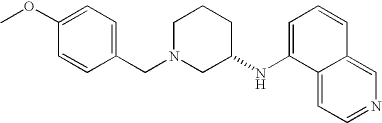 (S)-N-(1-(4-methoxybenzyl)piperidin-3-yl)isoquinolin- 5-amine | 2c, 7, 8, 9, 10 |
| 2.054 | 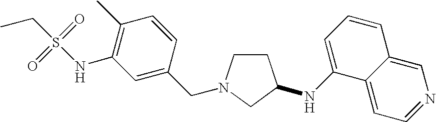 (R)-N-(5-((3-(isoquinolin-5-ylamino)pyrrolidin-1- yl)methyl)-2-methylphenyl)ethanesulfonamide | 2c, 7, 8, 9, 10, 12a, 15c, 15d |
| 2.055 | 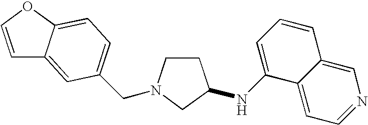 (R)-N-(1-(benzofuran-5-ylmethyl)pyrrolidin-3- yl)isoquinolin-5-amine | 2c, 7, 8, 9, 10 |
| 2.056 | 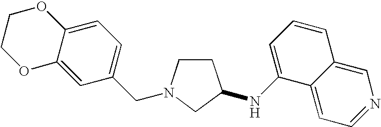 (R)-N-(1-((2,3-dihydrobenzo[b][1,4]dioxin-6- yl)methyl)pyrrolidin-3-yl)isoquinolin-5-amine | 2c, 7, 8, 9, 10 |
| 2.057 | 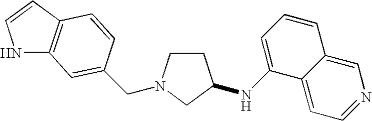 (R)-N-(1-((1H-indol-6-yl)methyl)pyrrolidin-3- yl)isoquinolin-5-amine | 2c, 7, 8, 9, 10 |
| 2.058 | 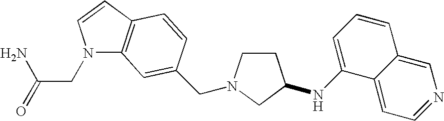 (R)-2-(6-((3-(isoquinolin-5-ylamino)pyrrolidin-1- yl)methyl)-1H-indol-1-yl)acetamide | 2c, 7, 8, 9, 10, 13, 16c |
| 2.059 | 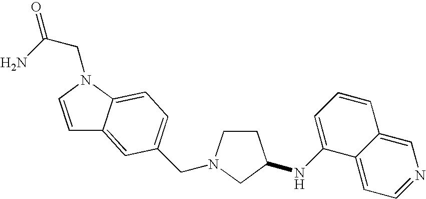 (R)-2-(5-((3-(isoquinolin-5-ylamino)pyrrolidin-1- yl)methyl)-1H-indol-1-yl)acetamide | 2c, 7, 8, 9, 10, 13, 16c |
| 2.060 | 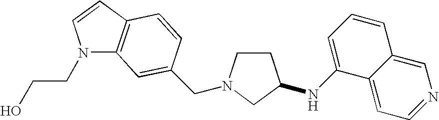 (R)-2-(6-((3-(isoquinolin-5-ylamino)pyrrolidin-1- yl)methyl)-1H-indol-1-yl)ethanol | 2c, 7, 8, 9, 10, 13, 16c |
| 2.061 | 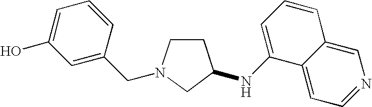 (R)-3-((3-(isoquinolin-5-ylamino)pyrrolidin-1- yl)methyl)phenol | 2c, 7, 8, 9, 10 |
| 2.062 | 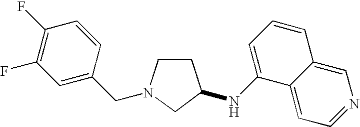 (R)-N-(1-(3,4-difluorobenzyl)pyrrolidin-3-yl)isoquinolin- 5-amine | 2c, 7, 8, 9, 10 |
| 2.063 | 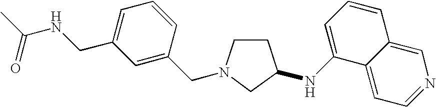 (R)-N-(3-((3-(isoquinolin-5-ylamino)pyrrolidin-1- yl)methyl)benzyl)acetamide | 2c, 7, 8, 9, 10, 13, 16c |
| 2.064 | 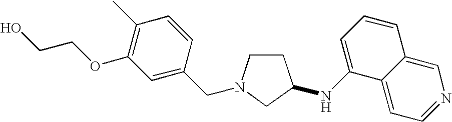 (R)-2-(5-((3-(isoquinolin-5-ylamino)pyrrolidin-1- yl)methyl)-2-methylphenoxy)ethanol | 2c, 7, 8, 9, 10, 12a, 15c, 15d |
| 2.065 | 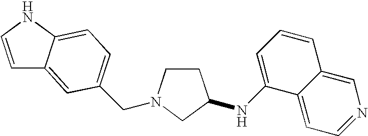 (R)-N-(1-((1H-indol-5-yl)methyl)pyrrolidin-3- yl)isoquinolin-5-amine | 2c, 7, 8, 9, 10 |
| 2.066 | 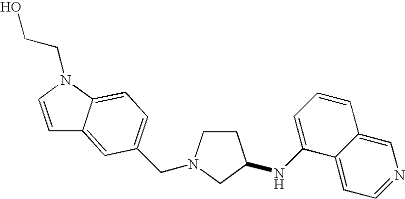 (R)-2-(5-((3-(isoquinolin-5-ylamino)pyrrolidin-1- yl)methyl)-1H-indol-1-yl)ethanol | 2c, 7, 8, 9, 10, 13, 16c |
| 2.067 | 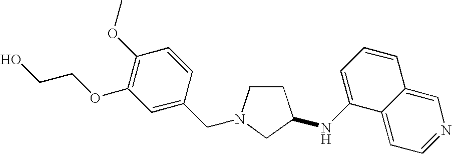 (R)-2-(5-((3-(isoquinolin-5-ylamino)pyrrolidin-1- yl)methyl)-2-methoxyphenoxy)ethanol | 2c, 7, 8, 9, 10, 12a, 15c, 15d |
| 2.068 | 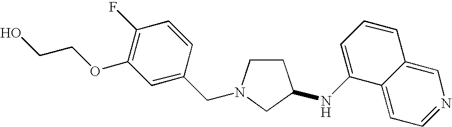 (R)-2-(2-fluoro-5-((3-(isoquinolin-5-ylamino)pyrrolidin- 1-yl)methyl)phenoxy)ethanol | 2c, 7, 8, 9, 10, 12a, 15c, 15d |
| 2.069 | 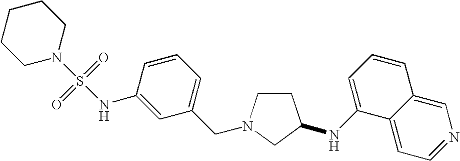 (R)-N-(3-((3-(isoquinolin-5-ylamino)pyrrolidin-1- yl)methyl)phenyl)piperidine-1-sulfonamide | 2c, 7, 8, 9, 10, 12a, 15c, 15d |
| 2.070 | 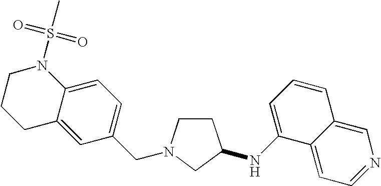 (R)-N-(1-((1-(methylsulfonyl)-1,2,3,4- tetrahydroquinolin-6-yl)methyl)pyrrolidin-3- yl)isoquinolin-5-amine | 2c, 7, 8, 9, 10, 12b, 15c, 15e |
| 2.071 | 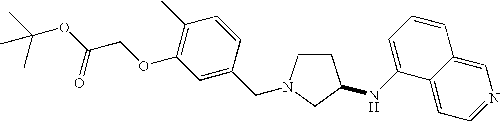 (R)-tert-butyl 2-(5-((3-isoquinolin-5-ylamino)pyrrolidin- 1-yl)methyl)-2-methylphenoxy)acetate | 2c, 7, 8, 9, 10, 12a, 15c, 15d |
| 2.072 | 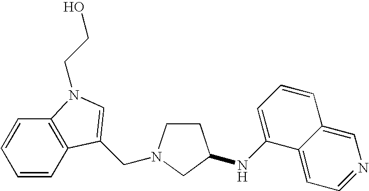 (R)-2-(3-((3-(isoquinolin-5-ylamino)pyrrolidin-1- yl)methyl)-1H-indol-1-yl)ethanol | 2c, 7, 8, 9, 10, 13, 16c |
| 2.073 | 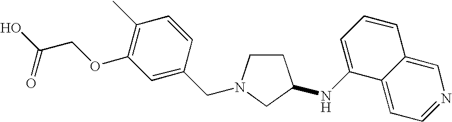 (R)-2-(5-((3-(isoquinolin-5-ylamino)pyrrolidin-1- yl)methyl)-2-methylphenoxy)acetic acid | 2c, 7, 8, 9, 10, 12a, 15c, 15d |
| 2.074 | 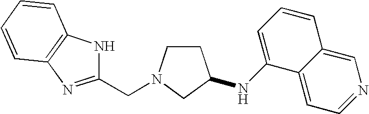 (R)-N-(1-((1H-benzo[d]imidazol-2-yl)methyl)pyrrolidin- 3-yl)isoquinolin-5-amine | 2c, 7, 8, 9, 10 |
| 2.075 | 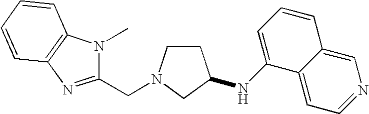 (R)-N-(1-((1-methyl-1H-benzo[d]imidazol-2- yl)methyl)pyrrolidin-3-yl)isoquinolin-5-amine | 2c, 7, 8, 9, 10 |
| 2.076 | 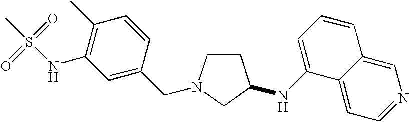 (R)-N-(5-((3-(isoquinolin-5-ylamino)pyrrolidin-1- yl)methyl)-2-methylphenyl)methanesulfonamide | 2c, 7, 8, 9, 10, 12a, 15c, 15d |
| 2.077 | 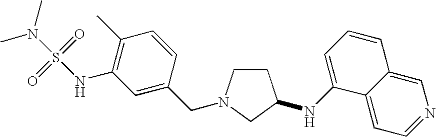 (R)-N-(5-((3-(isoquinolin-5-ylamino)pyrrolidin-1- yl)methyl)-2-methylphenyl)-N′,N′ dimethylaminosulfamide | 2c, 7, 8, 9, 10, 12a, 15c, 15d |
| 2.078 | 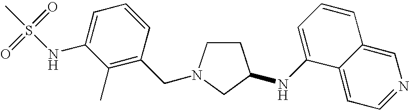 (R)-N-(3-((3-(isoquinolin-5-ylamino)pyrrolidin-1- yl)methyl)-2-methylphenyl)methanesulfonamide | 2c, 7, 8, 9, 10, 12a, 15c, 15d |
| 2.079 | 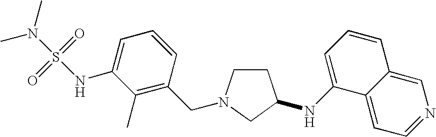 (R)-N-(3-((3-(isoquinolin-5-ylamino)pyrrolidin-1- yl)methyl)-2-methylphenyl)-N′,N′ dimethylaminosulfamide | 2c, 7, 8, 9, 10, 12a, 15c, 15d |
| 2.080 | 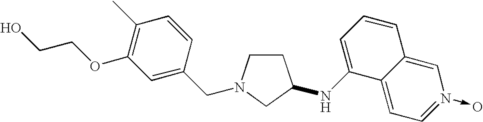 (R)-5-(1-(3-(2-hydroxyethoxy)-4- methylbenzyl)pyrrolidin-3-ylamino)isoquinoline 2-oxide | 2b, 6b, 8, 9, 10, 12a, 15b, 15d |
| 2.081 | 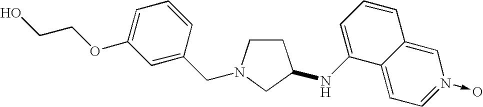 (R)-5-(1-(3-(2-hydroxyethoxy)benzyl)pyrrolidin-3- ylamino)isoquinoline 2-oxide | 2b, 6b, 8, 9, 10, 12a, 15b, 15d |
| 2.082 | 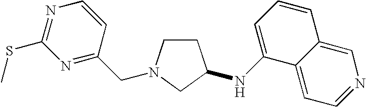 (R)-N-(1-((2-(methylthio)pyrimidin-4- yl)methyl)pyrrolidin-3-yl)isoquinolin-5-amine | 2c, 7, 8, 9, 10, 12b, 15c, 15e |
| 2.083 | 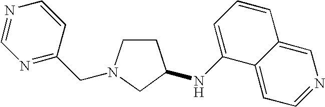 (R)-N-(1-(pyrimidin-4-ylmethyl)pyrrolidin-3- yl)isoquinolin-5-amine | 2c, 7, 8, 9, 10 |
| 2.084 | 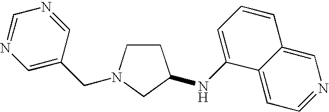 (R)-N-(1-(pyrimidin-5-ylmethyl)pyrrolidin-3- yl)isoquinolin-5-amine | 2c, 7, 8, 9, 10 |
| 2.085 | 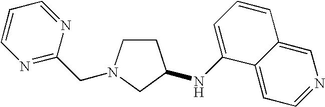 (R)-N-(1-(pyrimidin-2-ylmethyl)pyrrolidin-3- yl)isoquinolin-5-amine | 2c, 7, 8, 9, 10 |
| 2.086 | 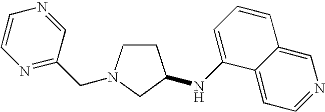 (R)-N-(1-(pyrazin-2-ylmethyl)pyrrolidin-3- yl)isoquinolin-5-amine | 2c, 7, 8, 9, 10 |
| 2.087 | 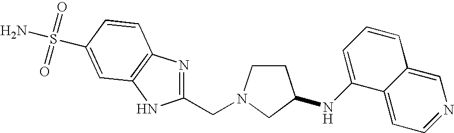 (R)-2-((3-(isoquinolin-5-ylamino)pyrrolidin-1- yl)methyl)-1H-benzo[d]imidazole-6-sulfonamide | 2c, 7, 8, 9, 10, 12b, 15c, 15e |
| 2.088 | 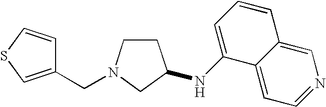 (R)-N-(1-(thiophen-3-ylmethyl)pyrrolidin-3- yl)isoquinolin-5-amine | 2c, 7, 8, 9, 10 |
| 2.089 | 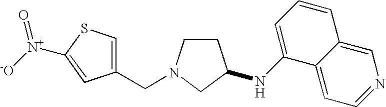 (R)-N-(1-((5-nitrothiophen-3-yl)methyl)pyrrolidin-3- yl)isoquinolin-5-amine | 2c, 7, 8, 9, 10 |
| 2.090 | 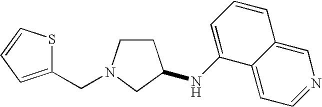 (R)-N-(1-(thiophen-2-ylmethyl)pyrrolidin-3- yl)isoquinolin-5-amine | 2c, 7, 8, 9, 10 |
| 2.091 | 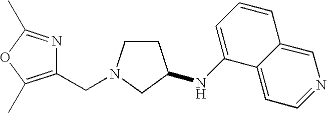 (R)-N-(1-((2,5-dimethyloxazol-4-yl)methyl)pyrrolidin-3- yl)isoquinolin-5-amine | 2c, 7, 8, 9, 10 |
| 2.092 | 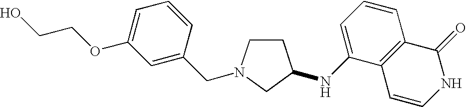 (R)-5-(1-(3-(2-hydroxyethoxy)benzyl)pyrrolidin-3- ylamino)isoquinolin-1(2H)-one | 2b, 6b, 8, 9, 10, 12a, 15b, 15d |
| 2.093 | 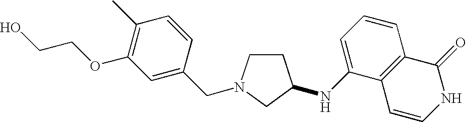 (R)-5-(1-(3-(2-hydroxyethoxy)-4- methylbenzyl)pyrrolidin-3-ylamino)isoquinolin-1(2H)- one | 2b, 6b, 8, 9, 10, 12a, 15b, 15d |
| 2.094 | 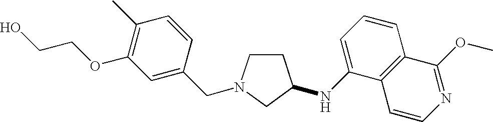 (R)-2-(5-((3-(1-methoxyisoquinolin-5- ylamino)pyrrolidin-1-yl)methyl)-2- methylphenoxy)ethanol | 2b, 6b, 8, 9, 10, 12a, 15b, 15d |
| 2.095 | 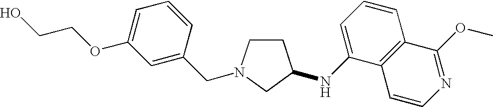 (R)-2-(3-((3-(1-methoxyisoquinolin-5- ylamino)pyrrolidin-1-yl)methyl)phenoxy)ethanol | 2b, 6b, 8, 9, 10, 12a, 15b, 15d |
| 2.096 | 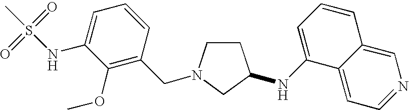 (R)-N-(3-((3-(isoquinolin-5-ylamino)pyrrolidin-1- yl)methyl)-2-methoxyphenyl)methanesulfonamide | 2c, 7, 8, 9, 10, 12a, 15c, 15d |
| 2.097 | 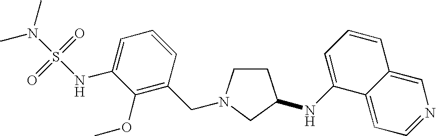 (R)-N-(3-((3-(isoquinolin-5-ylamino)pyrrolidin-1- yl)methyl)-2-methoxyphenyl)-N′,N′ dimethylaminosulfamide | 2c, 7, 8, 9, 10, 12a, 15c, 15d |
| 2.098 | 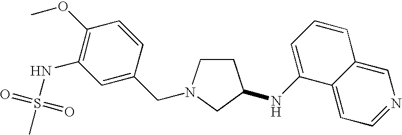 (R)-N-(5-((3-(isoquinolin-5-ylamino)pyrrolidin-1- yl)methyl)-2-methoxyphenyl)methanesulfonamide | 2c, 7, 8, 9, 10, 12a, 15c, 15d |
| 2.099 | 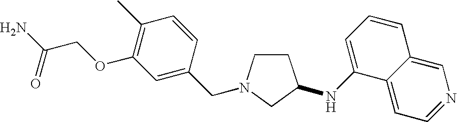 (R)-2-(5-((3-(isoquinolin-5-ylamino)pyrrolidin-1- yl)methyl)-2-methylphenoxy)acetamide | 2c, 7, 8, 9, 10, 12a, 15c, 15d |
| 2.100 | 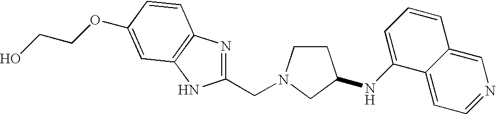 (R)-2-(2-((3-(isoquinolin-5-ylamino)pyrrolidin-1- yl)methyl)-1H-benzo[d]imidazol-6-yloxy)ethanol | 2c, 7, 8, 9, 10, 12a, 15c, 15d |
| 3.001 | 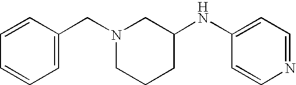 N-(1-benzylpiperidin-3-yl)pyridin-4-amine | 3c, 7, 8, 9, 10 |
| 3.002 | 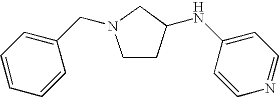 N-(1-benzylpyrrolidin-3-yl)pyridin-4-amine | 3c, 7, 8, 9, 10 |
| 4.001 | 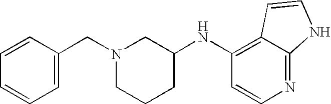 N-(1-benzylpiperidin-3-yl)-1H-pyrrolo[2,3-b]pyridin-4- amine | 4c, 7, 8, 9, 10 |
| 4.002 | 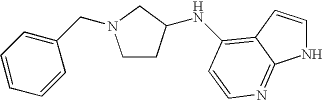 N-(1-benzylpyrrolidin-3-yl)-1H-pyrrolo[2,3-b]pyridin-4- amine | 4c, 7, 8, 9, 10 |
| 5.001 | 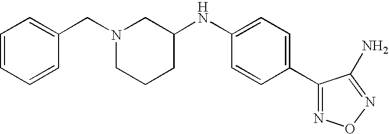 4-(4-(1-benzylpiperidin-3-ylamino)phenyl)-1,2,5- oxadiazol-3-amine | 5a, 7, 8, 9, 10 |
| 5.002 | 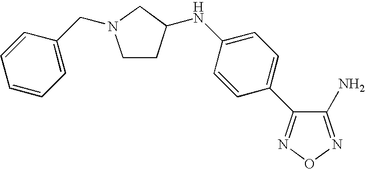 4-(4-(1-benzylpyrrolidin-3-ylamino)phenyl)-1,2,5- oxadiazol-3-amine | 5a, 7, 8, 9, 10 |

Preferred ROCK inhibitor compounds of this invention include, but are not limited to the ROCK inhibitor compounds of embodiments 5, 14, 15, 16, 17, 18, 19, 20, and 21 as described above, and their associated salts, tautomers, solvates, or hydrates. In particular, preferred Compounds include 1.074, 1.075, 1.076, 1.077, 1.079, 1.091, 1.093, 1.108, 1.109, 1.123, 1.124, 1.126, 1.131, 1.132, 1.133, 1.134, 1.135, 1.136, 1.137, 1.138, 1.141, 1.148, 1.149, 1.150, 1.152, 1.153, 1.155, 1.156, 1.157, 1.158, 1.161, 1.162, 1.163, 1.164, 1.165, 1.166, 1.171, 1.173, 1.175, 1.176, 1.186, 1.193, 1.195, 1.197, 1.200, 1.206, 1.212, 1.213, 1.215, 1.217, 1.219, 1.223, 1.233, 1.236, 1.237, 1.238, 1.239, 1.249, 1.252, 1.253, 1.258, 1.259, 1.260, 1.261, 1.262, 1.270, 1.273, 1.275, 1.277, 1.281, 2.025, 2.026, 2.031, 2.038, 2.039, 2.041, 2.046, 2.047, 2.054, 2.055, 2.057, 2.058, 2.059, 2.060, 2.061, 2.064, 2.065, 2.066, 2.067, 2.068, 2.069, 2.072, 2.073, 2.076, 2.077, 2.078, 2.079, 2.082, 2.096, 2.097, and 2.099.

### Pharmaceutical Formulations

The present invention provides a pharmaceutical formulation comprising compounds of Formula I or II and a pharmaceutically acceptable carrier. Pharmaceutically acceptable carriers can be selected by those skilled in the art using conventional criteria. Pharmaceutically acceptable carriers include, but are not limited to, saline solution, aqueous electrolyte solutions, isotonicity modifiers, water polyethers such as polyethylene glycol, polyvinyls such as polyvinyl alcohol and povidone, cellulose derivatives such as methylcellulose and hydroxypropyl methylcellulose, polymers of acrylic acid such as carboxypolymethylene gel, polysaccharides such as dextrans, and glycosaminoglycans such as sodium hyaluronate and salts such as sodium chloride and potassium chloride.

The pharmaceutical formulation useful for the present invention in general is an aqueous solution comprising water, suitable ionic or non-ionic tonicity modifiers, suitable buffering agents, and a compound of Formula I or II. In one embodiment, the compound is at 0.005 to 3% w/v, and the aqueous solution has a tonicity of 200-400 mOsm/kG and a pH of 4-9.

In one embodiment, the tonicity modifier is ionic such as NaCl, for example, in the amount of 0.5-0.9% w/v, preferably 0.6-0.9% w/v.

In another embodiment, the tonicity modifier is non-ionic, such as mannitol, dextrose, in the amount of at least 2%, or at least 2.5%, or at least 3%, and no more than 7.5%; for example, in the range of 3-5%, preferably 4-5% w/v.

The pharmaceutical formulation can be sterilized by filtering the formulation through a sterilizing grade filter, preferably of a 0.22-micron nominal pore size. The pharmaceutical formulation can also be sterilized by terminal sterilization using one or more sterilization techniques including but not limited to a thermal process, such as an autoclaving process, or a radiation sterilization process, or using pulsed light to produce a sterile formulation. In one embodiment, the pharmaceutical formulation is a concentrated solution of the active ingredient; the formulation can be serially diluted using appropriate acceptable sterile diluents prior to administration.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents such as those set forth above, and flavoring agents can be added to provide palatable oral preparations. These compositions can be preserved by the addition of an anti-oxidant such as ascorbic acid.

Pharmaceutical compositions of the invention can be in the form of oil-in-water emulsions. The oily phase can be a vegetable oil, for example olive oil or arachis oil, or a mineral oil, for example liquid paraffin or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monoleate, and condensation products of the said partial esters with ethylene oxide, for example polyoxyethylene sorbitan monoleate. The emulsions can also contain sweetening and flavoring agents.

Pharmaceutical compositions of the invention can be in the form of an aerosol suspension of respirable particles comprising the active compound, which the subject inhales. The respirable particles can be liquid or solid, with a particle size sufficiently small to pass through the mouth and larynx upon inhalation. In general, particles having a size of about 1 to 10 microns, preferably 1-5 microns, are considered respirable.

The pharmaceutical formulation for systemic administration such as injection and infusion is generally prepared in a sterile medium. The active ingredient, depending on the vehicle and concentration used, can either be suspended or dissolved in the vehicle. Adjuvants such as local anesthetics, preservatives and buffering agents can also be dissolved in the vehicle. The sterile injectable preparation can be a sterile injectable solution or suspension in a non-toxic acceptable diluent or solvent. Among the acceptable vehicles and solvents that can be employed are sterile water, saline solution, or Ringer's solution.

The pharmaceutical compositions for oral administration contain active compounds in the form of tablets, lozenges, aqueous or oily suspensions, viscous gels, chewable gums, dispersible powders or granules, emulsion, hard or soft capsules, or syrups or elixirs.

For oral use, an aqueous suspension is prepared by addition of water to dispersible powders and granules with a dispersing or wetting agent, suspending agent one or more preservatives, and other excipients. Suspending agents include, for example, sodium carboxymethylcellulose, methylcellulose and sodium alginate. Dispersing or wetting agents include naturally-occurring phosphatides, condensation products of an allylene oxide with fatty acids, condensation products of ethylene oxide with long chain aliphatic alcohols, condensation products of ethylene oxide with partial esters from fatty acids and a hexitol, and condensation products of ethylene oxide with partial esters derived from fatty acids and hexitol anydrides. Preservatives include, for example, ethyl, and n-propyl p-hydroxybenzoate. Other excipients include sweetening agents (e.g., sucrose, saccharin), flavoring agents and coloring agents. Those skilled in the art will recognize the many specific excipients and wetting agents encompassed by the general description above.

For oral application, tablets are prepared by mixing the active compound with nontoxic pharmaceutically acceptable excipients suitable for the manufacture of tablets. These excipients can be, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets can be uncoated or they can be coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil. Formulation for oral use can also be presented as chewable gums by embedding the active ingredient in gums so that the active ingredient is slowly released upon chewing.

The pharmaceutical compositions can be in the form of suppositories, which are prepared by mixing the active ingredient with a suitable non-irritating excipient that is solid at ordinary temperatures but liquid at the rectal temperature and will thus melt in the rectum to release the compound. Such excipients include cocoa butter and polyethylene glycols.

### Method of Treating Inflammatory Diseases Using Rho Kinase Inhibitor Compounds

The present invention is useful in treating diseases associated with excessive cell proliferation, tissue remodeling, edema and inflammation. The present invention is particularly effective in treating inflammatory disease such as rheumatoid arthritis and inflammatory bowel disease.

### Rheumatoid Arthritis

The inventors have discovered that Rho Kinase inhibitor Compounds of Formula I or II inhibit the ROCK-mediated regulation of chemotaxis, cytokine secretion, edema, or proliferation. The inventors have therefore discovered that Rho Kinase inhibitor Compounds of Formula I or II are useful in treating the defects in inflammation and angiogenesis seen in RA. The present invention is directed to a method of treating RA. The method comprises the steps of first identifying a subject suffering from RA, then administering to the subject an effective amount of a Rho Kinase inhibitor Compound of Formula I or II to treat said disease.

A method for treating RA is based on the properties of a Rho Kinase inhibitor Compound of Formula I or II compounds to reduce at least one of the following processes contributing to pathophysiologies that accompany this disorder: cell proliferation as in angiogenesis, leukocytes chemotaxis and cytokine and chemokine secretion.

Indicia of efficacy for treating rheumatoid arthritis by the present invention include demonstrable improvements in measurable signs, symptoms and other variables relevant to RA. Such improvements include a decrease in swollen and tender joint counts, decrease in pain, improvements in patient and evaluator global assessments of disease activity, decrease in the duration of morning stiffness, decreased levels of fatigue, improvements in appetite and strength, resolution of fever, improved motion of wrist, elbow, neck, shoulder, hip and ankles joints, decreased swollen glands, decreased burning or itching sensation in eyes, inflammation, decreased numbness or tingling, decreased leg ulcers, decreased shortness of breath, improvement of the chronic inflammation of the tendon sheaths, decreased swollen lymph glands, decreased anemia, improved health status, and improved measures of function.

### Inflammatory Bowel Disease

The inventors have discovered that Rho Kinase inhibitor Compounds of Formula I or II inhibit the ROCK-mediated regulation of chemotaxis, cytokine secretion, edema, or proliferation. The inventors have therefore discovered that Rho Kinase inhibitor Compounds of Formula I or II are useful in treating the defects in inflammation, fibrosis, and edema seen in IBD. The present invention is directed to a method of treating IBD. The method comprises the steps of first identifying a subject suffering from IBD, then administering to the subject an effective amount of a Rho Kinase inhibitor Compound of Formula I or II to treat said disease.

A method for treating IBD is based on the properties of Rho Kinase inhibitor Compounds of Formula I or II compounds to reduce at least one of the following processes contributing to pathophysiologies that accompany this disorder: cell proliferation as in fibrosis, leukocyte chemotaxis, cytokine and chemokine secretion, and edema.

Indicia of efficacy for treating inflammatory bowel disease by the present invention include improvement in measurable signs, symptoms and other variables clinically relevant to inflammatory bowel disease. Improvements include: subsiding of an acute episode of disease, maintain non-inflammatory state, weight gain, attenuation of rectal bleeding and pain, decreased urgency or inability to move bowels, decrease in or subsiding of abdominal cramps or pain, alleviation of fatigue and dehydration, prevention of colon rupture and toxic megacolon, firmer stools, decrease in the occurrence of ulcers, reduction of fever, decrease in gastroesophageal reflux, lack of nausea, decrease in chest pain, decrease in abdominal bloating, decrease in gas production, increase in sexual desire, increase in urinary regularity, elimination of mucus from stools, decrease of diarrhea occurrence, decrease in signs of malnutrition, decrease in signs or occurrence of perianal disease, decrease in abdominal mass, decrease in fistulas and strictures, decrease in incidence of related cancers, decrease in inflammation, decrease in edema, decrease in epithelial cell destruction, decrease in fibrosis, decrease in mucous discharge, and decrease in tumor appearance.

An effective amount of a Formula I or II compound is administered to a patient in need of such treatment. The patient either already has the symptoms of at least one above-mentioned disease, or is identified as being at risk of at least one above-mentioned disease. The compound is administered at a frequency that achieves desired efficacy. What constitutes desired efficacy is determined by a physician or other health-care professional. Whether or not sufficient efficacy has been reached is determined by indicia of efficacy for the specific disease. After an initial dose, additional doses are optionally administered if judged to be necessary by a health-care professional.

### Methods of Administration

The present invention is particularly effective in treating inflammatory diseases or conditions such as IA and IBD. Any method of delivering the compound to the target tissues, including local administration and systemic administration, is suitable for the present invention.

In one embodiment, the active compound is delivered by systemic administration; the compound first reaches plasma and then distributes into the target tissues. Examples of systemic administration include oral ingestion, or intravenous or subcutaneous or intraperitoneal or intrathecal or intramuscular administration.

Additional method of systemic administration of the active compound to a subject involves administering a suppository form of the active compound, such that a therapeutically effective amount of the compound reaches the target sites via systemic absorption and circulation.

Another method of systemically administering the active compounds to the subject involves administering a liquid/liquid suspension in the form of eye drops or eye wash or nasal drops of a liquid formulation, or a nasal spray of respirable particles that the subject inhales. Liquid pharmaceutical compositions of the active compound for producing a nasal spray or nasal or eye drops can be prepared by combining the active compound with a suitable vehicle, such as sterile pyrogen free water or sterile saline by techniques known to those skilled in the art.

The active compounds can also be systemically administered to the subject through absorption by the skin using transdermal patches or pads. The active compounds are absorbed into the bloodstream through the skin. Plasma concentration of the active compounds can be controlled by using patches containing different concentrations of active compounds.

For systemic administration, plasma concentrations of active compounds delivered can vary according to compounds; but are generally 1×10⁻¹⁰-1×10⁻⁴ moles/liter, and preferably 1×10⁻⁸-1×10⁻⁵ moles/liter.

Dosage levels about 0.01-140 mg per kg, preferably 0.1-100 mg/kg of body weight per day are useful in the treatment or preventions of conditions involving an inflammatory response (about 0.5 mg to about 7 g per patient per day). Preferred dosage levels are about 0.05-25, or 0.1-10 mg/kg body weight per day. The amount of active ingredient that can be combined with the carrier materials to produce a single dosage form will vary depending upon the host treated and the particular mode of administration. Dosage unit forms will generally contain between from about 1 mg to about 500 mg of an active ingredient.

Injection dose levels range from about 0.1 mg/kg/hour to at least 10 mg/kg/hour, all for from about 1 to about 120 hours and especially 24 to 96 hours. A preloading bolus of from about 0.1 mg/kg to about 10 mg/kg or more can be administered to achieve adequate steady state levels. The maximum total dose in general does not exceed about 2 g/day for a 40 to 80 kg human patient.

Frequency of dosage can also vary depending on the compound used and the particular disease treated. However, for treatment of most disorders, a dosage regimen of p.r.n, 4 times daily, three times daily, or less is preferred, with a dosage regimen of once daily or 2 times daily being particularly preferred.

In another embodiment, the active compound is delivered by inhalation, topical application, or targeted drug delivery to the target tissue. Methods of inhalation include liquid instillation, instillation as a pressurized fluid preparation via metered dose inhaler or equivalent, or inhalation of an aerosolized solution via nebulizer (preferred), inhalation of dry powder (more preferred), and directing soluble or dried material into the air stream during mechanical ventilation (also more preferred).

One administration method is administering to a subject an aerosol suspension of respirable particles comprising the active compound by inhalation. The respirable particles can be liquid or solid, with a particle size sufficiently small to pass through the mouth and larynx upon inhalation; in general, particles ranging from about 1 to 10 microns, but more preferably 1-5 microns, in size are considered respirable. The surface concentrations of active compounds delivered via inhalation can vary according to compounds; but are generally 1×10⁻¹⁰-1×10⁻⁴ moles/liter, and preferably 1×10⁻⁸-1×10⁻⁵ moles/liter.

It is understood, however, that the specific dose level for any particular patient will depend upon a variety of factors including the activity of the specific compound employed, the age, body weight, general health, sex, diet, time of administration, route of administration, and rate of excretion, drug combination (i.e., other drugs being administered to the patient), the severity of the particular disease undergoing therapy, and other factors, including the judgment of the prescribing medical practitioner.

Preferred compounds of the invention will have favorable pharmacological properties. Such properties include, but are not limited to bioavailability, low toxicity, low serum protein binding and desirable in vitro and in vivo half-life.

An example of targeted drug delivery is enclosure of the compound within a liposome, where the liposome is coated with a specific antibody whose antigen is expressed in the targeted lung tissue. It can be advantageous to construe a controlled delivery system of the compounds since such an inhaled product targets the site of action, presents the compound of interest in small regimented quantities and reduces/minimizes any unwanted side effects.

Another example of a delivery system includes microparticulate compositions of the compound. In such a case, the compound is formulated as a microparticulate wherein the carrier is loaded with the compound; such a preparation is then filtered through a fine porous membrane or suitable filtering medium or is exposed to solvent interchanges to produce nanoparticles. Such preparations can be freeze dried or held in suspension in an aqueous or physiologically compatible medium. The preparation so obtained can be inhaled by suitable means.

Another example of a suitable preparation includes a reconstitutable preparation. In this case, the compound is formulated in a preparation to contain the necessary adjuvant to make it physiologically compatible. Such a preparation can be reconstituted by addition of water for injection or suitable physiological fluids, admixed by simple agitation and inhaled using appropriate techniques described above.

The compounds described above can also be prepared into dry powder or equivalent inhalation powders using the well known art of super critical fluid technology. In such a case, the compound is admixed with appropriate excipients and milled into a homogenous mass using suitable solvents or adjuvants. Following this, this mass is subjected to mixing using super critical fluid technology and suitable particle size distribution achieved. The particles in the formulation need to be of a desired particle size range such that the particles can be directly inhaled into the lungs using a suitable inhalation technique or introduced into the lungs via a mechanical ventilator. Alternatively, a formulation can be designed such that the particles are large enough in size thereby offering sufficient surface area to dissolve completely in a suitable fluid when admixed together or to dissolve sufficiently enough prior to nebulization into the lungs.

The invention is illustrated further by the following examples that are not to be construed as limiting the invention in scope to the specific procedures described in them.

### EXAMPLES

### Example 1

### Rho Kinase Inhibition Assay

### Relevance:

This assay demonstrates a compound's ability to inhibit ROCK2 and ROCK1 in an in vitro setting using the isolated enzyme. Compounds having ROCK2 IC₅₀ values on the order of 2 μM or below have been shown to possess efficacy in many studies using in vivo models of the disease processes described in this application.

### Protocol

Inhibition of ROCK2 and ROCK1 activity was determined using the IMAP™ Screening Express Kit (Molecular Devices product number #8073). ROCK2 enzyme (Upstate/Chemicon #14-451), ROCK1 (Upstate/Chemicon #14-601) and Flourescein tagged substrate peptide Fl-AKRRRLSSLRA (Molecular Devices product number R7184) was pre-incubated with a test compound (a Formula II compound or other rho kinase compound such as fasudil, H-1152, H7, Y-27632, Y-39983) for 5 minutes in buffer containing 10 mM Tris-HCl pH 7.2, 10 mM MgCl₂, and 0.1% BSA. Following the pre-incubation, 10 μM ATP was added to initiate the reaction. After 60 minutes at room temperature, Molecular Devices IMAP™ binding solution was added to bind phosphorylated substrate. After 30 minutes of incubation in the presence of the IMAP™ beads, the fluorescence polarization was read and the ratio was reported as mP. IC₅₀ values for compounds and EC₅₀ values for ATP were calculated using the Prism software from Graphpad.

### Results:

TABLE 1
Rho Kinase I and II Potency Data

| | ROCK1 Ki, | ROCK1 Ki, | ROCK2 Ki, | ROCK2 Ki, |
|---|---|---|---|---|
| Compound | Avg, nM | StdDev, nM | Avg, nM | StdDev, nM |
| 1.008 | 30.5 | 0.8 | 3.9 | 0.1 |
| 1.034 | 36.0 | 22.2 | 5.3 | 2.6 |
| 1.039 | 208.6 | 109.0 | 24.7 | 8.4 |
| 1.051 | 37.2 | 4.0 | 3.8 | 0.0 |
| 1.072 | 33.7 | 22.1 | 5.6 | 3.1 |
| 1.074 | 40.1 | 3.3 | 4.1 | 1.5 |
| 1.075 | 48.7 | 2.8 | 4.4 | 0.3 |
| 1.076 | 14.3 | 5.4 | 2.6 | 0.6 |
| 1.077 | 76.1 | 30.9 | 11.1 | 5.8 |
| 1.078 | 36.3 | 10.1 | 3.6 | 0.9 |
| 1.079 | 71.5 | 9.1 | 4.7 | 1.1 |
| 1.080 | 130.8 | 42.6 | 15.2 | 4.4 |
| 1.087 | 84.1 | 11.1 | 15.4 | 1.4 |
| 1.090 | 281.0 | 103.7 | 24.9 | 7.9 |
| 1.091 | 71.4 | 22.0 | 3.3 | 1.0 |
| 1.092 | 190.5 | 42.2 | 28.4 | 10.6 |
| 1.093 | 64.5 | 21.9 | 7.7 | 5.2 |
| 1.095 | 274.8 | 88.0 | 49.5 | 35.9 |
| 1.098 | 205.6 | 69.4 | 25.0 | 6.4 |
| 1.106 | 223.4 | 82.0 | 15.1 | 4.9 |
| 1.107 | 233.7 | 137.2 | 14.0 | 8.5 |
| 1.108 | 25.6 | 3.2 | 6.5 | 0.3 |
| 1.109 | 58.8 | 25.8 | 9.6 | 2.5 |
| 1.110 | 59.0 | 4.1 | 11.2 | 0.3 |
| 1.115 | 89.7 | 17.5 | 20.6 | 1.7 |
| 1.116 | 257.8 | 45.6 | 48.9 | 5.5 |
| 1.117 | 208.0 | 1.9 | 35.8 | 2.3 |
| 1.118 | 461.7 | 28.3 | 81.7 | 52.7 |
| 1.123 | 82.3 | 11.0 | 9.6 | 4.3 |
| 1.124 | 64.5 | 7.9 | 3.3 | 0.8 |
| 1.125 | 557.1 | 1.7 | 50.9 | 16.8 |
| 1.126 | 76.2 | 16.7 | 17.2 | 3.9 |
| 1.127 | 96.6 | 11.6 | 11.2 | 0.4 |
| 1.130 | 577.1 | 340.0 | 142.0 | 38.1 |
| 1.131 | 19.7 | 5.9 | 3.8 | 0.9 |
| 1.132 | 22.5 | 6.5 | 3.5 | 0.4 |
| 1.133 | 25.0 | 7.2 | 4.3 | 1.1 |
| 1.134 | 22.4 | 6.0 | 4.4 | 0.6 |
| 1.136 | 40.3 | 15.3 | 5.4 | 0.4 |
| 1.137 | 25.8 | 10.7 | 5.1 | 1.2 |
| 1.138 | 36.3 | 12.2 | 7.2 | 1.1 |
| 1.139 | 200.3 | 26.3 | 23.2 | 9.6 |
| 1.140 | 236.1 | 199.3 | 32.9 | 24.9 |
| 1.141 | 28.5 | 11.1 | 3.8 | 1.1 |
| 1.142 | 104.2 | 26.6 | 12.0 | 4.4 |
| 1.143 | 49.7 | 30.8 | 12.6 | 11.9 |
| 1.144 | 97.6 | 65.0 | 19.5 | 13.0 |
| 1.145 | 35.0 | 13.5 | 6.4 | 0.9 |
| 1.146 | 39.8 | 10.9 | 10.7 | 1.5 |
| 1.147 | 58.3 | 15.6 | 45.7 | 52.0 |
| 1.148 | 24.3 | 13.7 | 3.6 | 0.9 |
| 1.149 | 46.8 | 21.3 | 4.2 | 2.2 |
| 1.150 | 33.2 | 17.5 | 3.2 | 1.2 |
| 1.151 | 22.8 | 6.0 | 2.9 | 0.5 |
| 1.152 | 19.8 | 13.3 | 3.3 | 0.9 |
| 1.153 | 62.8 | 8.7 | 4.2 | 0.8 |
| 1.154 | 52.7 | 9.5 | 6.6 | 1.0 |
| 1.155 | 45.4 | 14.7 | 7.0 | 2.0 |
| 1.156 | 135.8 | 34.3 | 13.0 | 3.0 |
| 1.157 | 263.8 | 73.9 | 8.8 | 1.6 |
| 1.158 | 64.1 | 20.1 | 5.1 | 1.0 |
| 1.159 | 48.1 | 9.2 | 10.1 | 2.6 |
| 1.160 | 218.3 | 28.3 | 49.4 | 13.4 |
| 1.161 | 9.9 | 3.4 | 2.5 | 0.5 |
| 1.162 | 15.2 | 1.5 | 2.8 | 0.8 |
| 1.163 | 33.6 | 5.8 | 2.9 | 0.4 |
| 1.164 | 42.4 | 7.2 | 6.1 | 1.2 |
| 1.165 | 50.7 | 4.4 | 3.4 | 0.6 |
| 1.166 | 95.2 | 8.6 | 8.0 | 0.8 |
| 1.167 | 118.6 | 17.1 | 18.5 | 1.7 |
| 1.168 | 162.2 | 68.3 | 22.9 | 10.4 |
| 1.169 | 256.2 | 132.7 | 33.8 | 20.0 |
| 1.170 | 80.0 | 25.9 | 12.5 | 6.1 |
| 1.171 | 109.2 | 60.1 | 16.0 | 8.4 |
| 1.172 | 103.0 | 40.6 | 20.5 | 7.3 |
| 1.173 | 15.1 | 6.8 | 3.6 | 1.0 |
| 1.175 | 65.9 | 28.3 | 7.6 | 1.5 |
| 1.176 | 314.3 | 77.6 | 11.2 | 3.2 |
| 1.177 | 156.1 | 55.0 | 18.2 | 5.5 |
| 1.178 | 137.6 | 58.0 | 24.9 | 17.6 |
| 1.179 | 292.0 | 70.7 | 19.3 | 4.4 |
| 1.180 | 138.5 | 46.5 | 23.1 | 4.8 |
| 1.181 | 567.8 | 191.3 | 32.8 | 3.5 |
| 1.182 | 408.3 | 106.6 | 30.6 | 4.3 |
| 1.183 | 165.1 | 46.3 | 16.8 | 3.7 |
| 1.184 | 843.1 | 53.0 | 90.9 | 13.9 |
| 1.185 | 81.6 | 33.0 | 12.6 | 6.4 |
| 1.186 | 129.3 | 42.2 | 11.9 | 4.9 |
| 1.187 | 296.2 | 78.8 | 17.3 | 5.8 |
| 1.188 | 3468.8 | | 652.7 | |
| 1.189 | 187.9 | 62.0 | 34.3 | 5.1 |
| 1.190 | 325.6 | 38.9 | 71.8 | 9.0 |
| 1.191 | 147.3 | 24.7 | 33.4 | 2.0 |
| 1.192 | 158.4 | 33.5 | 37.7 | 4.7 |
| 1.193 | 64.9 | 4.2 | 14.8 | 1.2 |
| 1.194 | 175.7 | 6.3 | 20.2 | 2.4 |
| 1.195 | 196.2 | 58.0 | 10.3 | 3.6 |
| 1.196 | 710.7 | 191.7 | 39.8 | 15.0 |
| 1.197 | 120.2 | 36.0 | 5.0 | 1.4 |
| 1.198 | 584.5 | 139.5 | 24.7 | 9.9 |
| 1.199 | 1856.6 | | 213.0 | 34.4 |
| 1.200 | 76.5 | 17.9 | 5.9 | 0.9 |
| 1.201 | 1585.4 | | 229.5 | |
| 1.202 | 203.5 | 40.9 | 33.0 | 2.1 |
| 1.203 | 329.4 | 67.4 | 41.6 | 6.4 |
| 1.204 | 196.1 | 42.0 | 31.9 | 2.2 |
| 1.205 | 498.1 | 95.2 | 46.4 | 3.7 |
| 1.206 | 64.4 | 15.1 | 9.1 | 3.8 |
| 1.207 | 516.3 | 27.5 | 43.7 | 1.1 |
| 1.208 | 54.2 | 25.0 | 12.9 | 2.8 |
| 1.209 | 4591.0 | | 469.6 | 58.3 |
| 1.210 | 95.1 | 18.2 | 25.5 | 3.8 |
| 1.211 | 395.5 | 58.5 | 57.6 | 0.6 |
| 1.212 | 44.2 | 11.2 | 3.9 | 0.2 |
| 1.213 | 106.3 | 10.9 | 3.0 | 0.5 |
| 1.214 | 546.5 | 10.9 | 143.0 | 7.0 |
| 1.215 | 102.8 | 5.8 | 3.5 | 0.3 |
| 1.216 | 1885.4 | | 402.9 | 79.5 |
| 1.217 | 70.1 | 9.5 | 12.1 | 1.1 |
| 1.218 | 401.8 | 34.4 | 30.7 | 3.0 |
| 1.219 | 343.6 | 37.6 | 15.4 | 2.3 |
| 1.221 | 264.4 | 41.6 | 30.0 | 2.6 |
| 1.222 | 228.8 | 41.9 | 75.5 | 1.2 |
| 1.223 | 239.5 | 21.5 | 15.7 | 1.9 |
| 1.224 | 487.0 | 151.5 | 77.5 | 23.0 |
| 1.225 | 605.0 | 133.2 | 189.4 | 48.9 |
| 1.226 | 91.7 | 31.5 | 8.8 | 2.6 |
| 1.227 | 47.5 | 2.8 | 5.3 | 0.4 |
| 1.228 | 1883.4 | 681.9 | 139.6 | 28.2 |
| 1.229 | 121.4 | 86.2 | 18.4 | 5.8 |
| 1.230 | 345.9 | 85.2 | 35.3 | 9.8 |
| 1.231 | 305.1 | 62.8 | 60.3 | 18.2 |
| 1.232 | 136.6 | 41.1 | 20.8 | 8.8 |
| 1.233 | 47.2 | 7.2 | 1.3 | 0.1 |
| 1.234 | 1735.2 | 179.0 | 166.4 | 11.6 |
| 1.235 | 1386.4 | 173.1 | 335.4 | 29.4 |
| 1.236 | 49.3 | 7.1 | 2.1 | 0.1 |
| 1.237 | 286.7 | 55.0 | 4.0 | 0.4 |
| 1.238 | 61.2 | 22.1 | 1.5 | 0.3 |
| 1.239 | 282.6 | 36.2 | 6.3 | 0.6 |
| 1.240 | 624.8 | 74.2 | 60.1 | 9.3 |
| 1.241 | 65.1 | 11.8 | 21.0 | 6.4 |
| 1.242 | 71.4 | 14.1 | 17.5 | 1.8 |
| 1.243 | 219.3 | 29.7 | 84.3 | 17.2 |
| 1.244 | 683.1 | 80.9 | 138.7 | 25.4 |
| 1.245 | 199.0 | 27.7 | 49.5 | 7.9 |
| 1.246 | 92.1 | 6.3 | 11.2 | 0.8 |
| 1.247 | 1312.4 | 268.7 | 242.6 | 53.1 |
| 1.248 | 2349.7 | | 890.6 | 509.8 |
| 1.249 | 91.7 | 25.0 | 8.6 | 3.8 |
| 1.250 | 247.0 | 63.7 | 45.8 | 13.8 |
| 1.251 | 206.8 | 44.0 | 49.2 | 10.5 |
| 1.252 | 30.5 | 1.5 | 4.5 | 0.4 |
| 1.253 | 59.9 | 7.4 | 1.7 | 0.2 |
| 1.254 | 116.0 | 19.4 | 39.0 | 8.7 |
| 1.255 | 3559.3 | 1202.9 | 358.9 | 99.3 |
| 1.256 | 700.1 | 179.5 | 85.5 | 18.8 |
| 1.257 | 1273.7 | 237.3 | 168.0 | 35.4 |
| 1.258 | 9.5 | 3.5 | 1.3 | 0.4 |
| 1.259 | 19.5 | 11.6 | 2.1 | 0.3 |
| 1.260 | 70.9 | 48.0 | 7.1 | 1.9 |
| 1.261 | 307.4 | 139.0 | 14.8 | 6.5 |
| 1.262 | 54.9 | 13.3 | 4.0 | 0.7 |
| 1.263 | 2130.5 | 673.5 | 453.4 | 105.3 |
| 1.264 | 494.5 | 1.1 | 59.4 | 9.5 |
| 1.265 | 161.7 | 25.9 | 21.6 | 0.8 |
| 1.266 | 53.8 | 15.1 | 17.1 | 2.8 |
| 1.267 | 98.8 | 21.6 | 23.9 | 6.2 |
| 1.268 | 403.6 | 78.8 | 40.7 | 7.5 |
| 1.269 | 239.1 | 62.6 | 22.8 | 9.0 |
| 1.270 | 130.5 | 45.0 | 9.9 | 0.6 |
| 1.271 | 332.1 | 99.9 | 77.7 | 5.8 |
| 1.272 | 1823.7 | 1294.6 | 194.3 | 17.0 |
| 1.273 | 31.3 | 8.3 | 8.2 | 1.0 |
| 1.274 | 223.4 | 46.3 | 10.7 | 1.1 |
| 1.275 | 401.7 | 44.9 | 14.1 | 2.0 |
| 1.276 | 64.2 | 5.2 | 12.3 | 2.5 |
| 1.277 | 42.3 | 10.4 | 4.6 | 1.3 |
| 1.278 | 80.2 | 10.5 | 10.2 | 1.8 |
| 1.279 | 455.9 | 20.3 | 34.2 | 1.6 |
| 1.280 | 746.0 | 58.3 | 38.0 | 4.0 |
| 1.281 | 71.8 | | 7.4 | |
| 2.007 | 390.4 | | 179.1 | |
| 2.016 | 100.5 | 14.8 | 42.4 | 10.2 |
| 2.020 | 100.5 | 13.1 | 36.5 | 4.7 |
| 2.022 | 44.8 | 6.9 | 15.3 | 1.1 |
| 2.025 | 6.9 | 1.3 | 2.9 | 0.5 |
| 2.026 | 38.0 | 15.2 | 13.0 | 4.1 |
| 2.027 | 15.7 | 3.8 | 7.4 | 2.3 |
| 2.031 | 14.6 | 4.9 | 5.3 | 1.2 |
| 2.034 | 1002.6 | 392.4 | 221.1 | 312.7 |
| 2.035 | 601.0 | | 201.9 | |
| 2.036 | 579.5 | 139.9 | 232.8 | |
| 2.037 | 920.8 | | 182.2 | |
| 2.038 | 28.9 | 4.5 | 6.3 | 1.0 |
| 2.039 | 18.8 | 9.6 | 6.7 | 1.9 |
| 2.040 | 59.6 | 10.7 | 25.4 | 5.0 |
| 2.041 | 30.8 | 2.6 | 9.6 | 2.6 |
| 2.043 | 49.4 | 9.5 | 21.5 | 2.4 |
| 2.044 | 81.4 | 20.2 | 24.1 | 3.7 |
| 2.045 | 90.6 | 64.6 | 88.0 | 57.3 |
| 2.046 | 16.7 | 1.1 | 5.6 | 0.8 |
| 2.047 | 26.4 | 3.6 | 7.0 | 2.3 |
| 2.048 | 71.5 | 22.8 | 34.6 | 9.7 |
| 2.049 | 113.0 | 42.1 | 48.0 | 17.1 |
| 2.050 | 367.7 | 115.4 | 250.7 | |
| 2.051 | 1437.2 | 595.4 | 1179.8 | |
| 2.052 | 508.5 | 169.1 | 142.6 | |
| 2.053 | 951.6 | 157.1 | 182.4 | |
| 2.054 | 17.1 | 2.3 | 3.7 | 0.1 |
| 2.055 | 16.0 | 5.3 | 6.4 | 1.2 |
| 2.056 | 106.6 | 12.7 | 48.7 | 26.5 |
| 2.057 | 6.2 | 1.3 | 3.7 | 0.7 |
| 2.058 | 15.3 | 2.8 | 3.3 | 0.6 |
| 2.059 | 3.9 | 0.3 | 2.7 | 0.2 |
| 2.060 | 4.9 | 0.3 | 3.2 | 0.1 |
| 2.061 | 10.5 | 3.2 | 1.8 | 0.4 |
| 2.062 | 63.4 | 25.1 | 30.5 | 2.2 |
| 2.063 | 206.2 | 88.8 | 73.9 | 3.5 |
| 2.064 | 4.1 | 1.8 | 2.2 | 0.4 |
| 2.065 | 4.1 | 1.4 | 1.8 | 0.2 |
| 2.066 | 10.2 | 3.4 | 2.3 | 0.4 |
| 2.067 | 19.6 | 5.8 | 4.2 | 0.5 |
| 2.068 | 8.0 | 2.0 | 5.8 | 0.4 |
| 2.069 | 16.7 | 4.9 | 2.4 | 0.3 |
| 2.070 | 285.9 | 122.0 | 48.4 | 6.1 |
| 2.071 | 21.2 | 2.7 | 11.9 | 0.5 |
| 2.072 | 7.5 | 1.4 | 4.4 | 0.5 |
| 2.073 | 12.7 | 2.6 | 4.2 | 0.4 |
| 2.074 | 133.3 | 31.1 | 36.4 | 7.7 |
| 2.075 | 123.0 | 25.7 | 21.7 | 1.5 |
| 2.076 | 8.0 | 1.8 | 2.4 | 0.3 |
| 2.077 | 33.7 | 12.5 | 5.0 | 0.8 |
| 2.078 | 18.3 | 4.4 | 2.6 | 0.0 |
| 2.079 | 18.5 | 5.5 | 2.3 | 0.2 |
| 2.080 | 213.7 | 18.5 | 125.9 | 17.7 |
| 2.081 | 1446.1 | 317.4 | 1111.2 | 989.8 |
| 2.082 | 131.7 | 30.1 | 9.0 | 2.9 |
| 2.083 | 1882.9 | 380.5 | 857.6 | 706.9 |
| 2.084 | 1174.6 | 172.9 | 349.6 | 116.2 |
| 2.085 | 2391.7 | 219.6 | 812.0 | 417.7 |
| 2.086 | 1246.0 | 57.7 | 358.0 | 28.5 |
| 2.087 | 896.4 | 67.0 | 59.3 | 6.2 |
| 2.088 | 38.7 | 6.1 | 13.6 | 1.6 |
| 2.089 | 102.1 | 3.7 | 32.9 | 3.1 |
| 2.090 | 53.3 | 10.2 | 19.5 | 2.4 |
| 2.091 | 776.1 | 94.2 | 236.7 | 16.1 |
| 2.092 | 1132.5 | 128.2 | 458.0 | 73.1 |
| 2.093 | 576.3 | 99.5 | 127.7 | 19.5 |
| 2.094 | 16570.6 | 1465.6 | | |
| 2.096 | 70.2 | 9.7 | 9.6 | 1.5 |
| 2.097 | 35.4 | 2.1 | 2.8 | 0.8 |
| 2.098 | 382.5 | 13.6 | 73.5 | 3.6 |
| 2.099 | 15.0 | | 3.8 | |
| fasudil | 346.3 | 17.6 | 96.4 | 6.4 |
| H-1152 | 18.5 | 5.3 | 2.0 | 0.3 |
| H7 | | | 124.7 | 5.6 |
| Y-27632 | 197.2 | 50.6 | 60.9 | 16.9 |
| Y-39983 | 34.7 | 11.1 | 3.6 | 0.9 |

### Conclusion

Most of the compounds studied inhibited ROCK2 with a Kᵢ below 600 nM, many of these values below 60 nM. The most potent compounds in this assay showed Kᵢ values below 15 nM.

### Example 2

### IL-1β Monocyte Secretion Assay

### Relevance

This assay is an in vitro assay of cytokine secretion that can be used to evaluate the ability of Rho Kinase inhibitor compounds of Formula I or II to inhibit cytokine secretion, as the secretion of cytokines contributes to the inflammation in both RA and IBD.

### Protocol

Peripheral blood from healthy human volunteers was collected and the monocytes isolated via Ficoll-paque density centrifugation. The resultant pellet was re-suspended in media containing 1 ng/mL lipopolysaccharide (LPS) and plated at a density of 500,000 cells/mL. After 3 hours of incubation (37° C., 5% CO₂, humidified air), monocytes were selected by adherence to the tissue culture plastic by washing wells with media. Following the media wash, cells were incubated for 2 minutes with the Rho Kinase inhibitors (10 μM) prior to the addition of 1 mM ATP. Cells were allowed to incubate with compounds for 30 minutes at 37° C. after which the supernatant was removed for immediate determination of IL-1β concentration. The concentration of IL-1β in cell supernatants was measured using the Human IL-1β kit and Bio-Plex system (Bio-Rad) according to manufacture's instructions.

### Results

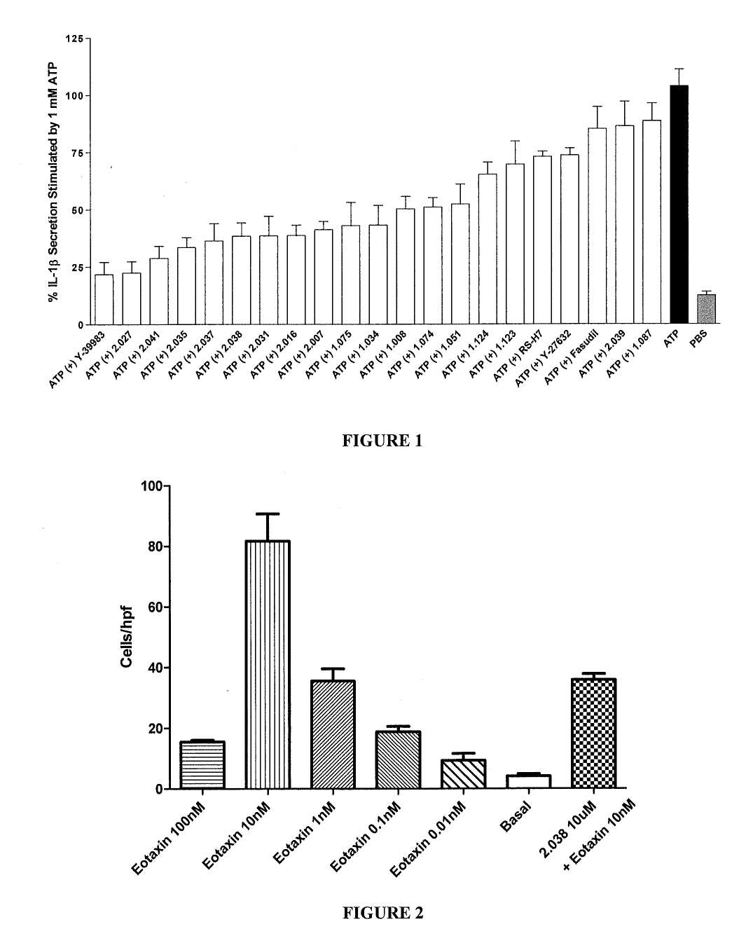

FIG. 1 shows percent inhibition of IL-1β secretion in human monocytes by Rho Kinase inhibitors Compounds of Formula I or II. The tested Rho Kinase inhibitors Compounds of Formula I or II at a 10 μM concentration demonstrated a varying efficacy range. Many compounds effectively reduced IL-1β secretion to low levels. A few compounds showed little effect on decreasing the ATP stimulated release of IL-1β.

### Example 3

### Human Neutrophil Chemotaxis

### Relevance

This assay is an in vitro assay of neutrophil chemotaxis that can be used to evaluate the ability of Rho Kinase inhibitor compounds of Formula I or II to inhibit the migration of human neutrophils, an inflammatory cell that has been implicated in the pathophysiology of both RA and IBD.

### Protocol

Peripheral blood from healthy human volunteers was collected and the neutrophils were isolated by Ficoll-paque density centrifugation followed by dextran sedimentation and hypotonic lysis of the red blood cells. Neutrophil chemotaxis was assessed using a modified Boyden Chamber (Neuroprobe, 96-well) with a 3 μm pore polycarbonate membrane. The ability of the tested compounds to block chemotaxis induced by a 1 μM fMLP challenge during a one hour incubation at 37° C. with 5% CO₂ was assessed in a dose response manner. The results are shown in Table 2.

### Results

The results demonstrate that Rho Kinase inhibition by Formula I or II compounds inhibited human neutrophil migration toward a chemotactic stimulant in vitro with IC₅₀ potencies ranging from less than 1 μM to nearly 24 μM (Table 2)

TABLE 2
Inhibition of fMLP-induced neutrophil chemotaxis by Rho
kinase inhibitors compounds of Formula I and/or II.

| | Chemotaxis | |
|---|---|---|
| Compound | Avg. IC₅₀ | Chemotaxis |
| Number | (nM) | SEM (nM) |
| 2.038 | 734 | 367 |
| Y-39983 | 1,390 | 803 |
| 1.131 | 1,587 | 916 |
| 2.039 | 1,643 | 949 |
| 2.025 | 1,650 | 636 |
| 1.138 | 1,850 | 212 |
| 1.091 | 2,332 | 2,077 |
| 1.136 | 2,600 | 424 |
| 1.092 | 2,747 | 1,586 |
| 2.036 | 2,767 | 1,597 |
| 1.123 | 3,050 | 778 |
| 1.124 | 3,402 | 1,964 |
| 2.026 | 3,800 | 2,970 |
| H-1152 | 4,350 | 1,202 |
| 1.087 | 4,500 | 2,598 |
| 2.034 | 4,733 | 2,733 |
| 1.034 | 5,601 | 3,234 |
| 2.035 | 6,600 | 3,811 |
| Y-27632 | 6,765 | 1,747 |
| Fasudil | 23,800 | 13,741 |

### Example 4

### Human and Murine Eosinophil Chemotaxis

### Relevance

These assays are in vitro assays of eosinophil chemotaxis that can be used to evaluate the ability of Rho Kinase inhibitor compounds of Formula I or II to inhibit the migration of eosinophils, an inflammatory cell involved in the pathophysiology of RA.

### Protocol

Human Eosinophil Isolation: Peripheral blood from healthy human volunteers was collected and the PMNs separated via Ficoll-paque density centrifugation followed by hypotonic lysis of the red blood cells. Subsequently, the human eosinophils were isolated from the cell suspension via StemCell Technologies Human Eosinophil Enrichment kit (Cat. No 19256) according to the manufacturer's recommendations. Briefly, unwanted cells were specifically labeled with dextran-coated magnetic nanoparticles using bispecific Tetrameric Antibody Complexes (TAC) directed against cell surface antigens on human blood cells: CD2, CD3, CD14, CD16, CD19, CD20CD36, CD56, CD123, glycophorin A and dextran. The unwanted cells are then separated from the unlabelled eosinophils using the EasySep® magnetic isolation procedure.

Mouse Eosinophil Isolation: Bronchoalveolar lavage was collected from ovalbumin sensitized and challenged mice in a volume of 2.5 mL lavage buffer. The lavage buffer was 0.9% saline with 10% fetal bovine serum. The pooled lavages were maintained on ice until use. The murine eosinophils were isolated using MACS cell separation (Miltenyi Biotech) by depletion of B cells and T cells by positive selection following incubation with antibody conjugated magnetic beads specific for CD45-R (B220) and CD90 (Thy 1.2), which bind B cells and T cells, respectively.

In Vitro Chemotaxis: Eosinophil chemotaxis was assessed using a modified Boyden Chamber (Neuroprobe, 96-well) with a 5 μm pore membrane. The ability of the tested compounds to block chemotaxis induced by a 10 nM eotaxin challenge (mouse) or 1 nM eotaxin challenge (human) during one hour incubation at 37° C. with 5% CO₂ was assessed.

### Results

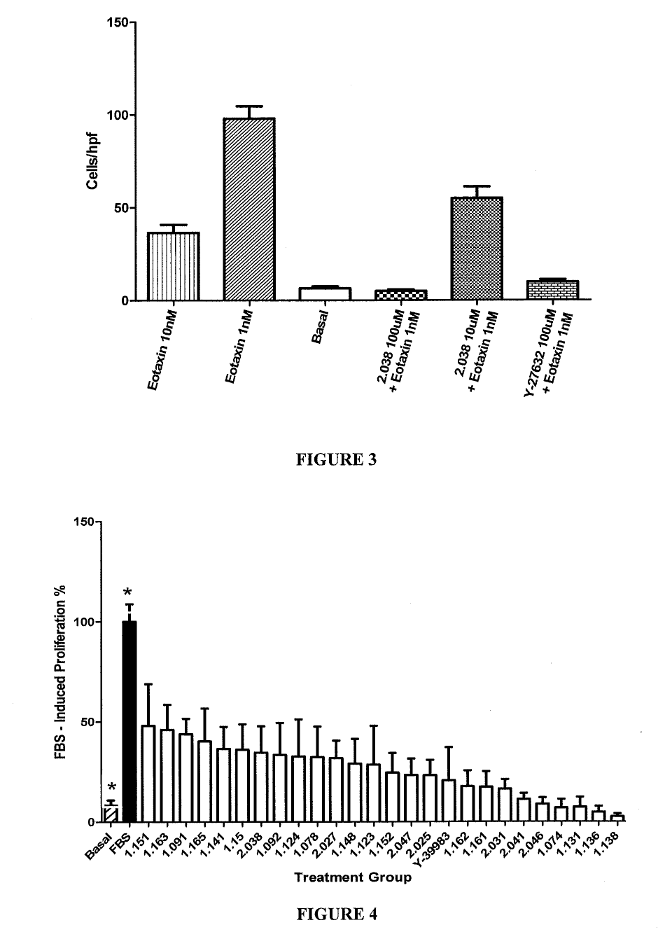

Chemotaxis was quantified via microscopy by counting the number of migrated cells in at least 3 view fields per treatment. The results are shown in FIGS. 2 and 3. FIG. 2 demonstrates that chemotaxis was induced by eotaxin in murine eosinophils; the chemotactic response was subsequently inhibited by Rho Kinase inhibitor Compound 2.038. FIG. 3 demonstrates that chemotaxis was induced by eotaxin in human eosinophils. The chemotactic response was subsequently inhibited by Rho Kinase inhibitor Compound 2.038.

### Example 5

### Smooth Muscle Proliferation Assay

### Relevance

Cellular proliferation is an important process in remodeling effects such as angiogenesis and fibrosis. This assay measures the ability of compounds of this invention to regulate proliferation.

### Protocol

Effects on cell proliferation were measured using a radiographic technique know as [³H] thymidine incorporation. A-10 rat thoracic aorta cells (ATCC #CRL 1476) were grown on 24-well plates in Dulbecco's Modified Eagles Medium-High Glucose (Gibco cat. # 11995-065) containing 10% Fetal Bovine Serum (Sigma EC# 232-690-6) for 24 hrs in an incubator at 37° C. Growth media was then removed, the cells were washed with warmed PBS (Gibco cat# 14190-144) and warmed serum free media containing 0.1% BSA in order to force the cells into a quiescent state. 24 hours later the media was removed and replaced with warmed serum free media containing from 10 nM to 30 uM of test compound. The cells were incubated for 60 min at 37° C. The cells were then stimulated with either 10% FBS or 10 ng/mL PDGF (BD Biosciences cat# 354051) and placed in an incubator at 37° C. for 18 hrs. [³H] thymidine (Perkin Elmer NET027A001MC) was then added to the cells at a final concentration of 3 uCi/mL and placed in an incubator at 37° C. for 24 hrs. The media was removed and the cells were washed with warmed PBS twice. 500 uL of warmed trypsin (Gibco cat# 25300-054) was added to each well and they were place in an incubator at 37° C. for 15 min. To precipitate the DNA, 500 uL of ice cold 20% TCA (MP Biomedicals cat# 152592) was added to each well. The resulting suspension was filtered using a vacuum manifold and glass fiber filters (Whatman cat# 1827-025). The fiber filters were then counted using a liquid scintillation counter (Wallac 1409). Results were normalized to the total signal of the challenge, graphed using Graphpad Prism (Ver. 5.00) and reported as % challenge stimulated proliferation.

### Results

The results are shown in FIG. 4. The results demonstrate that the tested Rho kinase inhibitors of Formula I or II compounds reduced the smooth muscle cell proliferation in vitro. The majority of the tested compounds decreased the proliferation to less than 50% of the normal rate at a concentration of 30 uM.

### Example 6

### Collagen-Induced Arthritis (CIA) in a Mouse Model

### Relevance

Collagen-induced arthritis (CIA) in the mouse has proven to be a useful model of RA because it exhibits clinical and histopathologic features similar to those of the human disease and demonstrates many of the cellular and humoral immunity characteristics found in human RA (Cuzzocrea et al. Arthritis & Rheumatism, 52:940-950, 2005 and Devesa et al. Arthritis & Rheumatism, 52:3230-3238, 2005). Additionally, the recruitment and activation of neutrophils, macrophages and lymphocytes into joint tissues and the formation of pannus are hallmarks of the pathogenesis of both CIA and human RA (Cuzzocrea et al. Arthritis & Rheumatism, 52:940-950, 2005).

### Protocol

DBA/1J mice (9-wk-old) are housed in a controlled environment and are provided with access to standard rodent laboratory food and water. On day 1, the animals are treated with type II collagen (CII), injected intradermally at the base of the tail as a 100 uL emulsion containing 100 ug of CII and Freund's complete adjuvant (CFA), and with a second injection of CII on day 21. The development of arthritis in mice is evaluated daily starting on day 20 after the first intradermal injection, using a macroscopic scoring system as follows: 0=no signs of arthritis; 1=swelling and/or redness of the paw or 1 digit; 2=2 joints involved; 3=more than 2 joints involved; 4=severe arthritis of the entire paw and digits. An arthritis index for each mouse is calculated by addition the scores from the 4 individual paws. Clinical severity is also determined by quantitating the change in paw volume by plethysmometry (Cuzzocrea et al. Arthritis & Rheumatism, 52:940-950, 2005). Compounds of this invention are dosed via i.p administration twice a day at the dose of 1 mg/kg to 100 mg/kg of body weight starting from days 22 to 29 and are sacrificed on day 30 after CIA induction. A control group of animals receives i.p saline.

### Histological Examination

On day 35, animals are anesthetized and killed, and paws and knees are removed and fixed in 10% formalin. The paws are then trimmed, placed in decalcifying solution for 24 hours, embedded in paraffin and sectioned at 5 um, stained with hematoxylin and eosin and studied using light microscopy. Arthritis damage (histologic damage score) is evaluated and scored by an investigator who is blinded with regard to the treatment regimen. Morphologic features are scored as: 0=no damage, 1=edema, 2=presence of inflammatory cells, 3=bone resorption,

### Measurement of Cytokines

Hind paws are amputated above the ankle and homogenized in 1 mL of 10 mM HEPES buffer, pH 7.4, containing 0.32M sucrose, 100 mM EDTA, 1 mM dithiothreitol, 2 mM phenylmethylsulfonyl fluoride, and 100 mM leupeptin. After centrifugation at 1,200 g for 15 minutes at 4C supernatants are removed and used for determination of cytokine levels, specifically TNF-α and IL-1β, by ELISA.

Quantification of Angiogenesis within the Joint

Endothelial cells are detected using a GSL-1 lectin immunohistochemical staining with the steptavidin-biotinperoxidase complex method. Knee joint slides are deparaffinized in xylene and dehydrated through serially diluted ethanol solutions down to distilled water. After blocking endogeneous peroxidase activity in blocking solutions for 1 hour, the slides are pretreated with blocking serum and then incubated with GSL-1 isolectin B4 for one hour at room temperature. Then, the slides are incubated with goat antibody to GSL-1 isolectin B4 for one hour, washed, and incubated with biotinylated rabbit antigoat immunoglobulins for 30 min in a moist chamber at room temperature. The samples are incubated with streptavidin-biotinperoxidase for 10 min using diaminobenzidine tertrahydrochloride as the chromogen. Between each step, the slides are washed three times for 5 min with TBS. They are then counterstained by incubation with hemalun for 40 sec and mounted with Glycergel. For each mouse, three non-serial sections from each knee are studied. For each section, five pictures are taken at low magnification, for example 400×. The area of the picture that is not in the synovium is subtracted from the total area. Any GSL-1 stained cell or group of cells with a lumen is considered as an individual vessel. Synovial vascular density is calculated as follows: each vessel in the synovium is counted and the number of vessels is divided by the synovium area. (Yin L et al. Mol Cancer Ther, 6: 1517-1525, 2007)

### Results

After day 30 after the first CII administration, the arthritis damage, edema, cellular influx, cytokine production, and degree of angiogenesis are measured and compared in the compound-treated mice vs. saline-treated mice. Improvements in at least one of the above-mentioned endpoints is observed.

### Example 7

### Mouse Model of TNBS-Induced Colitis as a Model of IBD

### Protocol

C57BL/10 male mice (6-8 weeks old) are used in studies of the acute form of TNBS-colitis. BALB/c female mice (8-10 weeks old) are used in studies of a chronic form of TNBS-colitis. TNBS (2,4,6-trinitrobenzene sulfonic acid) is a haptenation agent used to induce colitis.

Mice are lightly anesthetized with isoflurane and then administered a haptenating agent (either TNBS or oxazolone dissolved in ethanol) per rectum via a catheter equipped with a syringe; the catheter is then advanced into the rectum until the tip is 4 cm proximal to the anal verge at which time the haptenating agent is administered in a total volume of 150 μl. To ensure distribution of the haptenating agent within the entire colon and cecum, mice are held in a vertical position for 30 seconds after the intra-rectal injection. Control mice are administered an ethanol solution without haptenating agent using the same technique. 3 mg TNBS in 45% ethanol is used for studies of treatment of established acute TNBS-induced colitis and 1.5-2.5 mg TNBS (in increasing doses) in 45% ethanol is administered each week for studies of treatment of chronic TNBS-induced colitis.

### Inflammation

Rho Kinase inhibitor Compounds of Formula I or II are administered to examine prevention of nascent TNBS-Colitis. Colitis is induced in C57BL/10 mice, by intra-rectal instillation of TNBS in ethanol as described above and then, 4 hours later Rho Kinase inhibitor Compounds of Formula I or II are administered by instillation or intra-peritoneal injection at a dose of 1 mg/kg to 100 mg/kg by body weight after TNBS administration and again on day 1 and day 2 after TNBS administration.

### Fibrosis

Rho Kinase inhibitor Compounds of Formula I or II are administered to examine the prevention of development of colonic fibrosis in chronic TNBS-colitis. In this study, TNBS is administered by the intra-rectal route each week for 8 weeks to mice. On day 35 after initiation of TNBS administration, mice are assembled into weight-matched sub-groups for various types of treatment. Mice are treated with Rho Kinase inhibitor Compounds of Formula I or II either intra-rectally on days 37 and 44 or intra-peritoneally daily on days 37 to 39 and days 44 to 46 at a dose of 1 mg/kg to 100 mg/kg of body weight. A similar regimen is followed for mice treated with vehicle control.

### Histological Examination

Colons are fixed in 10% buffered formalin and embedded in paraffin. Paraffin-embedded colon sections are cut and then stained with H&E or by the Masson's trichrome method. For calculation of inflammation indices or for assessment of fibrosis in treated and control group of mice, the sections are read masked and evaluated according to a formerly published scoring system.

### Results

Following histological examination of the inflammation and fibrosis of the colon of control mice vs. mice treated with Rho Kinase inhibitor Compounds of Formula I or II, improvements in at least one of the above-mentioned endpoints is observed in the compound-treated mice.

### Example 8

### Murine Model of DSS-Induced Colitis

### Relevance

Dextran sulfate sodium (DSS)-induced colitis in mice shows reproducible morphological changes, which are very similar to those seen in patients with ulcerative colitis, or IBD (Hollenbach, E. et al FASEB J; 13:1550-2, 2004).

### Protocol

Female BALB/c mice (6-8 weeks old) are used in studies of colitis. Mice are weighed and placed into groups randomly. Histological scoring and clinical assessments of colitis are performed in a masked fashion. The mice are adapted for 3 days following arrival after which colitis is induced by addition of 3% DSS (dextran sodium sulfate; Sigma) to normal drinking water for one week. After week one, DSS addition to water is stopped. Treatment with 200 μl 0.9% NaCl or Rho Kinase inhibitor compound of Formula I or II at 1 mg/kg to 100 mg/kg body weight solution by intraperitoneal injection twice a day is administered beginning 60 hours after DSS treatment. Bowel tissue from untreated animals and animals treated with Rho Kinase inhibitor compound of Formula I or I are evaluated for the degree of edema, mucosal injury, and infiltration of inflammatory cells into the colonic bowel. (Hollenbach, E. et al. FASEB J; 13:1550-2, 2004.)

### Clinical and Histological Analysis

Colitis score is calculated by assigning scores based on parameters from the disease activity index (DAI). The range varies from 0 (healthy) to 4 (maximal activity of colitis). On days 3, 5, 7, 10, and 13, mice (n=7 in each group per day) are euthanized by CO₂ inhalation. Immediately following, the colon is quickly removed, opened longitudinally, and cleared of fecal matter. Colon samples are either fixed in 4% buffered formalin or embedded in paraffin and 4-μm-thick serial step sections are stained with hematoxylin-eosin (HE). HE stained colon samples are histologically scored on a scale of 0 to 4 for inflammation, extent, regeneration, and crypt damage.

### Histological Disease Score

Histological parameters of experimentally induced colitis and the effects of Rho Kinase inhibitor compound of Formula I or II are evaluated. Treatment of mice with DSS produces a mild colitis after three days with multiple erosive lesions and inflammatory cell infiltrations. The impairment of the glandular architecture and the infiltration of macrophages, lymphocytes, and occasional eosinophils and neutrophils between day 3 and 7 are evaluated.

### Differential White Blood Cell Count

Blood, around 0.4 mL, is drawn intracardially and mixed with 50 μl of 0.5 M EDTA. Blood samples are subjected to differential blood cell count analysis, including Monocytes and peripheral granulocytes, after induction of colitis starting at day 5 through day 13.

### Results

After discontinuation of DSS, the resolution of inflammation, mucosal injury, and the degree of edema in the colonic bowel are measured and compared in the saline-treated mice vs. the mice treated with a Rho Kinase inhibitor compound of Formula I or II. Improvements in at least one of the above-mentioned endpoints is observed.

### Example 9

### NIH/3T3 Cell Morphology Assay

### Relevance

The assay demonstrates that a compound's in vitro ROCK inhibition activity manifests itself in morphology changes, such as actin stress fiber disassembly and alteration in focal adhesions in intact cells leading to inhibition of acto-myosin driven cellular contraction. These morphology changes provide the basis for the beneficial pharmacological effects sought in the setting of the disease processes described in this application, specifically the disruption of the actin stress fibers and regulation of focal adhesions and its impact on cell mobility, remodeling and chemotaxis (Howard et al. The J. of Cell Biology 98:1265-1271, 1984); and vasopermeability, endothelial and epithelial permeability and associated edema (Stephens et al., Am. Rev. Respir. Dis. 137:4220-5, 1988 and Vandenbroucke et al., Ann. N. Acad. Sci. 1123: 134-145, 2008.)

### Protocol

NIH/3T3 cells were grown in DMEM-H containing glutamine and 10% Colorado Calf Serum. Cells were passaged regularly prior to reaching confluence. Eighteen to 24 hours prior to experimentation, the cells were plated onto Poly-L-Lysine-coated glass bottom 24-well plates. On the day of experimentation, the cell culture medium was removed and was replaced with the same medium containing from 10 nM to 25 μM of the test compound, and the cells were incubated for 60 minutes at 37° C. The culture medium was then removed and the cells were washed with warmed PBS and fixed for 10 minutes with warmed 4% paraformaldehyde. The cells were permeabilized with 0.5% Triton-X, stained with TRITC-conjugated phalloidin and imaged using a Nikon Eclipse E600 epifluorescent microscope to determine the degree of actin disruption. Results were expressed as a numerical score indicating the observed degree of disruption of the actin cytoskeleton at the test concentration, ranging from 0 (no effect) to 4 (complete disruption), and were the average of at least 2 determinations.

All compounds tested show measurable activity in the cell morphology assay, with most of the compounds providing substantial effects (score of ≧2 at 1 μM) on the actin cytoskeleton at the tested concentration (see Table 3).

TABLE 3
Cell Morphology Assay Data

| Compound | Cell score at 1 μM |
|---|---|
| 1.002 | 1.4 |
| 1.004 | 1.8 |
| 1.005 | 1.3 |
| 1.006 | 2 |
| 1.008 | 2 |
| 1.024 | 2.4 |
| 1.025 | 2 |
| 1.034 | 2 |
| 1.039 | 2 |
| 1.041 | 2.5 |
| 1.046 | 2.5 |
| 1.048 | 1.5 |
| 1.051 | 2.5 |
| 1.052 | 2.8 |
| 1.062 | 2.3 |
| 1.066 | 2 |
| 2.002 | 1.8 |
| 2.006 | 2.8 |
| 2.008 | 1 |
| 2.016 | 1.8 |
| 2.017 | 2 |
| 2.018 | 1.8 |
| 2.026 | 2 |

### Example 10

### In Vivo Anti-Inflammatory Activity

### Relevance

The mouse ovalbumin sensitization model has been developed by investigators to study malfunctioning of the immune system, cellular infiltration composed primarily of eosinophils and neutrophils, acute and chronic inflammation, fluid accumulation (edema), especially in asthma. Although this model is mostly utilized in the context of asthma, this model can be utilized to demonstrate the in vivo anti-inflammatory properties of Compounds of Formula I or II.

### Protocol

Male BALB/c mice were ordered from Charles River Laboratories (Raleigh, N.C.). The animals were approximately 19 to 21 grams at time of receipt. Upon arrival, the animals were randomized into groups of five males per cage and assigned to a dosing group. Animals were quarantined for 7 days under test conditions. They were observed daily for general health status and ability to adapt to the water bottles. Animals were sensitized on day 0 and 14 of study by an intraperitoneal injection with 20 μg of ovalbumin (ova) and 2.0 mg aluminum hydroxide (alum) which initiates the development of a specific T-helper (Th) cells type 2 resulting in asthmatic animals (denoted as Ova in the fugures). One group of animals received an injection of saline to use as control animals (denoted as normal in the figures). All animals were challenged with aerosolized 1% ova once daily for 25 minutes on days 28, 29, and 30 (Zosky, et al. Respiratory Research. 2004; 5:15). Aerosol challenge consists of using an Aerogen Aeroneb nebulizer and controller with a particle size of 4-6 μm mass median aerodynamic diameter (MMAD) with a distribution of 400 μl per minute. This aerosol challenge is necessary to target the Th2-driven allergic inflammation in the lower airways.

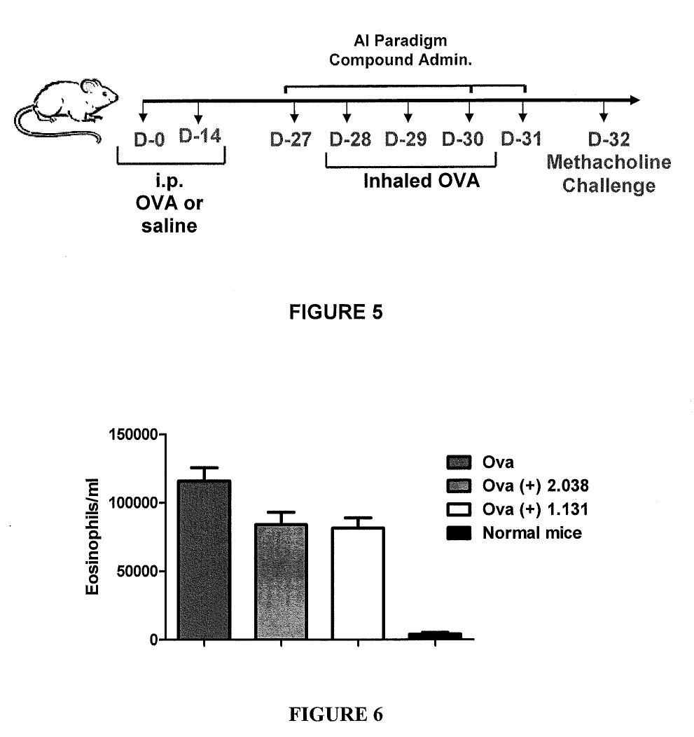

The anti-inflammatory dosing paradigm (FIG. 5) was utilized to evaluate the anti-inflammatory effects of experimental compounds. The anti-inflammatory dosing paradigm consists of dosing the animals once a day starting on day 27 and finishing on either day 30 or 31 (1 hr prior to the aerosolized ovalbumin challenges on days 28 to 30) but not on day 32 when hyperreactivity evaluation occurs (described in Example 11). On day 32 of the experiment, after measurement of airway hyperreactivity, BALF was collected and all animals were anesthetized, bled and euthanized.

Bronchoalveolar lavage fluid (BALF) was collected by infusing 3.0 ml of saline with 10% fetal calf serum into the lungs via the trachea and then withdrawing the fluid. The total amount of cells/ml of BALF fluid was determined via manual cell count on hemocytometer. The BALF was centrifuged, supernatant removed and analyzed for cytokine concentrations as described below, and cell pellet reconstituted in 500 μL of fluid. Cytospin slides were prepared from the cell pellet using 100 μL of fluid and spinning samples for 5 minutes at 5000 rpms in a cytospin centrifuge, Following Hema3 stain, relative percentages of individual leukocytes were determined on a 200 cell count for each sample. The final concentration of individual leukocyte cell types per ml of BALF was determined by multiplication of the relative percentage of individual leukocytes with the total amount of cells/ml of BALF fluid.

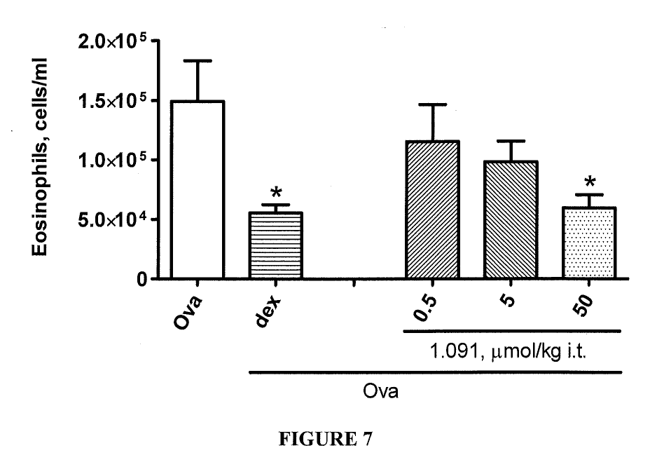

Evaluation of the differential counts performed on these samples showed an increased number of inflammatory cells in the ova-sensitized, ova-challenged animals. FIG. 6 shows the eosinophils per ml of BALF in ova-sensitized, ova-challenged mice, mice treated with Compound 2.038, mice treated with Compound 1.131 and normal mice. Compounds were dosed orally to day 31 according to the anti-inflammatory dosing paradigm shown in FIG. 5. Airway eosinophil infiltration was reduced in animals treated with the two tested compounds (FIG. 6). As shown in FIG. 7, Compound 1.091 generates a reduction of eosinophils when dosed i.t. to day 30 according to the anti-inflammatory dosing paradigm shown in FIG. 5.

The concentrations of cytokines in the BALF samples were determined using commercially available Bio-plex kits (Bio-Rad) for the detection of mouse IL-5, IL-13, and Eotaxin. The analysis of cytokine levels was measured using the Bio-Plex 200 (Bio-Rad) system according to the manufacturer's instructions. Substantial evidence suggests that cytokines play an important role in orchestrating and regulating inflammatory processes through the involvement of T-helper type 2 lymphocytes.

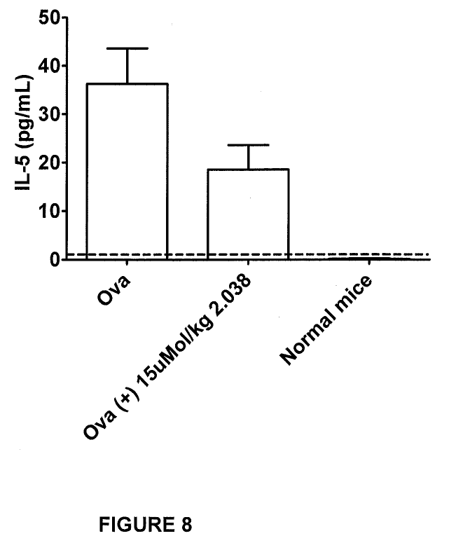

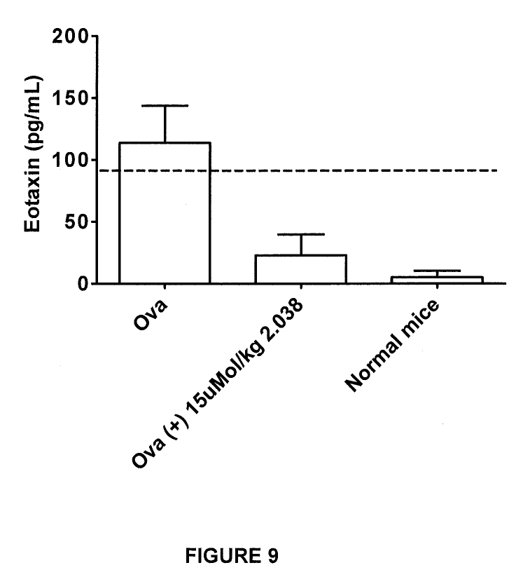

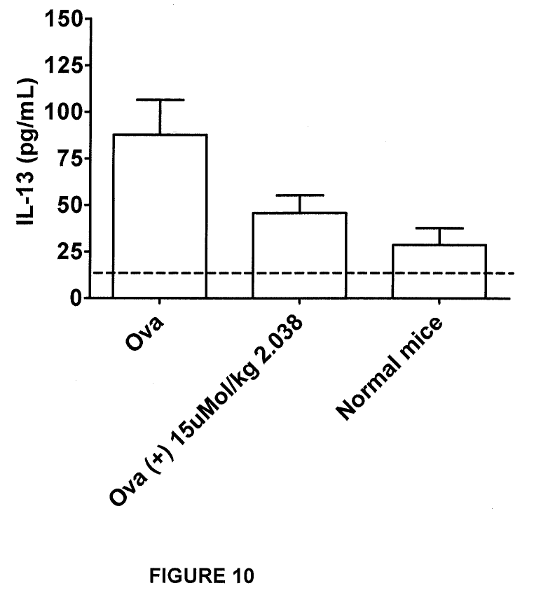

FIGS. 8-10 show the concentration of IL-5, Eotaxin, and IL-13 in (1) ova-sensitized, ova-challenged mice, (2) ova-sensitized, ova-challenged mice treated with Compound 2.038 (15 μmol/kg/oral on days 27 to 31), and (3) normal, saline-sensitized mice. The results showed that ova-sensitized, ova-challenged mice treated with Compound 2.038 had reduced levels of IL-5, Eotaxin, and IL-13.

### Example 11

### Prevention of Airway Hyperreactivity Development Via Decrease in Inflammation

### Relevance

Airway hyperreactivity is a downstream physiologic effect of inflammation in the mouse ovalbumin sensitization model. The objective of the experiment was to answer whether the decrease in inflammation due to ROCK inhibitor anti-inflammatory dosing results in the prevention of downstream physiological consequences as measured by Penh. Although this concept is demonstrated in a model of airway hyperreactivity due to pulmonary inflammation, these data support the general use of these compounds as anti-inflammatory agents to prevent the downstream physiological consequences of inflammation in an in vivo model.

### Protocol

Mouse model of ovalbumin sensitization was created as described in Example 10, The anti-inflammatory dosing paradigm (FIG. 5) was utilized to evaluate the prevention of airway hyperreactivity due to the anti-inflammatory effects of experimental compounds. The anti-inflammatory dosing paradigm consists of dosing the animals once a day starting on day 27 and finishing on either day 30 or 31 (1 hr prior to the aerosolized ovalbumin challenges on days 28 to 30) but not on day 32 when hyperreactivity evaluation occurs. On day 32 of the experiment, airway hyperreactivity was evaluated by placing conscious, unrestrained animals in a whole body plethysmometer (Buxco Wilmington, N.C.) and exposing them to escalating doses of nebulized methacholine, a known bronchial constrictor which acts through the muscarinic receptors of the lungs, (doses: 0.325-50 mg/ml). Exposure to the methacholine doses consisted of a 3 minute period during which a nebulizer was aerosolizing the methacholine and an additional 3 minute period following the cessation of nebulization. Over this 6 minute period, the plethysmometer monitors and generates numerical values for all parameters of the breath pattern. Enhanced pause (Penh), a unitless index of airway hyperreactivity, is derived from the expiratory side of the respiratory waveform measured via the plethysmograph and is used as an indirect measure of airway resistance and hyperreactivity. Penh is an indicator of changes in resistance within the airways and has been shown to be a valid marker for airway responsiveness to allergen challenge (Hamelmann, et al. Am J Respir Crit Care Med. 1997; 156:768-775). Following the methacholine dose response, BALF was collected and all animals were anesthetized, bled and euthanized.

### Statistical Methods

Within each experiment, a mouse was given a single compound and exposed to increasing doses of methacholine [0 (baseline), 0.375, 0.75, 1.5, 3, 6, 12, 25, 50 mg/ml]. The Penh value at each of the dose levels of methacholine represents the 6-minute average response. Change from baseline (CFB) in Penh was calculated at each methacholine dose and the area under the curve (AUC) for these CFB values was calculated using the trapezoidal rule. This same approach was applied for each mouse across multiple experiments.

For statistical analyses, a linear mixed-effects model where the response was the log 10 transformed value of AUC described above was used. Data from equal experimental conditions across experiments performed on different days were pooled for statistical analysis and data reporting. The various compounds were compared adjusting for the log 10-transformed baseline value of Penh and the chamber (1 of 10) of the plethysmometer each mouse was contained in during an experiment. A random intercept for each experiment was assumed to account for possible similarities of the results obtained from a given experiment (i.e., as a “blocking effect”). Pairwise comparisons of the compounds were performed using approximate t-tests to test the null hypothesis of no compound difference of the least-squares means of log 10(AUC). p values of less than 0.05 were considered statistically significant Computations were performed using PROC MIXED (SAS Version 9.1).

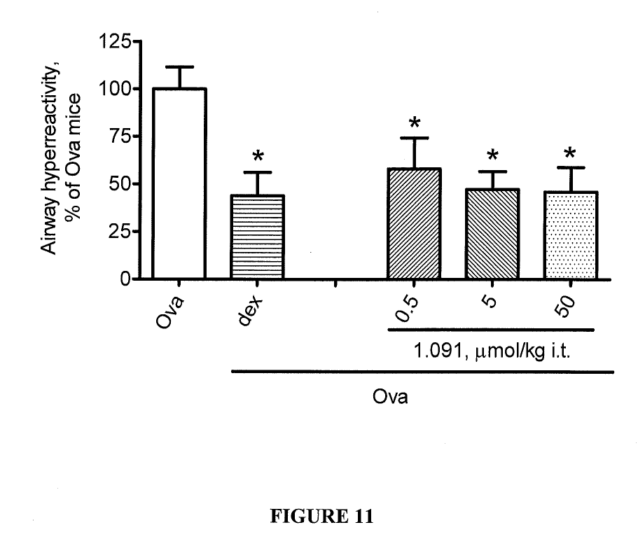

For Table 4, Penh values are reported as log 10 transformed AUC values. For FIG. 11, linear AUC values from compound treated mice were reported as a percent of linear AUC values from vehicle-treated ovalbumin-sensitized/ovalbumin-challenged (asthmatic) mice.

The oral administration of 15 μMol/kg of Compound 1.131 or 2.038 once a day during days 27 to 31 resulted in prevention of airway hyperreactivity to metacholine dosed on Day 32 (Table 4). As shown in FIG. 11 and Table 4, intratracheal administration of Compound 1.091 once a day during days 27 to 30 (FIG. 11) or Compounds 1.161, 2.066 or 2.059 once a day during days 27 to 31 (Table 4) according to the anti-inflammatory dosing paradigm shown in FIG. 5 resulted in prevention of airway hyperreactivity. Compound 1.091, 1.161, 2.066 or 2.059 had similar efficacy to dexamethasone, a corticosteroid anti-inflammatory control. These data support the use of these compounds to prevent the downstream physiologic consequences of inflammation.

TABLE 4
Anti-inflammatory dosing: Statistical Analysis of the AUC for
Average Penh Values Determined During Experiment
Normalized to Baseline for Each Animal

| | | Number | | | |
|---|---|---|---|---|---|
| | Dosing | of | | | |
| | concentration/ | animals | log10A | Student | |
| | route of | per | UC | Standard | t-test |
| | administration | group | (Penh) | Error | p-value |
| asthmatic | Vehicle/oral | 70 | 2.3354 | 0.04751 | |
| 1.131 | 15 μmol/kg/ | 10 | 2.0674 | 0.1061 | 0.0133 |
| | oral | | | | |
| 2.038 | 15 μmol/kg/ | 20 | 1.8981 | 0.07966 | <0.0001 |
| | oral | | | | |
| 1.161 | 0.5 μmol/kg/ | 10 | 2.0405 | 0.1083 | 0.0077 |
| | intratracheal | | | | |
| 2.066 | 0.5 μmol/kg/ | 10 | 2.0248 | 0.1091 | 0.0055 |
| | intratracheal | | | | |
| 2.059 | 0.5 μmol/kg/ | 10 | 1.9979 | 0.1084 | 0.0024 |
| | intratracheal | | | | |
| Y-27632 | 30 μmol/kg/ | 10 | 1.9942 | 0.1062 | 0.0017 |
| | oral | | | | |
| Dexamethasone | 1 mg/kg/oral | 30 | 2.0216 | 0.06546 | <0.0001 |
| non-asthmatic | Vehicle/oral | 20 | 1.7810 | 0.07973 | <0.0001 |

Compounds were administered on days 27 to 31 according to the anti-inflammatory dosing paradigm. The t-test was conducted for the comparison of compound-treated to vehicle-treated “asthmatic groups” based on the vehicle which was run in every study.

### Example 12

### Human Monocyte Cytokine Secretion Assay

### Relevance:

This assay demonstrates a compound's ability to inhibit the secretion of multiple pro-inflammatory cytokines from human monocytes. Reduction in the levels of pro-inflammatory cytokines is associated with improvement in disorders with an inflammatory component.

### Protocol

Peripheral blood from healthy human volunteers was collected and the monocytes isolated via Ficoll-paque density centrifugation. Monocytes were purified via an Easy Sep© Monocyte Enrichment Kit (Product number 19059) according to the manufacturer's instructions. The purified monocytes were then plated in 96-well plates at a density of 300,000 cells/mL in RPMI 1640+10% heat inactivated FBS media. The cells were allowed to pre-incubate with test compound at the indicated concentration for 30 minutes (37° C., 5% CO₂, humidified air); after which the supernatant was removed and media containing compound and 1 ng/mL LPS was added. Cells were allowed to incubate with compounds and LPS for 4 hours at 37° C. after which the supernatant was removed and stored at −80° C. Cytokine concentrations in the supernatant were determined using commercially available Bio-Rad Bio-Plex™ kits according the manufacturer's instructions.

### Results:

Compounds of Formulae I and II inhibit the release of multiple cytokines from human monocytes when incubated at 10 μM concentration in vitro, as shown in Table 5. Shown further in Table 6, potency determinations on compounds 2.059 and 2.066, both potent inhibitors of ROCK1 and ROCK2 and both of the chemical class in which R₂ is R₂-2, dose-dependently reduced the secretion of IL-1β, TNF-α and IL-9 from LPS-stimulated human monocytes, with potencies ranging from approximately 170 nM to 1 μM.

TABLE 5
Percent inhibition values for inhibition of cytokine secretion
at 10 μM of test compound

| Compound | IL-1β % | IL-6 % | TNF-α % |
|---|---|---|---|
| 1.072 | 98.2 | 96.1 | 83.8 |
| 1.074 | 43.9 | 96.0 | 87.7 |
| 1.075 | 49.7 | 73.9 | 51.6 |
| 1.076 | 51.0 | 81.2 | 78.9 |
| 1.077 | 30.3 | 43.3 | 52.3 |
| 1.078 | 60.4 | 111.0 | 88.1 |
| 1.079 | 59.3 | 31.1 | 56.5 |
| 1.091 | 165.5 | 108.2 | 104.6 |
| 1.093 | 109.0 | 49.7 | 76.1 |
| 1.106 | 121.5 | 95.0 | 80.6 |
| 1.107 | 111.3 | 122.1 | 83.1 |
| 1.108 | 131.3 | 89.8 | 116.7 |
| 1.109 | 190.5 | 312.9 | 118.3 |
| 1.110 | 133.6 | 111.7 | 118.6 |
| 1.123 | 82.6 | 64.7 | 62.7 |
| 1.124 | 99.5 | 101.4 | 61.5 |
| 1.127 | 198.0 | 67.3 | 97.3 |
| 1.131 | 48.3 | 68.6 | 85.2 |
| 1.132 | 58.6 | 72.5 | 80.3 |
| 1.133 | 54.5 | 70.7 | 66.2 |
| 1.134 | 43.2 | 74.6 | 69.1 |
| 1.135 | 57.0 | 123.2 | 108.0 |
| 1.136 | 66.3 | 95.0 | 71.5 |
| 1.137 | 40.3 | 46.2 | 58.0 |
| 1.138 | 257.4 | 76.6 | 130.9 |
| 1.141 | 50.4 | 71.7 | 75.7 |
| 1.142 | 82.8 | 40.7 | 68.6 |
| 1.143 | 76.8 | 130.5 | 66.4 |
| 1.145 | 129.2 | 95.1 | 88.9 |
| 1.146 | 85.2 | 128.0 | 97.7 |
| 1.148 | 63.9 | 78.6 | 56.1 |
| 1.149 | 69.8 | 121.5 | 119.9 |
| 1.150 | 78.2 | 89.2 | 94.4 |
| 1.151 | 84.5 | 114.1 | 88.9 |
| 1.152 | 74.7 | 94.7 | 120.1 |
| 1.153 | 64.1 | 106.2 | 74.3 |
| 1.154 | 52.3 | 104.4 | 86.4 |
| 1.155 | 76.7 | 121.8 | 79.7 |
| 1.156 | 60.7 | 92.5 | 70.5 |
| 1.157 | 121.4 | 92.6 | 65.1 |
| 1.158 | 80.8 | 133.1 | 86.6 |
| 1.159 | 97.1 | 84.8 | 76.1 |
| 1.161 | 87.7 | 86.3 | 153.5 |
| 1.162 | 95.5 | 99.8 | 158.7 |
| 1.163 | 166.7 | 140.9 | 91.6 |
| 1.164 | 80.1 | 109.5 | 89.0 |
| 1.165 | 129.9 | 114.3 | 103.5 |
| 1.166 | 107.0 | 87.2 | 82.2 |
| 1.170 | 80.6 | 72.7 | 67.8 |
| 1.171 | 78.9 | 91.8 | 72.2 |
| 1.173 | 86.1 | 79.5 | 80.1 |
| 1.175 | 29.3 | 38.2 | 47.4 |
| 1.176 | 95.2 | 112.4 | 72.4 |
| 1.183 | 68.7 | 123.3 | 76.5 |
| 1.185 | 39.8 | 63.0 | 66.6 |
| 1.186 | 64.1 | 105.3 | 68.2 |
| 1.195 | 115.4 | 94.4 | 67.7 |
| 1.197 | 179.1 | 128.8 | 83.3 |
| 1.200 | 0.0 | 0.0 | 0.2 |
| 1.206 | 88.7 | 164.0 | 97.3 |
| 1.208 | 62.0 | 109.0 | 92.0 |
| 1.212 | 116.3 | 111.0 | 108.1 |
| 1.213 | 111.1 | 81.7 | 77.4 |
| 1.215 | 136.7 | 63.2 | 60.4 |
| 1.217 | 118.6 | 73.8 | 71.3 |
| 1.219 | 138.9 | 127.7 | 82.1 |
| 1.223 | 117.0 | 88.5 | 60.7 |
| 1.226 | 99.3 | 52.2 | 66.6 |
| 1.227 | 69.4 | 66.7 | 79.3 |
| 1.229 | 44.9 | 63.2 | 50.7 |
| 1.233 | 78.5 | 78.9 | 79.0 |
| 1.236 | 75.2 | 93.0 | 98.0 |
| 1.237 | 97.1 | 100.9 | 70.6 |
| 1.238 | 101.1 | 62.9 | 73.2 |
| 1.239 | 39.4 | 84.7 | 58.5 |
| 1.246 | 103.0 | 108.3 | 79.0 |
| 1.249 | 133.8 | 56.2 | 60.0 |
| 1.252 | 139.2 | 68.3 | 101.6 |
| 1.253 | 160.6 | 228.6 | 126.8 |
| 1.258 | 104.1 | 83.5 | 94.0 |
| 1.262 | 145.7 | 156.6 | 135.3 |
| 2.026 | 166.0 | 180.7 | 109.1 |
| 2.031 | 49.0 | 89.3 | 66.4 |
| 2.038 | 90.8 | 79.7 | 70.2 |
| 2.039 | 49.8 | 70.3 | 47.8 |
| 2.054 | 24.0 | 56.8 | 37.9 |
| 2.058 | 1.2 | 1.3 | 10.6 |
| 2.059 | 0.3 | 0.0 | 6.9 |
| 2.060 | 5.9 | 19.6 | 33.0 |
| 2.064 | 14.3 | 45.7 | 66.2 |
| 2.066 | 0.0 | 0.0 | 25.2 |

TABLE 6
IC₅₀ values for inhibition of cytokine secretion

| | IL-1β (nM) | TNF-α (nM) | IL-9 (nM) |
|---|---|---|---|
| Compound 2.059 | 169.4 ± 13.0 | 207.1 ± 17.0 | 268.6 ± 28.1 |
| Compound 2.066 | 346.2 ± 182.3 | 610.6 ± 154.1 | 934.9 ± 407.5 |

### Example 13

### LPS-Induced Neutrophilia and Cytokine Production Assay

### Relevance

Marked neutrophilia can occur upon tissue inflammation. The LPS-induced neutrophilia model is often used to determine the potential efficacy of therapeutic approaches to limit inflammatory responses. This assay is an in vivo assay of neutrophil accumulation and cytokine production that can be used to evaluate the activity of Rho Kinase inhibitor compounds of Formula I or II as anti-inflammatory agents in a whole animal model. Neutrophil accumulation and cytokine production is indicative of the inflammatory response and the activity of compounds to decrease neutrophil accumulation and cytokine production in this assay supports the use of these compounds to treat disorders with an inflammatory component, such as RA and IBD.

### Protocol

Male BALB/c mice, approximately 19 to 21 grams, were ordered from Charles River Laboratories (Raleigh, N.C.). All animals were challenged with aerosolized LPS (10 μg/ml) for 25 minutes on study day 0. LPS aerosol was generated using an Aerogen Aeroneb nebulizer and controller providing a flow of 400 μl/min and a particle size of 2-4 μm MMAD. Rolipram was administered i.p at 20 mg/kg. Compound 1.091 or Compound 2.059 was administered intratracheally (i.t.) at 0.5-50 μmol/kg body weight one hour prior to LPS challenge. Four hours following LPS challenge, BALF was collected using a total of 3 ml of 0.9% sodium chloride containing 10% fetal calf serum. Total cell counts were determined using the Coulter Counter. For differential evaluations, BALF was centrifuged and cytospin slides prepared and stained with Hema3 stain. Manual leukocyte counts were then completed on 200 cells. The final concentration of individual leukocyte cell types per ml of BALF was determined by multiplication of the relative percentage of individual leukocytes with the total amount of cells/ml of BALF fluid. The concentration of IL-1β in the BALF samples was determined using commercially available Bio-plex kits (Bio-Rad). The analysis of cytokine levels was measured using the Bio-Plex 200 (Bio-Rad) system according to the manufacturer's instructions.

### Results

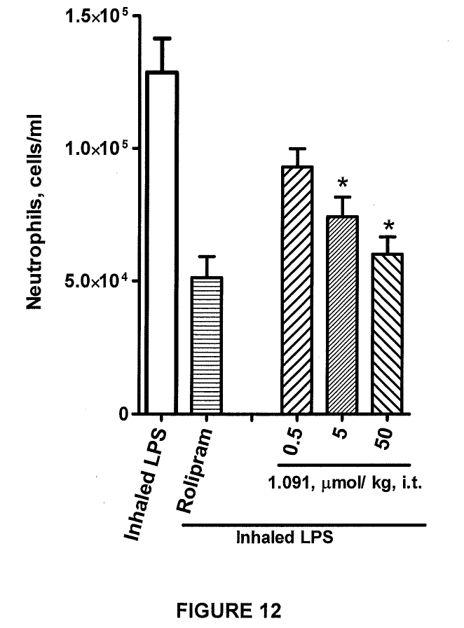

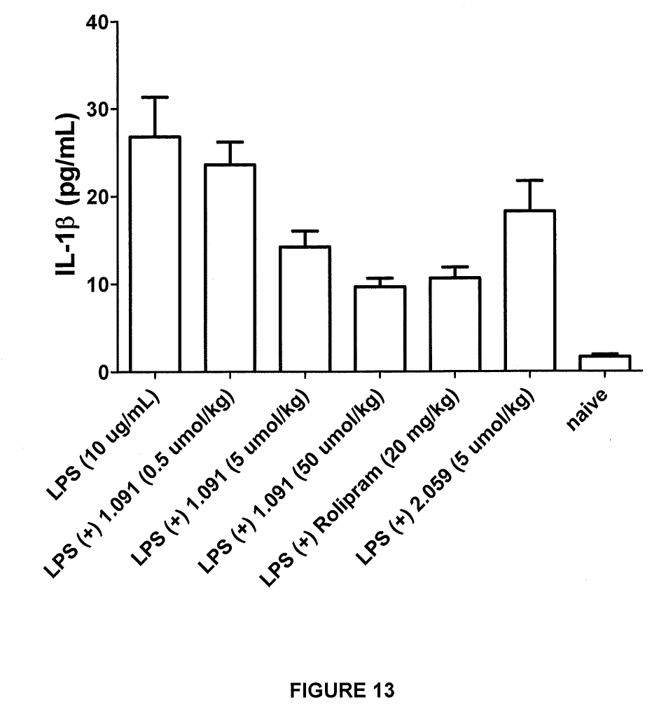

FIG. 12 shows a significant reduction in pulmonary neutrophilia influx after intratracheal dosing of Compound 1.091. The efficacy of Compound 1.091 when dosed intratracheally is similar to the efficacy of the control compound rolipram dosed i.p. FIG. 13 shows the reduction in IL-1β after intratracheal administration of Compound 1.091 or Compound 2.059. These data demonstrate the efficacy of Rho kinase inhibitors of Formula I or II to inhibit inflammation in vivo.

### Example 14

### PDGF-Stimulated Smooth Muscle Cell Proliferation Assay

### Relevance:

This assay demonstrates a compound's ability to inhibit cellular proliferation induced by platelet derived growth factor (PDGF). Activity of compounds in the assay demonstrates the anti-proliferative properties of these compounds and supports the use of these compounds in the treatment of disorders associated with a proliferative component.

### Protocol

Effects on cell proliferation were measured using a bromodeoxyuridine (BrdU) incorporation assay. A-10 rat thoracic aorta cells (ATCC #CRL 1476) were plated at 1000 cells per well in 96-well plates in Dulbecco's Modified Eagles Medium-High Glucose (Gibco cat. # 11995-065) containing 10% Fetal Bovine Serum (Sigma EC# 232-690-6) and allowed to grow for 24 hrs in an incubator at 37° C. Growth media was then removed and the cells were washed with warmed PBS (Gibco cat# 14190-144). Serum free media containing 0.1% BSA was added to the cells. 24 hours later the media was removed and replaced with warmed serum free media. Cells were treated with either 1 μM or 10 μM of test compound and incubated for 60 min at 37° C. prior to the addition of 10 ng/mL PDGF (BD Biosciences cat. # 354051) and placed in an incubator at 37° C. for 18 hrs with both compound and stimulant present. Proliferation was then monitored using the BrdU Cell Proiferation Assay, HTS (Calbiochem cat. # HTS01). BrdU was allowed to incorporate into cells for 24 hours prior to the addition of fixative/denaturing solution and the fluorometric detection of incorporated BrdU using a BrdU antibody as per manufacturer's directions. Data are reported as a percent of the PDGF-stimulated BrdU incorporation.

### Results:

As shown in Table 7, compounds of Formulae I and II reduced PDGF-stimulated proliferation of A10 cells with efficacy ranging from 10-80% inhibition when dosed in vitro at 1 μM.

TABLE 7
Reduction of PDGF-stimulated proliferation of A-10 cells as a
percent of the total challenge-stimulated proliferation.

| | Percent of | Percent of | Percent of | Percent of |
|---|---|---|---|---|
| | PDGF | PDGF | PDGF | PDGF |
| | Induced | Induced | Induced | Induced |
| | Proliferation | Proliferation | Proliferation | Proliferation |
| | at 10 μM | at 10 μM | at 1 μM | at 1 μM |
| Compound | Avg | SEM | Avg | SEM |
| 1.074 | 46.9 | 3.5 | 79.9 | 9.7 |
| 1.076 | 53.7 | 4.1 | 84.0 | 8.5 |
| 1.091 | 69.3 | 5.5 | 85.7 | 5.3 |
| 1.108 | 43.7 | 1.6 | 83.1 | 6.7 |
| 1.124 | 61.6 | 2.6 | 68.5 | 3.1 |
| 1.131 | 36.6 | 2.4 | 61.7 | 4.8 |
| 1.132 | 30.3 | 1.3 | 48.9 | 3.4 |
| 1.135 | 35.0 | 3.9 | 52.6 | 4.9 |
| 1.136 | 39.8 | 2.6 | 71.4 | 1.3 |
| 1.138 | 27.0 | 1.7 | 46.3 | 1.5 |
| 1.148 | 63.5 | 3.0 | 56.9 | 2.7 |
| 1.151 | 63.8 | 4.1 | 51.0 | 2.1 |
| 1.161 | 33.4 | 0.9 | 50.0 | 3.7 |
| 1.162 | 42.5 | 1.6 | 55.6 | 2.3 |
| 1.165 | 57.9 | 1.2 | 74.8 | 6.1 |
| 1.167 | 52.7 | 4.6 | 78.8 | 4.5 |
| 1.173 | 35.8 | 2.8 | 55.4 | 4.2 |
| 1.175 | 49.0 | 2.5 | 58.2 | 2.3 |
| 1.180 | 64.8 | 5.0 | 92.4 | 7.9 |
| 1.197 | 48.9 | 2.8 | 52.5 | 1.5 |
| 1.204 | 42.8 | 5.3 | 79.3 | 3.0 |
| 1.206 | 51.1 | 2.1 | 77.5 | 5.8 |
| 1.213 | 52.3 | 3.6 | 70.1 | 2.3 |
| 1.215 | 54.0 | 5.3 | 70.8 | 4.0 |
| 1.237 | 51.4 | 4.8 | 63.5 | 5.2 |
| 1.238 | 48.6 | 3.2 | 40.7 | 1.9 |
| 1.239 | 37.8 | 1.6 | 41.7 | 2.7 |
| 1.253 | 47.9 | 2.0 | 44.8 | 3.1 |
| 1.258 | 43.4 | 4.7 | 50.5 | 3.3 |
| 2.009 | 56.5 | 3.9 | 128.9 | 13.4 |
| 2.022 | 39.4 | 1.1 | 89.7 | 4.5 |
| 2.025 | 68.0 | 4.1 | 69.8 | 4.6 |
| 2.026 | 52.0 | 2.5 | 74.5 | 6.5 |
| 2.027 | 64.4 | 5.8 | 79.4 | 5.6 |
| 2.031 | 52.6 | 2.8 | 90.3 | 9.9 |
| 2.038 | 62.7 | 3.5 | 58.6 | 1.2 |
| 2.041 | 61.5 | 3.1 | 81.8 | 4.8 |
| 2.046 | 32.1 | 1.4 | 57.4 | 1.2 |
| 2.047 | 53.8 | 3.2 | 65.3 | 3.0 |
| 2.054 | 84.6 | 6.4 | 68.2 | 4.0 |
| 2.059 | 25.5 | 1.1 | 75.0 | 5.7 |
| 2.064 | 56.2 | 3.9 | 53.1 | 1.9 |
| 2.066 | 19.8 | 0.7 | 20.0 | 0.7 |

### Example 15

### Akt3 and p70S6K Inhibition Assay

### Relevance:

This assay demonstrates a compound's ability to inhibit the kinases Akt3 and p70S6K in vitro. Both kinases are known to play a role in proliferation pathways.

### Protocol

Inhibition of Akt3 and p70S6K activity was determined using the IMAP™ FP Progressive Binding Kit (Molecular Devices product number R8127). Akt3 human enzyme (Upstate Chemicon #14-502), or p70S6K human enzyme (Upstate Chemicon #14-486), and Flourescein tagged substrate peptide (Molecular Devices product number R7110) or (Molecular Devices product number R7184), for Akt3 and p70S6K respectively, was pre-incubated with test compound for 5 minutes in buffer containing 10 mM Tris-HCL pH 7.2, 10 mM MgCl₂, 1 mM DTT and 0.1% BSA. Following the pre-incubation, 30 μM ATP was added to initiate the reaction. After 60 minutes at RT, Molecular Devices IMAP™ binding solution was added to bind phosphorylated substrate. After 30 minutes of incubation in the presence of the IMAP™ beads the fluorescence polarization was read and the ratio was reported as mP. IC₅₀ results were calculated using the Prism software from Graphpad. The Kᵢ values were determined according to the following formula: Kᵢ=IC₅₀/(1+([ATP Challenge]/EC₅₀ ATP)).

### Results:

As shown in Table 8, many compounds of Formulae I and II show sub-micromolar inhibitory potencies against both Akt3 and p70S6K.

TABLE 8
Akt3 and p70S6K potency data

| | | Akt3 Ki, | p70S6K Ki, | p70S6K Ki, |
|---|---|---|---|---|
| | Akt3 Ki, Avg, | StdDev, | Avg, | StdDev, |
| Compound | nM | nM | nM | nM |
| 1.072 | 4752.1 | 617.1 | 1130.3 | 263.7 |
| 1.074 | 437.4 | 13.2 | 548.3 | 170.9 |
| 1.075 | 5321.5 | 61.8 | 974.6 | 166.8 |
| 1.076 | 240.9 | 6.2 | 414.3 | 162.7 |
| 1.077 | 5253.2 | 1422.9 | 715.5 | 291.5 |
| 1.078 | 3267.4 | 150.9 | 1678.1 | 640.4 |
| 1.079 | 7191.7 | 445.6 | 3012.8 | 963.8 |
| 1.091 | 5388.5 | 171.6 | 1420.4 | 78.5 |
| 1.093 | 1824.9 | 27.9 | 2025.6 | 356.8 |
| 1.106 | 3914.9 | 257.1 | 1329.1 | 268.0 |
| 1.107 | 16304.0 | 1575.9 | 3356.5 | 701.7 |
| 1.108 | 205.0 | 2.2 | 510.6 | 106.0 |
| 1.109 | 5190.9 | 318.3 | 2495.5 | 314.8 |
| 1.110 | 462.6 | 2.3 | 1298.2 | 175.9 |
| 1.123 | 2406.9 | 287.1 | 2810.7 | 597.6 |
| 1.124 | 7868.0 | 909.4 | 3325.3 | 542.0 |
| 1.127 | 975.4 | 126.4 | 2065.5 | 54.3 |
| 1.131 | 282.6 | 2.0 | 502.8 | 112.4 |
| 1.132 | 81.8 | 8.2 | 514.6 | 111.1 |
| 1.133 | 148.3 | 3.7 | 531.8 | 45.6 |
| 1.134 | 150.7 | 22.1 | 519.7 | 81.1 |
| 1.135 | 444.2 | 32.9 | 588.6 | 142.4 |
| 1.136 | 289.7 | 12.5 | 1236.7 | 413.1 |
| 1.137 | 197.9 | 10.3 | 353.6 | 132.2 |
| 1.138 | 91.3 | 48.3 | 443.5 | 36.3 |
| 1.141 | 1263.0 | 133.1 | 387.5 | 5.8 |
| 1.142 | 8268.5 | 702.6 | 2524.8 | 882.2 |
| 1.143 | 706.5 | 130.5 | 538.2 | 173.7 |
| 1.145 | 1190.5 | 63.5 | 2296.4 | 602.2 |
| 1.146 | 204.9 | 24.7 | 741.5 | 272.3 |
| 1.148 | 1131.4 | 161.7 | 435.5 | 138.0 |
| 1.149 | 7395.9 | 410.0 | 1888.4 | 661.8 |
| 1.150 | 3183.1 | 98.7 | 1273.8 | 106.7 |
| 1.151 | 708.9 | 112.8 | 530.7 | 69.6 |
| 1.152 | 1976.2 | 155.8 | 523.5 | 295.5 |
| 1.153 | 9950.2 | 2150.4 | 2376.1 | 553.3 |
| 1.154 | 4947.5 | 541.2 | 1130.1 | 355.3 |
| 1.155 | 5680.5 | 644.8 | 1751.6 | 502.8 |
| 1.156 | 8772.6 | 427.6 | 3244.6 | 675.0 |
| 1.157 | 29192.3 | 10235.1 | 8693.4 | 2357.4 |
| 1.158 | 5905.2 | 343.4 | 1971.7 | 454.0 |
| 1.159 | 1232.9 | 459.5 | 2061.8 | 271.7 |
| 1.161 | 63.5 | 3.6 | 129.4 | 73.5 |
| 1.162 | 92.0 | 0.9 | 387.4 | 217.4 |
| 1.163 | 4423.8 | 182.3 | 1875.2 | 496.6 |
| 1.164 | 4306.8 | 26.6 | 1957.4 | 729.2 |
| 1.165 | 4140.0 | 293.7 | 1627.1 | 584.4 |
| 1.166 | 18132.9 | 4816.3 | 5163.5 | 1419.0 |
| 1.167 | 8247.3 | 802.7 | 1071.0 | 516.6 |
| 1.170 | 7814.3 | 82.1 | 2046.3 | 580.9 |
| 1.171 | 9326.9 | 448.0 | 3419.0 | 841.6 |
| 1.173 | 157.0 | 0.5 | 339.7 | 204.4 |
| 1.175 | 2820.2 | 294.6 | 853.0 | 92.0 |
| 1.176 | 20941.5 | 4664.9 | 8755.7 | 3209.3 |
| 1.178 | 711.4 | 5.8 | 1116.2 | 637.4 |
| 1.180 | 12022.9 | 416.9 | 1029.2 | 139.1 |
| 1.183 | 9007.8 | 1662.8 | 2477.1 | 1431.3 |
| 1.185 | 4216.6 | 403.6 | 1152.2 | 761.8 |
| 1.186 | 10237.7 | 1867.1 | 1612.5 | 982.8 |
| 1.195 | 21975.8 | 379.4 | 2731.0 | 1192.9 |
| 1.197 | 64051.2 | 47694.4 | 8688.8 | 366.2 |
| 1.200 | 10608.5 | 131.2 | 3903.1 | 3979.1 |
| 1.204 | 1908.2 | 34.3 | 926.8 | 122.9 |
| 1.206 | 529.1 | 22.0 | 314.4 | 209.6 |
| 1.208 | 345.7 | 19.4 | 720.6 | 705.8 |
| 1.212 | 390.2 | 3.8 | 894.0 | 580.3 |
| 1.213 | 3207.8 | 140.6 | 2097.2 | 112.7 |
| 1.215 | 14753.0 | 1613.1 | 1285.8 | 108.5 |
| 1.217 | 10301.1 | 93.6 | 3501.9 | 3691.2 |
| 1.219 | 38297.7 | 11679.7 | 4969.9 | 1893.5 |
| 1.223 | 11139.0 | 1467.2 | 3101.9 | 1629.9 |
| 1.226 | 531.0 | 1.1 | 1348.5 | 1389.6 |
| 1.227 | 3476.0 | 196.6 | 1580.9 | 623.5 |
| 1.229 | 24557.8 | 17008.1 | 3128.5 | 322.4 |
| 1.233 | 2628.6 | 182.4 | 2004.9 | 815.1 |
| 1.236 | 3716.5 | 474.9 | 2755.4 | 2914.8 |
| 1.237 | 7910.2 | 217.5 | 9873.2 | 7272.6 |
| 1.238 | 4171.1 | 173.1 | 2609.6 | 1573.2 |
| 1.239 | 17657.7 | 4393.7 | 10026.9 | 8534.5 |
| 1.246 | 1096.1 | 9.5 | 1879.2 | 1883.4 |
| 1.249 | 1599.7 | 63.8 | 937.5 | 226.8 |
| 1.252 | 205.0 | 11.9 | 170.7 | 84.1 |
| 1.253 | 2597.1 | 29.9 | 2515.0 | 1464.8 |
| 1.258 | 315.2 | 94.1 | 531.5 | 229.6 |
| 1.262 | 861.0 | 1.0 | 5436.6 | 49.5 |
| 2.009 | 3725.8 | 198.3 | 1280.8 | 361.0 |
| 2.022 | 4115.1 | 209.4 | 501.1 | 6.9 |
| 2.025 | 966.4 | 103.5 | 498.8 | 74.2 |
| 2.026 | 2076.0 | 196.5 | 536.0 | 4.6 |
| 2.027 | 657.7 | 58.8 | 509.0 | 70.6 |
| 2.031 | 1357.9 | 0.6 | 326.4 | 52.7 |
| 2.038 | 2553.9 | 184.2 | 1397.0 | 345.6 |
| 2.039 | 1988.0 | 66.7 | 1010.3 | 195.5 |
| 2.041 | 3443.4 | 187.8 | 2095.1 | 161.9 |
| 2.046 | 1975.4 | 142.9 | 758.9 | 401.2 |
| 2.047 | 1942.1 | 163.1 | 437.5 | 184.9 |
| 2.054 | 414.8 | 5.7 | 438.9 | 207.3 |
| 2.055 | 977.5 | 72.3 | 311.6 | 180.9 |
| 2.058 | 1936.0 | 136.7 | 212.6 | 44.7 |
| 2.059 | 119.8 | 24.5 | 207.9 | 173.8 |
| 2.060 | 328.8 | 10.3 | 181.3 | 102.7 |
| 2.064 | 382.0 | 6.7 | 178.2 | 103.4 |
| 2.066 | 2510.4 | 30.5 | 368.3 | 133.1 |

### Example 16

### Kinase Panel Screen

### Relevance:

This assay demonstrates a compound's ability to inhibit members of a panel of kinases known to be involved in signaling pathways connected to inflammatory processes.

### Protocol

Compounds of Formulae I and II were examined for activity against a selected panel of kinases using the KinaseProfiler™ enzyme profiling services (Upstate, Millipore Bioscience Division). Percent kinase activity at 10 μM and 1 μM test compound and 10 μM ATP was determined against 40 wild-type recombinant human kinases according to Upstate's standard protocol: ASK1, BTK, CSK, c-RAF, GCK, GSK3β, IKKα, IKKβ, IRAK1, IRAK4, JNK1α1, JNK2α2, JNK3, ERK1, ERK2, MAPKAP-K2, MAPKAP-K3, MEK1, MKK4, MKK6, MKK7β, Mnk2, MSK1, PAK3, PDK1, PRAK, ROCK1, Rsk2, SAPK2a, SAPK2b, SAPK3, SAPK4, SRPK1, SRPK2, Syk, TAK1, TBK1, PI3-Kβ, PI3-Kγ, PI3-Kδ.

### Results:

Percent inhibition results are reported in Table 9 for four compounds against six kinases in the panel. Only compounds in which R₂ is R₂-2 were found to inhibit significantly GCK, ERK1/2, Mnk2 and IRAK1/2. Only ERK1/2 were inhibited by ˜50% at 1 μM by both compounds 2.059 and 2.066,

TABLE 9
Percent inhibition data for six of the tested kinases

| | | | | | | | Compound | |
|---|---|---|---|---|---|---|---|---|
| | Compound | | Compound | | Compound | | 1.162 | |
| | 2.059 | | 2.066 | | 1.161 | | | 10 |
| | 1 μM | 10 μM | 1 μM | 10 μM | 1 μM | 10 μM | 1 μM | μM |
| ERK1 | 37 | 4 | 52 | 15 | 97 | 75 | 84 | 50 |
| ERK2 | 56 | 12 | 50 | 12 | 104 | 92 | 89 | 60 |
| Mnk2 | 49 | 12 | 99 | 54 | 108 | 106 | 111 | 65 |
| IRAK4 | 63 | 22 | 77 | 25 | 96 | 109 | 105 | 88 |
| IRAK1 | 87 | 30 | 74 | 32 | 106 | 99 | 100 | 97 |
| GCK | 75 | 34 | 39 | 7 | 96 | 91 | 93 | 75 |

### Example 17

### Rodent Pharmacokinetic Analyses of ROCK Inhibitors

Plasma (EDTA K₂ anticoagulant) was collected from male, cannulated, CD Sprague Dawley rats to determine the pharmacokinetics of formulations containing compound inhibitors of Rho kinase. Each animal was dosed orally with a 4 ml/kg solution or suspension of each test compound in 10 mM acetate buffered saline, pH 4.5 at a final concentration range of 20-30 mol/kg. Blood was collected at 0.25, 0.5, 1, 2, 4, 6, 8, and 24 hours. Plasma samples were assayed for the concentration of the test compound using an on-line, solid phase extraction LC/MS/MS analysis system.

Samples were analyzed on a QSTAR Elite, hybrid quadrupole time-of-flight mass spectrometer (Applied Biosystems, Framingham, Mass.) coupled with a Symbiosis Pharma integrated, on-line SPE-HPLC system (Spark Holland Inc., Plainsboro, N.J.). Analyst QS 2.0 software was used for instrument control, data acquisition and processing. An aliquot of each sample was injected onto a Luna C18 column (50×2 mm, 4 um, 80 A, Phenomenex, Torrance, Calif.), and elution was carried out using a gradient from 2-98% acetonitrile. Mobile Phase A consisted of 0.1% ammonium hydroxide in water and Mobile Phase B consisted of 0.1% formic acid in acetonitrile. Pharmacokinetic analyses were performed using WinNonlin software version 5.2 (Pharsight Corporation, Mountain View, Calif.).

The pharmacokinetic results based on the observed plasma concentrations of the test compounds in rats are shown in Table 10.

TABLE 10
Pharmacokinetic results from rat oral PK studies
(mean plasma values for n = 3 rats)

| | Tmax | Cmax | AUC (0-last) | t½ | Vz_F |
|---|---|---|---|---|---|
| Compound | (hr) | (nM) | (nM * hr) | (hr) | (L/kg) |
| 1.131 | 0.83 | 5610 | 10825 | 1.55 | 6.8 |
| 1.092 | 0.25 | 2101 | 1849 | 1.74 | 19.0 |
| 1.123 | 0.33 | 2044 | 2064 | 0.9 | 14.8 |
| 2.038 | 0.5 | 1037 | 1283 | 0.71 | 22.5 |
| 2.039 | 0.33 | 783 | 905 | 1.13 | 59.4 |
| 1.074 | 0.42 | 735 | 1167 | 0.86 | 45.7 |
| 1.107 | 1.67 | 544 | 1586 | 1.28 | 36.3 |
| 1.124 | 0.5 | 415 | 535 | 1.39 | 93.4 |
| 2.045 | 0.67 | 223 | 456 | 1.59 | 226 |
| 1.108 | 0.83 | 209 | 415 | 1.36 | 116 |
| 1.091 | BLQ | BLQ | BLQ | BLQ | BLQ |
| 2.026 | BLQ | BLQ | BLQ | BLQ | BLQ |
| 1.136 | BLQ | BLQ | BLQ | BLQ | BLQ |

BLQ indicates that the compound was below the limit of quantitation in the assay

As determined from the plasma concentration versus time curves, the time to peak and peak exposure are represented by the values Tmax and Cmax, respectively. The AUC values (nM*hr) shown were calculated as the areas under the plasma concentration versus time curves from time zero through the time of the last observable value and represent the total exposure of the compound over the course of the study. Half-life values or the amount of time required for the plasma levels of the compound to decline to half the initial value are represented as t1/2. The volume of distribution (Vz_F expressed in L/kg) relates the amount of theoretical volume needed to account for the observed concentration of a given dose of a compound. For rats, the total body water content is approximately 0.15 L/kg. Calculated volumes of distribution below 0.15 L/kg are considered low, whereas values between 5 and 100 L/kg are considered high. The volume of distribution varies depending on the degree of plasma protein binding as well as partitioning of the compound into fat and tissues. Table 10 provides evidence that our ROCK inhibiting compounds have a varying degree of pharmacokinetic properties that would allow them to be optimized for multiple routes of administration. These compounds are quickly absorbed, as indicated by a Tmax of generally less than 1 hour, with varying degrees of peak and total exposure as indicated by Cmax and AUC, with higher values indicating greater exposure. Regardless of exposure, these compounds demonstrate a similar clearance, t1/2.

Additionally, compound concentrations were determined in the plasma and lungs of male, ovalbumin-sensitized, Balb/c mice from a murine model of asthma. Test compounds were formulated in water or 1% polysorbate 80 and dosed at 15 μmol/kg for intraperitoneal (IP) or oral (PO) administration or formulated for intratracheal (IT) administration and dosed at 5 μmol/kg, which directly targets the lungs. Following completion of the in vivo study, mice were euthanized and blood and plasma collected approximately 2.5-3 hours post administration of test compound for bronchodialator (BD) studies and 24 hours post administration for anti-inflamatory (AI) studies. Lungs were homogenized in Matrix A lysing tubes using a FastPrep 24 tissue and cell homogenizer (MP Biomedicals, Solon, Ohio). Both plasma samples and lung extracts were assayed for compound concentrations using an on-line, solid phase extraction LC/MS/MS system. The actual lung tissue concentrations of each compound in mouse were extrapolated from the lung and plasma concentrations, data are shown in Table 11. The results of a set of experiments using unsensitized mice and collecting only plasma 15 minutes post administration of test compounds are shown in Table 12.

TABLE 11
Compound concentrations in ova-sensitized, ova-challenged
mice lungs post IP, PO and IT administration (mean plasma
corrected lung values for n = 9 or 10 mice)

| Compound | Efficacy Model | Route | Time Point, h | Lung, nM¹ |
|---|---|---|---|---|
| 1.131 | BD | PO | 3 | 7353 |
| 2.038 | BD | PO | 3 | 440 |
| 1.092 | BD | PO | 3 | 152 |
| 1.091 | BD | IP | 3 | 117 |
| 1.091 | BD | IT | 2.5 | 123 |
| 1.131 | AI | PO | 24 | 33 |
| 2.038 | AI | PO | 24 | 11 |

¹for calculation of lung concentrations, it was assumed that 22.6% of the lung mass was plasma (R. H. Storey, Cancer Research, 943-947, 1951)

TABLE 12
Compound concentrations in mice at 15 min post administration
(mean plasma values for n = 3 mice)

| | | Plasma |
|---|---|---|
| | Plasma Mean | Concentration |
| Compound | Concentration, nM | StdDev, nM |
| 1.072 | 1770.9 | 320.9 |
| 1.074 | 506.1 | 407.9 |
| 1.075 | 348.0 | 83.9 |
| 1.076 | 1715.0 | 474.9 |
| 1.077 | 25.9 | 0.2 |
| 1.078 | 1018.8 | 75.8 |
| 1.079 | 2442.5 | 302.9 |
| 1.090 | 5.9 | 5.2 |
| 1.091 | 333.8 | 82.7 |
| 1.092 | 314.3 | 60.4 |
| 1.093 | 362.6 | 148.7 |
| 1.106 | 441.4 | 146.7 |
| 1.107 | 211.1 | 129.5 |
| 1.108 | 394.5 | 9.0 |
| 1.109 | 187.2 | 36.0 |
| 1.110 | 792.0 | 311.9 |
| 1.123 | 71.4 | 11.8 |
| 1.124 | 118.0 | 2.4 |
| 1.126 | 0.0 | 0.0 |
| 1.127 | 980.2 | 757.5 |
| 1.131 | 444.5 | 130.0 |
| 1.132 | 982.4 | 207.7 |
| 1.133 | 1097.9 | 234.3 |
| 1.134 | 1550.8 | 623.9 |
| 1.135 | 656.8 | 115.4 |
| 1.136 | 25.9 | 6.3 |
| 1.137 | 556.9 | 279.8 |
| 1.138 | 1863.8 | 378.7 |
| 1.141 | 1643.1 | 368.6 |
| 1.142 | 329.7 | 171.6 |
| 1.143 | 274.5 | 68.8 |
| 1.145 | 109.0 | 117.9 |
| 1.146 | 1255.7 | 703.5 |
| 1.148 | 767.1 | 63.9 |
| 1.149 | 1559.4 | 789.6 |
| 1.150 | 1392.3 | 1278.3 |
| 1.151 | 478.6 | 173.6 |
| 1.152 | 435.4 | 44.5 |
| 1.153 | 521.5 | 61.3 |
| 1.154 | 1039.5 | 447.9 |
| 1.155 | 32.4 | 36.3 |
| 1.156 | 88.0 | 37.5 |
| 1.157 | 357.2 | 131.9 |
| 1.158 | 101.6 | 54.4 |
| 1.159 | 250.5 | 343.2 |
| 1.161 | 392.5 | 14.9 |
| 1.162 | 76.1 | 12.9 |
| 1.163 | 10.1 | 1.1 |
| 1.164 | 1504.3 | 580.6 |
| 1.165 | 93.5 | 49.6 |
| 1.166 | 342.4 | 118.1 |
| 1.168 | 587.5 | 258.9 |
| 1.170 | 638.6 | 154.7 |
| 1.171 | 368.8 | 208.9 |
| 1.172 | 111.1 | 32.0 |
| 1.173 | 144.4 | 72.6 |
| 1.175 | 1126.5 | 112.5 |
| 1.176 | 89.1 | 69.1 |
| 1.177 | 283.1 | 125.6 |
| 1.182 | 452.5 | 297.7 |
| 1.183 | 708.5 | 359.6 |
| 1.185 | 1023.6 | 492.8 |
| 1.186 | 2169.4 | 1599.1 |
| 1.191 | 260.0 | 58.8 |
| 1.193 | 55.4 | 26.0 |
| 1.194 | 355.0 | 133.5 |
| 1.195 | 107.9 | 23.1 |
| 1.197 | 453.1 | 354.0 |
| 1.198 | 643.2 | 112.1 |
| 1.200 | 0.0 | 0.0 |
| 1.202 | 129.7 | 71.9 |
| 1.203 | 1134.7 | 44.2 |
| 1.204 | 549.1 | 183.6 |
| 1.206 | 671.5 | 80.9 |
| 1.208 | 281.1 | 45.4 |
| 1.210 | 285.8 | 122.9 |
| 1.212 | 863.4 | 104.1 |
| 1.213 | 396.4 | 135.1 |
| 1.215 | 2651.2 | 529.0 |
| 1.217 | 292.5 | 176.0 |
| 1.219 | 1678.9 | 516.3 |
| 1.223 | 12.8 | 0.6 |
| 1.226 | 526.1 | 157.9 |
| 1.227 | 1859.4 | 603.7 |
| 1.229 | 1453.9 | 465.0 |
| 1.233 | 41.1 | 11.6 |
| 1.234 | 239.6 | 79.4 |
| 1.236 | 47.7 | 18.1 |
| 1.237 | 178.4 | 64.6 |
| 1.238 | 48.3 | 29.6 |
| 1.239 | 258.9 | 111.8 |
| 1.241 | 991.4 | 134.5 |
| 1.242 | 579.8 | 314.0 |
| 1.245 | 1524.0 | 127.5 |
| 1.246 | 587.4 | 299.7 |
| 1.249 | 2147.1 | 688.2 |
| 1.252 | 1259.2 | 1210.0 |
| 1.253 | 240.0 | 20.3 |
| 1.258 | 567.5 | 223.5 |
| 1.259 | 264.4 | 39.1 |
| 1.260 | 291.2 | 120.7 |
| 1.262 | 285.2 | 76.2 |
| 2.025 | 73.7 | 21.2 |
| 2.026 | 629.5 | 94.6 |
| 2.027 | 502.6 | 248.5 |
| 2.031 | 1430.4 | 139.2 |
| 2.034 | 664.7 | 649.4 |
| 2.036 | 1343.9 | 1603.3 |
| 2.038 | 728.9 | 222.8 |
| 2.039 | 92.0 | 47.6 |
| 2.041 | 986.5 | 287.0 |
| 2.043 | 60.8 | 24.7 |
| 2.046 | 488.1 | 96.1 |
| 2.047 | 3.0 | 1.7 |
| 2.054 | 765.5 | 214.3 |
| 2.055 | 656.1 | 172.6 |
| 2.056 | 1257.0 | 230.6 |
| 2.057 | 431.2 | 41.5 |
| 2.058 | 193.6 | 167.4 |
| 2.059 | 89.6 | 21.5 |
| 2.060 | 307.6 | 157.6 |
| 2.061 | 73.2 | 21.1 |
| 2.062 | 659.9 | 582.8 |
| 2.063 | 347.9 | 248.5 |
| 2.064 | 201.6 | 78.7 |
| 2.065 | 236.4 | 29.8 |
| 2.066 | 491.6 | |

The results of these quantitative analyses have enabled the selection of compounds for additional studies based on desirable pharmacokinetic profiles and preferential distribution in the target organ (lungs). We have identified compounds which possess high bioavailability and efficacy against airway hyperreactivity when dosed orally, as well as compounds that are efficacious when administered intraperitoneally or intratracheally, but do not reach systemic levels when dosed orally and thus are not efficacious by the oral route. Characterization of the pharmacokinetic properties and distribution of these Rho Kinase inhibitors is an essential part of the selection of compounds for drug development.

### Example 18

### Efficacy of Compounds of Formula I or II to Inhibit Proliferation of Primary Smooth-Muscle Like Cells Derived from Human LAM Patients

### Relevance

This assay measures the ability of a compound to directly inhibit the proliferation of primary smooth-muscle like cells derived from human LAM patients. Activity of compounds in this assay supports the use of these compounds for diseases with a proliferative component.

### Protocol

LAM cells were dissociated from LAM nodules from the lung of patients with LAM who have undergone lung transplant. In brief, cells were dissociated by enzymatic digestion in M199 medium containing 0.2 mM CaCl₂, 2 mg/ml collagenase D, 1 mg/ml trypsin inhibitor, and 3 mg/ml elastase. The cell suspension was filtered and then washed with equal volumes of cold DF8 medium, consisting of equal amounts of Ham's F-12 and Dulbecco's modified Eagle's medium supplemented with 1.6×10⁻⁶ M ferrous sulfate, 1.2×10⁻⁵ U/ml vasopressin, 1.0×10⁻⁹ M triiodothyronine, 0.025 mg/ml insulin, 1.0×10⁻⁸ M cholesterol, 2.0×10⁻⁷ M hydrocortisone, 10 pg/ml transferrin, and 10% fetal bovine serum. The cells were cultured in DF8 medium and were passaged twice per week. All LAM cells had a high degree of proliferative activity in the absence of any stimuli. Two separate LAM cell lines were tested and denoted as LAM1 or LAM2 cells. LAM cells in subculture during the 3rd through 12th cell passages were used. DNA synthesis was measured using a [3H]thymidine incorporation assay. In brief, near-confluent cells that were serum-deprived for 48 h were incubated with 10 μM of compound or with vehicle (control). After 18 h of incubation, cells were labeled with [methyl-3H]thymidine for 24 hours. The cells were then scraped and lysed, and DNA was precipitated with 10% trichloroacetic acid. The precipitants were aspirated on glass filters and extensively washed and dried, and [³H]thymidine incorporation was counted (Goncharova et al., Mol Pharmacol 73:778-788, 2008)

### Results

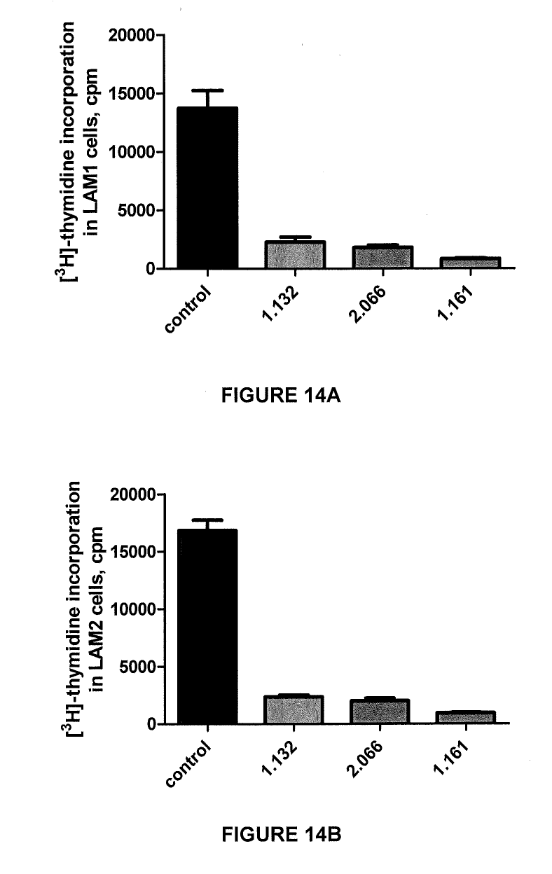

As shown in FIGS. 14A and 14B, compounds of Formula I and II reduced proliferation of LAM1 (FIG. 14A) and LAM2 (FIG. 14B) cells when dosed in vitro at 10 μM. These results demonstrate that Compounds of Formula I and II are efficacious in inhibiting the proliferation of primary cells.

### Example 19

### Summary of Data of Preferred Compounds

Principal biological data describing the preferred compounds of the invention have been collected into Table 13. Displayed in this table are ROCK1 and ROCK2 average Ki values in nM (as detailed in Example 1), Akt3 and p70S6K average Ki values in nM (as detailed in Example 15), average percent of PDGF stimulated proliferation at 10 and 1 μM of test compound (as detailed in Example 14), average percent of stimulated IL-1β, IL-6, and TNF-α secretion from human monocytes at 10 μM of test compound (as detailed in Example 12), average IC₅₀ for inhibition of fMLP-induced neutrophil chemotaxis in μM (as detailed in Example 3), mean compound plasma concentrations in mice at 15 minutes post oral administration (as detailed in Example 17).

TABLE 13
Summary of Data of Preferred Compounds

| | ROCK1 | ROCK2 | Akt3 Ki, | p70S6K | Proliferation | Proliferation | | | | Chemotaxis IC50, | Mouse |
|---|---|---|---|---|---|---|---|---|---|---|---|
| Compound | Ki, nM | Ki, nM | nM | Ki, nM | at 10 μM, % | at 1 μM, % | IL-1β % | IL-6, % | TNF-α % | μM | Oral PK, nM |
| 1.074 | 40.1 | 4.1 | 437.4 | 548.3 | 46.9 | 79.9 | 43.9 | 96.0 | 87.7 | | 506 |
| 1.075 | 48.7 | 4.4 | 5321.5 | 974.6 | | | 49.7 | 73.9 | 51.6 | | 348 |
| 1.076 | 14.3 | 2.6 | 240.9 | 414.3 | 53.7 | 84.0 | 51.0 | 81.2 | 78.9 | | 1715 |
| 1.077 | 76.1 | 11.1 | 5253.2 | 715.5 | | | 30.3 | 43.3 | 52.3 | | 26 |
| 1.079 | 71.5 | 4.7 | 7191.7 | 3012.8 | | | 59.3 | 31.1 | 56.5 | | 2443 |
| 1.091 | 71.4 | 3.3 | 5388.5 | 1420.4 | 69.3 | 85.7 | 165.5 | 108.2 | 104.6 | 2.3 | 334 |
| 1.093 | 64.5 | 7.7 | 1824.9 | 2025.6 | | 109.0 | 49.7 | 76.1 | | 363 | |
| 1.108 | 25.6 | 6.5 | 205.0 | 510.6 | 43.7 | 83.1 | 131.3 | 89.8 | 116.7 | | 395 |
| 1.109 | 58.8 | 9.6 | 5190.9 | 2495.5 | | | 190.5 | 312.9 | 118.3 | | 187 |
| 1.123 | 82.3 | 9.6 | 2406.9 | 2810.7 | | | 82.6 | 64.7 | 62.7 | 3.1 | 71 |
| 1.124 | 64.5 | 3.3 | 7868.0 | 3325.3 | 61.6 | 68.5 | 99.5 | 101.4 | 61.5 | 3.4 | 118 |
| 1.126 | 76.2 | 17.2 | | | | | | | | | 0 |
| 1.131 | 19.7 | 3.8 | 282.6 | 502.8 | 36.6 | 61.7 | 48.3 | 68.6 | 85.2 | 1.6 | 445 |
| 1.132 | 22.5 | 3.5 | 81.8 | 514.6 | 30.3 | 48.9 | 58.6 | 72.5 | 80.3 | | 982 |
| 1.133 | 25.0 | 4.3 | 148.3 | 531.8 | | | 54.5 | 70.7 | 66.2 | | 1098 |
| 1.134 | 22.4 | 4.4 | 150.7 | 519.7 | | | 43.2 | 74.6 | 69.1 | | 1551 |
| 1.135 | 40.3 | 5.4 | 444.2 | 588.6 | 35.0 | 52.6 | 57.0 | 123.2 | 108.0 | | 657 |
| 1.136 | 25.8 | 5.1 | 289.7 | 1236.7 | 39.8 | 71.4 | 66.3 | 95.0 | 71.5 | 2.6 | 26 |
| 1.137 | 36.3 | 7.2 | 197.9 | 353.6 | | | 40.3 | 46.2 | 58.0 | | 557 |
| 1.138 | 41.1 | 6.3 | 91.3 | 443.5 | 27.0 | 46.3 | 257.4 | 76.6 | 130.9 | 1.9 | 1864 |
| 1.141 | 28.5 | 3.8 | 1263.0 | 387.5 | | | 50.4 | 71.7 | 75.7 | | 1643 |
| 1.148 | 24.3 | 3.6 | 1131.4 | 435.5 | 63.5 | 56.9 | 63.9 | 78.6 | 56.1 | | 767 |
| 1.149 | 46.8 | 4.2 | 7395.9 | 1888.4 | | | 69.8 | 121.5 | 119.9 | | 1559 |
| 1.150 | 33.2 | 3.2 | 3183.1 | 1273.8 | | | 78.2 | 89.2 | 94.4 | | 1392 |
| 1.152 | 19.8 | 3.3 | 1976.2 | 523.5 | | | 74.7 | 94.7 | 120.1 | | 435 |
| 1.153 | 62.8 | 4.2 | 9950.2 | 2376.1 | | | 64.1 | 106.2 | 74.3 | | 522 |
| 1.155 | 45.4 | 7.0 | 5680.5 | 1751.6 | | | 76.7 | 121.8 | 79.7 | | 32 |
| 1.156 | 135.8 | 13.0 | 8772.6 | 3244.6 | | | 60.7 | 92.5 | 70.5 | | 88 |
| 1.157 | 263.8 | 8.8 | 29192.3 | 8693.4 | | | 121.4 | 92.6 | 65.1 | | 357 |
| 1.158 | 64.1 | 5.1 | 5905.2 | 1971.7 | | | 80.8 | 133.1 | 86.6 | | 102 |
| 1.161 | 9.9 | 2.5 | 63.5 | 129.4 | 33.4 | 50.0 | 87.7 | 86.3 | 153.5 | | 392 |
| 1.162 | 15.2 | 2.8 | 92.0 | 387.4 | 42.5 | 55.6 | 95.5 | 99.8 | 158.7 | | 76 |
| 1.163 | 33.6 | 2.9 | 4423.8 | 1875.2 | | | 166.7 | 140.9 | 91.6 | | 10 |
| 1.164 | 42.4 | 6.1 | 4306.8 | 1957.4 | | | 80.1 | 109.5 | 89.0 | | 1504 |
| 1.165 | 50.7 | 3.4 | 4140.0 | 1627.1 | 57.9 | 74.8 | 129.9 | 114.3 | 103.5 | | 94 |
| 1.166 | 95.2 | 8.0 | 18132.9 | 5163.5 | | | 107.0 | 87.2 | 82.2 | | 342 |
| 1.171 | 109.2 | 16.0 | 9326.9 | 3419.0 | | | 78.9 | 91.8 | 72.2 | | 369 |
| 1.173 | 15.1 | 3.6 | 157.0 | 339.7 | 35.8 | 55.4 | 86.1 | 79.5 | 80.1 | | 144 |
| 1.175 | 65.9 | 7.6 | 2820.2 | 853.0 | 49.0 | 58.2 | 29.3 | 38.2 | 47.4 | | 1126 |
| 1.176 | 314.3 | 11.2 | 20941.5 | 8755.7 | | | 95.2 | 112.4 | 72.4 | | 89 |
| 1.186 | 129.3 | 11.9 | 10237.7 | 1612.5 | | | 64.1 | 105.3 | 68.2 | | 2169 |
| 1.193 | 64.9 | 14.8 | | | | | | | | | 55 |
| 1.195 | 196.2 | 10.3 | 21975.8 | 2731.0 | | | 115.4 | 94.4 | 67.7 | | 108 |
| 1.197 | 120.2 | 5.0 | 64051.2 | 8688.8 | 48.9 | 52.5 | 179.1 | 128.8 | 83.3 | | 453 |
| 1.200 | 76.5 | 5.9 | 10608.5 | 3903.1 | | | 0.0 | 0.0 | 0.2 | | 0 |
| 1.206 | 64.4 | 9.1 | 529.1 | 314.4 | 51.1 | 77.5 | 88.7 | 164.0 | 97.3 | | 672 |
| 1.212 | 44.2 | 3.9 | 390.2 | 894.0 | | | 116.3 | 111.0 | 108.1 | | 863 |
| 1.213 | 106.3 | 3.0 | 3207.8 | 2097.2 | 52.3 | 70.1 | 111.1 | 81.7 | 77.4 | | 396 |
| 1.215 | 102.8 | 3.5 | 4753.0 | 1285.8 | 54.0 | 70.8 | 136.7 | 63.2 | 60.4 | | 2651 |
| 1.217 | 70.1 | 12.1 | 10301.1 | 3501.9 | | | 118.6 | 73.8 | 71.3 | | 293 |
| 1.219 | 343.6 | 15.4 | 38297.7 | 4969.9 | | | 138.9 | 127.7 | 82.1 | | 1679 |
| 1.223 | 239.5 | 15.7 | 11139.0 | 3101.9 | | | 117.0 | 88.5 | 60.7 | | 13 |
| 1.233 | 47.2 | 1.3 | 2628.6 | 2004.9 | | | 78.5 | 78.9 | 79.0 | | 41 |
| 1.236 | 49.3 | 2.1 | 3716.5 | 2755.4 | | | 75.2 | 93.0 | 98.0 | | 48 |
| 1.237 | 286.7 | 4.0 | 7910.2 | 9873.2 | 51.4 | 63.5 | 97.1 | 100.9 | 70.6 | | 178 |
| 1.238 | 61.2 | 1.5 | 4171.1 | 2609.6 | 48.6 | 40.7 | 101.1 | 62.9 | 73.2 | | 48 |
| 1.239 | 282.6 | 6.3 | 17657.7 | 10026.9 | 37.8 | 41.7 | 39.4 | 84.7 | 58.5 | | 259 |
| 1.249 | 91.7 | 8.6 | 1599.7 | 937.5 | | | 133.8 | 56.2 | 60.0 | | 2147 |
| 1.252 | 30.5 | 4.5 | 205.0 | 170.7 | | | 139.2 | 68.3 | 101.6 | | 1259 |
| 1.253 | 59.9 | 1.7 | 2597.1 | 2515.0 | 47.9 | 44.8 | 160.6 | 228.6 | 126.8 | | 240 |
| 1.258 | 9.5 | 1.3 | 315.2 | 531.5 | 43.4 | 50.5 | 104.1 | 83.5 | 94.0 | | 567 |
| 1.259 | 19.5 | 2.1 | | | | | | | | | 264 |
| 1.260 | 70.9 | 7.1 | | | | | | | | | 291 |
| 1.261 | 307.4 | 14.8 | | | | | | | | | |
| 1.262 | 54.9 | 4.0 | 861.0 | 5436.6 | | | 145.7 | 156.6 | 135.3 | | 285 |
| 1.270 | 130.5 | 9.9 | | | | | | | | | |
| 1.273 | 31.3 | 8.2 | | | | | | | | | |
| 1.275 | 401.7 | 14.1 | | | | | | | | | |
| 1.277 | 42.3 | 4.6 | | | | | | | | | |
| 1.281 | 71.8 | 7.4 | | | | | | | | | |
| 2.025 | 6.9 | 2.9 | 966.4 | 498.8 | 68.0 | 69.8 | | | | 1.7 | 74 |
| 2.026 | 38.0 | 13.0 | 2076.0 | 536.0 | 52.0 | 74.5 | 166.0 | 180.7 | 109.1 | 3.8 | 629 |
| 2.031 | 14.6 | 5.3 | 1357.9 | 326.4 | 52.6 | 90.3 | 49.0 | 89.3 | 66.4 | | 1430 |
| 2.038 | 28.9 | 6.3 | 2553.9 | 1397.0 | 62.7 | 58.6 | 90.8 | 79.7 | 70.2 | 0.7 | 729 |
| 2.039 | 18.8 | 6.7 | 1988.0 | 1010.3 | | | 49.8 | 70.3 | 47.8 | 1.6 | 92 |
| 2.041 | 30.8 | 9.6 | 3443.4 | 2095.1 | 61.5 | 81.8 | | | | | 987 |
| 2.046 | 16.7 | 5.6 | 1975.4 | 758.9 | 32.1 | 57.4 | | | | | 488 |
| 2.047 | 26.4 | 7.0 | 1942.1 | 437.5 | 53.8 | 65.3 | | | | | 3 |
| 2.054 | 17.1 | 3.7 | 414.8 | 438.9 | 84.6 | 68.2 | 24.0 | 56.8 | 37.9 | | 765 |
| 2.055 | 16.0 | 6.4 | 977.5 | 311.6 | | | | | | | 656 |
| 2.057 | 6.2 | 3.7 | | | | | | | | | 431 |
| 2.058 | 15.3 | 3.3 | 1936.0 | 212.6 | | | 1.2 | 1.3 | 10.6 | | 194 |
| 2.059 | 3.9 | 2.7 | 119.8 | 207.9 | 25.5 | 75.0 | 0.3 | 0.0 | 6.9 | | 90 |
| 2.060 | 4.9 | 3.2 | 328.8 | 181.3 | | | 5.9 | 19.6 | 33.0 | | 308 |
| 2.061 | 10.5 | 1.8 | | | | | | | | | 73 |
| 2.064 | 4.1 | 2.2 | 382.0 | 178.2 | 56.2 | 53.1 | 14.3 | 45.7 | 66.2 | | 202 |
| 2.065 | 4.1 | 1.8 | | | | | | | | | 236 |
| 2.066 | 10.2 | 2.3 | 2510.4 | 368.3 | 19.8 | 20.0 | 0.0 | 0.0 | 25.2 | | 492 |
| 2.067 | 19.6 | 4.2 | | | | | | | | | |
| 2.068 | 8.0 | 5.8 | | | | | | | | | |
| 2.069 | 16.7 | 2.4 | | | | | | | | | |
| 2.072 | 7.5 | 4.4 | | | | | | | | | |
| 2.073 | 12.7 | 4.2 | | | | | | | | | |
| 2.076 | 8.0 | 2.4 | | | | | | | | | |
| 2.077 | 33.7 | 5.0 | | | | | | | | | |
| 2.078 | 18.3 | 2.6 | | | | | | | | | |
| 2.079 | 18.5 | 2.3 | | | | | | | | | |
| 2.082 | 131.7 | 9.0 | | | | | | | | | |
| 2.096 | 70.2 | 9.6 | | | | | | | | | |
| 2.097 | 35.4 | 2.8 | | | | | | | | | |
| 2.099 | 15.0 | 3.8 | | | | | | | | | |

Although the invention has been described with reference to the presently preferred embodiments, it should be understood that various modifications could be made without departing from the scope of the invention.

What is claimed:

1. A method of treating rheumatoid arthritis or inflammatory bowel disease, comprising the steps of first identifying a subject suffering from rheumatoid arthritis or inflammatory bowel disease, then administering to the subject an effective amount of a compound of Formula II to treat rheumatoid arthritis or inflammatory bowel disease; 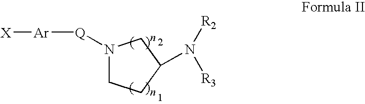
wherein:
Q is C═O, SO₂, or (CR₄R₅)ₙ₃;
n₁ is 1, 2, or 3;
n₂ is 1 or 2;
n₃ is 0, 1, 2, or 3;
wherein the ring represented by 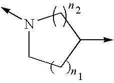
is optionally substituted by alkyl, halo, oxo, OR₆, NR₆R₇, or SR₆;
R₂ is R₂-1 or R₂-2, optionally substituted: 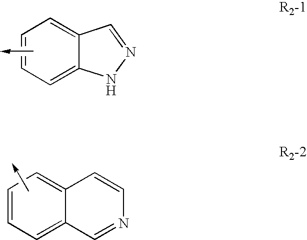
Ar is a monocyclic or bicyclic aryl or heteroaryl ring;
X is from 1 to 3 substituents on Ar, and each is independently selected from the group consisting of OR₈, NR₈R₉, SR₈, SOR₈, SO₂R₈, SO₂NR₈R₉, NR₈SO₂R₉, CONR₈R₉, NR₈C(═O)R₉, NR₈C(═O)OR₉, OC(═O)NR₈R₉, and NR₈C(═O)NR₉R₁₀,
R₃-R₇ are independently H, alkyl, alkenyl, alkynyl, cycloalkyl, cycloalkenyl, cycloalkylalkyl, cycloalkylalkenyl, or cycloalkylalkynyl, optionally substituted;
R₈ is H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle; optionally substituted by one or more halogen or heteroatom-containing substituents selected from the group consisting of OR₁₁, NR₁₁R₁₂, NO₂, SR₁₁, SOR₁₁, SO₂R₁₁, SO₂NR₁₁R₁₂, NR₁₁SO₂R₁₂, OCF₃, CONR₁₁R₁₂, NR₁₁C(═O)R₁₂, NR₁₁C(═O)OR₁₂, OC(═O)NR₁₁R₁₂, and NR₁₁C(═O)NR₁₂R₁₃;
R₉ and R₁₀ are independently H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle; optionally substituted by one or more halogen or heteroatom-containing substituents selected from the group consisting of OR₁₄, NR₁₄R₁₅, NO₂, SR₁₄, SOR₁₄, SO₂R₁₄, SO₂NR₁₄R₁₅, NR₁₄SO₂R₁₅, OCF₃, CONR₁₄R₁₅, NR₁₄C(═O)R₁₅ NR₁₄C(═O)OR₁₅, OC(═O)NR₁₄R₁₅, and NR₁₄C(═O)NR₁₅R₁₆;
wherein any two of the groups R₈, R₉ and R₁₀ are optionally joined with a link selected from the group consisting of bond, —O—, —S—, —SO—, —SO₂—, and —NR₁₇— to form a ring;
R₁₁-R₁₇ are independently H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle;
with the first proviso that if X is acyclic and is connected to Ar by a carbon atom, then X contains at least one nitrogen or sulfur atom,
with the second proviso that if X is acyclic and is connected to Ar by an oxygen or nitrogen atom, then X contains at least one additional oxygen, nitrogen or sulfur atom, and with the third proviso that if X is connected to Ar by a —SO₂— linkage, then R₂ is not nitrogen- or oxygen-substituted R₂-2.

2. The method according to claim 1, wherein said compound of Formula II is a compound of Formula Ia, IIb, or IIc: 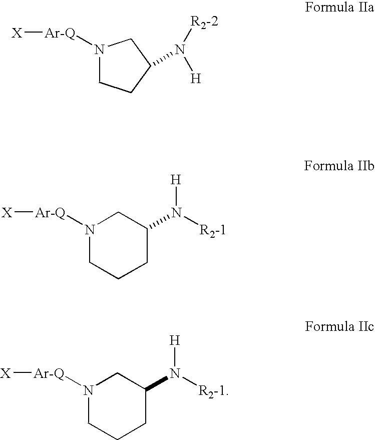
wherein Ar is phenyl, a 6,5-fused bicyclic heteroaryl ring, or a 6,6-fused bicyclic heteroaryl ring; Ar is substituted by 1 or 2 substituents X, and Q is CH₂.

3. The method according to claim 2, wherein Ar is 3-substituted phenyl; 4-substituted phenyl; 3,4-disubstituted phenyl; or 2,3-disubstituted phenyl.

4. The method according to claim 2, wherein Ar is benzofuran, benzothiophene, indole, and benzimidazole.

5. The method according to claim 1, wherein said compound is Compound 1.074, which is (R)—N-(1-(4-(methylthio)benzyl)piperidin-3-yl)-1H-indazol-5-amine; Compound 1.075, which is (S)—N-(1-(4-(methylthio)benzyl)piperidin-3-yl)-1H-indazol-5-amine; Compound 1.091, which is (S)—N-(3-((3-(1H-indazol-5-ylamino)piperidin-1-yl)methyl)phenyl)methanesulfonamide; Compound 1.093, which is (R)—N-(3-((3-(1H-indazol-5-ylamino)piperidin-1-yl)methyl)phenyl)methanesulfonamide; Compound 1.123, which is (R)—N-(3-((3-(1H-indazol-5-ylamino)piperidin-1-yl)methyl)phenyl)ethanesulfonamide; Compound 1.124, which is (S)—N-(3-((3-(1H-indazol-5-ylamino)piperidin-1-yl)methyl)phenyl)ethanesulfonamide; Compound 1.126, which is (R)-2-(3-((3-(1H-indazol-5-ylamino)piperidin-1-yl)methyl)phenoxy)-N-(pyridin-3-yl)acetamide; Compound 1.152, which is (S)-2-(5-((3-(1H-indazol-5-ylamino)piperidin-1-yl)methyl)-2-methylphenoxy)ethanol; Compound 1.157, which is (S)—N-(1-(3-(methylsulfonylmethyl)benzyl)piperidin-3-yl)-1H-indazol-5-amine; Compound 1.158, which is (S)—N-(1-(3-(methylthio)benzyl)piperidin-3-yl)-1H-indazol-5-amine; Compound 1.161, which is (R)-2-(5-((3-(1H-indazol-5-ylamino)piperidin-1-yl)methyl)-2-methylphenoxy)ethanol; Compound 1.195, which is (S)-2-(3-((3-(1H-indazol-5-ylamino)piperidin-1-yl)methyl)phenoxy)acetamide; Compound 1.200, which is (S)-ethyl 2-(3-((3-(1H-indazol-5-ylamino)piperidin-1-yl)methyl)phenoxy)acetate; Compound 1.212, which is (R)—N-(5-((3-(1H-indazol-5-ylamino)piperidin-1-yl)methyl)-2-chlorophenyl)methanesulfonamide; Compound 1,213, which is (S)—N-(5-((3-(1H-indazol-5-ylamino)piperidin-1-yl)methyl)-2-chlorophenyl)methanesulfonamide; Compound 1.215, which is (S)-3-((3-(1H-indazol-5-ylamino)piperidin-1-yl)methyl)benzenesulfonamide; Compound 1.219, which is (S)-3-((3-(1H-indazol-5-ylamino)piperidin-1-yl)methyl)benzamide; Compound 1.233, which is (S)—N-(5-((3-(1H-indazol-5-ylamino)piperidin-1-yl)methyl)-2-methylphenyl)methanesulfonamide; Compound 1.236, which is (S)—N-(5-((3-(1H-indazol-5-ylamino)piperidin-1-yl)methyl)-2-methylphenyl)butane-1-sulfonamide; Compound 1.237, which is (S)—N-(2-((3-(1H-indazol-5-ylamino)piperidin-1-yl)methyl)-5-methylphenyl)-N′,N′dimethylaminosulfamide; Compound 1.238, which is (S)—N-(5-((3-(1H-indazol-5-ylamino)piperidin-1-yl)methyl)-2-methylphenyl)propane-1-sulfonamide; Compound 1.239, which is (S)—N-(5-((3-(1H-indazol-5-ylamino)piperidin-1-yl)methyl)-2-methylphenyl)-4-methylbenzenesulfonamide; Compound 1.249, which is (R)-3-((3-(1H-indazol-5-ylamino)piperidin-1-yl)methyl)benzenesulfonamide; Compound 1.253, which is (S)—N-(5-((3-(1H-indazol-5-ylamino)piperidin-1-yl)methyl)-2-methylphenyl)ethanesulfonamide; Compound 1.258, which is (R)—N-(5-((3-(1H-indazol-5-ylamino)piperidin-1-yl)methyl)-2-methylphenyl)methanesulfonamide; Compound 1.259, which is (R)—N-(5-((3-(1H-indazol-5-ylamino)piperidin-1-yl)methyl)-2-methylphenyl)ethanesulfonamide; Compound 1.260, which is (R)—N-(5-((3-(1H-indazol-5-ylamino)piperidin-1-yl)methyl)-2-methylphenyl)-4-methylbenzenesulfonamide; Compound 1.261, which is (S)—N-(3-((3-(1H-indazol-5-ylamino)piperidin-1-yl)methyl)phenyl)-N′N′dimethylaminosulfamide; Compound 1.262, which is (R)—N-(2-((3-(1H-indazol-5-ylamino)piperidin-1-yl)methyl)-5-methylphenyl)-N′,N′dimethylaminosulfamide; Compound 1.270, which is (S)—N-(3-((3-(1H-indazol-5-ylamino)piperidin-1-yl)methyl)phenyl)piperidine-1-sulfonamide; Compound 1.275, which is (S)—N-(3-((3-(1H-indazol-5-ylamino)piperidin-1-yl)methyl)-2-methylphenyl)-N′,N′ dimethylaminosulfamide; Compound 1.281, which is (R)-2-(5-((3-(1H-indazol-5-ylamino)piperidin-1-yl)methyl)-2-methylphenyl1H-indazol-5-ylamino)piperidin-1-yl)methyl)-2-methylphenoxy)acetamide; Compound 2.026, which is (R)—N-(1-(4-(methylthio)benzyl)pyrrolidin-3-yl)isoquinolin-5-amine; Compound 2.038, which is (R)—N-(3-((3-(isoquinolin-5-ylamino)pyrrolidin-1-yl)methyl)phenyl)methanesulfonamide; Compound 2.039, which is (R)-2-(3-((3-(isoquinolin-5-ylamino)pyrrolidin-1-yl)methyl)phenoxy)ethanol; Compound 2.041, which is (R)—N-(3-((3-(isoquinolin-5-ylamino)pyrrolidin-1-yl)methyl)phenyl)ethanesulfonamide; Compound 2.054, which is (R)—N-(5-((3-(isoquinolin-5-ylamino)pyrrolidin-1-yl)methyl)-2-methylphenyl)ethanesulfonamide; Compound 2.064, which is (R)-2-(5-((3-(isoquinolin-5-ylamino)pyrrolidin-1-yl)methyl)-2-methylphenoxy)ethanol; Compound 2.067, which is (R)-2-(5-((3-(isoquinolin-5-ylamino)pyrrolidin-1-yl)methyl)-2-methoxyphenoxy)ethanol; Compound 2.068, which is (R)-2-(2-fluoro-5-((3-(isoquinolin-5-ylamino)pyrrolidin-1-yl)methyl)phenoxy)ethanol; Compound 2.069, which is (R)—N-(3-((3-(isoquinolin-5-ylamino)pyrrolidin-1-yl)methyl)phenyl)piperidine-1-sulfonamide; Compound 2.073, which is (R)-2-(5-((3-(isoquinolin-5-ylamino)pyrrolidin-1-yl)methyl)-2-methylphenoxy)acetic acid; Compound 2.076, which is (R)—N-(5-((3-(isoquinolin-5-ylamino)pyrrolidin-1-yl)methyl)-2-methylphenyl)methanesulfonamide; Compound 2.077, which is (R)—N-(5-((3-(isoquinolin-5-ylamino)pyrrolidin-1-yl)methyl)-2-methylphenyl)-N′,N′dimethylaminosulfamide; Compound 2.078, which is (R)—N-(3-((3-(isoquinolin-5-ylamino)pyrrolidin-1-yl)methyl)-2-methylphenyl)methanesulfonamide; Compound 2.079, which is (R)—N-(3-((3-(isoquinolin-5-ylamino)pyrrolidin-1-yl)methyl)-2-methylphenyl)-N′,N′ dimethylaminosulfamide; Compound 2.082, which is (R)—N-(1-((2-(methylthio)pyrimidin-4-yl)methyl)pyrrolidin-3-yl)isoquinolin-5-amine; Compound 2.096, which is (R)—N-(3-((3-(isoquinolin-5-ylamino)pyrrolidin-1-yl)methyl)-2-methoxyphenyl)methanesulfonamide; Compound 2.097, which is (R)—N-(3-((3-(isoquinolin-5-ylamino)pyrrolidin-1-yl)methyl)-2-methoxyphenyl)-N′,N′ dimethylaminosulfamide; or Compound 2.099, which is (R)-2-(5-((3-(isoquinolin-5-ylamino)pyrrolidin-1-yl)methyl)-2-methylphenoxy)acetamide.

6. A method of treating rheumatoid arthritis or inflammatory bowel disease, comprising the steps of first identifying a subject suffering from rheumatoid arthritis or inflammatory bowel disease, then administering to the subject an effective amount of a compound of Formula II to treat rheumatoid arthritis or inflammatory bowel disease; 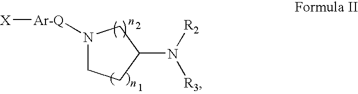
wherein:
Q is C═O, SO₂, or (CR₄R₅)ₙ₃;
n₁ is 1, 2, or 3;
n₂ is 1 or 2;
n3 is 0, 1, 2, or 3;
wherein the ring represented by 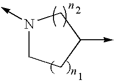
is optionally substituted by alkyl, halo, oxo, OR₆, NR₆R₇, or SR₆;
R₂ is R₂-1 or R₂-2, optionally substituted: 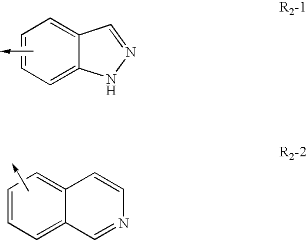
Ar is a monocyclic or bicyclic aryl or heteroaryl ring;
X is from 1 to 3 substituents on Ar, each independently in the form Y-Z, in which Z is attached to Ar;
Y is one or more substituents on Z, and each is independently selected from the group consisting of H, halogen, OR₈, NR₈R₉, NO₂, SR₈, SOR₈, SO₂R₈, SO₂NR₈R₉, NR₈SO₂R₉, OCF₃, CONR₈R₉, NR₈C(═O)R₉, NR₈C(═O)OR₉, OC(═O)NR₈R₉, and NR₈C(═O)NR₉R₁₀;
Z is alkenyl, alkynyl, aryl, arylalkyl, arylalkenyl, arylalkynyl, heteroaryl, heteroarylalkyl, heteroarylalkenyl, heteroarylalkynyl, cycloalkyl, cycloalkenyl, cycloalkylalkyl, cycloalkylalkenyl, cycloalkylalkynyl, heterocycle, (heterocycle)alkyl, (heterocycle)alkenyl, and (heterocycle)alkynyl;
R₃-R₇ are independently H, alkyl, alkenyl, alkynyl, cycloalkyl, cycloalkenyl, cycloalkylalkyl, cycloalkylalkenyl, or cycloalkylalkynyl, optionally substituted;
R₈ is H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle; optionally substituted by one or more halogen or heteroatom-containing substituents selected from the group consisting of OR₁₁, NR₁₁R₁₂, NO₂, SR₁₁, SOR₁₁, SO₂R₁₁, SO₂NR₁₁R₁₂, NR₁₁SO₂R₁₂, OCF₃, CONR₁₁R₁₂, NR₁₁C(═O)R₁₂, NR₁₁C(═O)OR₁₂, OC(═O)NR₁₁R₁₂, and NR₁₁C(═O)NR₁₂R₁₃;
R₉ and R₁₀ are independently H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle; optionally substituted by one or more halogen or heteroatom-containing substituents selected from the group consisting of OR₁₄, NR₁₄R₁₅, NO₂, SR₁₄, SOR₁₄, SO₂R₁₄, SO₂NR₁₄R₁₅, NR₁₄SO₂R₁₅, OCF₃, CONR₁₄R₁₅, NR₁₄C(═O)R₁₅, NR₁₄C(═O)OR₁₅, OC(═O)NR₁₄R₁₅, and NR₁₄C(═O)NR₁₅R₁₆;
wherein any two of the groups R₈, R₉ and R₁₀ are optionally joined with a link selected from the group consisting of bond, —O—, —S—, —SO—, —SO₂—, and —NR₁₇— to form a ring; and
R₁₁-R₁₇ are independently H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle.

7. The method according to claim 6, wherein said compound of Formula II is a compound of Formula IIa, IIb, or IIc: 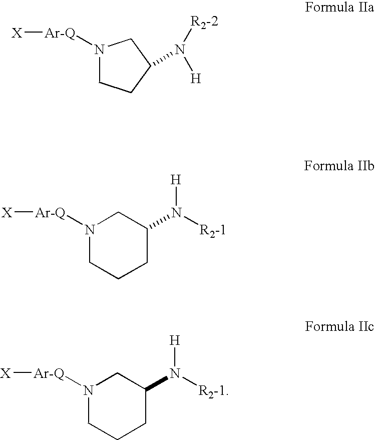
wherein Ar is phenyl, a 6,5-fused bicyclic heteroaryl ring, or a 6,6-fused bicyclic heteroaryl ring; Ar is substituted by 1 or 2 substituents X, and Q is CH₂.

8. The method according to claim 6, wherein said compound is Compound 1.076, which is (R)—N-(1-(4-ethynylbenzyl)piperidin-3-yl)-1H-indazol-5-amine; Compound 1.077, which is (S)—N-(1-(4-ethynylbenzyl)piperidin-3-yl)-1H-indazol-5-amine; Compound 1.153, which is (S)—N-(1-(3-ethynylbenzyl)piperidin-3-yl)-1H-indazol-5-amine; Compound 1.186, which is (S)—N-(1-(3-cyclopropylbenzyl)piperidin-3-yl)-1H-indazol-5-amine; Compound 1.193, which is (R)—N-(1-(3-ethynylbenzyl)piperidin-3-yl)-1H-indazol-5-amine; Compound 1.206, which is (R)—N-(1-(4-cyclopropylbenzyl)piperidin-3-yl)-1H-indazol-5-amine; or Compound 2.031, which is (R)—N-(1-(4-ethynylbenzyl)pyrrolidin-3-yl)isoquinolin-5-amine.

9. A method of treating rheumatoid arthritis or inflammatory bowel disease, comprising the steps of first identifying a subject suffering from rheumatoid arthritis or inflammatory bowel disease, then administering to the subject an effective amount of a compound of Formula II to treat rheumatoid arthritis or inflammatory bowel disease; 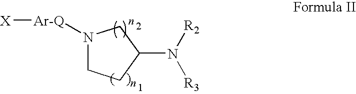
wherein:
Q is C═O, SO₂, or (CR₄R₅)ₙ₃;
n₁ is 1, 2, or 3;
n₂ is 1 or 2;
n₃ is 0, 1, 2, or 3;
wherein the ring represented by 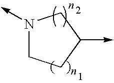
is optionally substituted by alkyl, halo, oxo, OR₆, NR₆R₇, or SR₆;
R₂ is R₂-1 or R₂-2, optionally substituted: 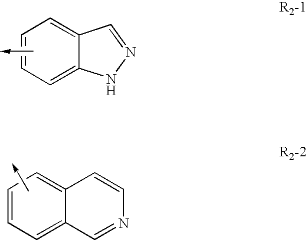
Ar is a monocyclic or bicyclic aryl or heteroaryl ring;
X is from 1 to 3 substituents on Ar, each independently in the form Y-Z, in which Z is attached to Ar;
Y is one or more substituents on Z, and each is independently OR₈, NR₈R₉, NO₂, SR₈, SOR₈, SO₂R₈, SO₂NR₈R₉, NR₈SO₂R₉, OCF₃, CONR₈R₉, NR₈C(═O)R₉, NR₈C(═O)OR₉, OC(═O)NR₈R₉, or NR₈C(═O)NR₉R₁₀,
Z is alkyl, alkenyl, alkynyl, aryl, arylalkyl, arylalkenyl, arylalkynyl, heteroaryl, heteroarylalkyl, heteroarylalkenyl, heteroarylalkynyl, cycloalkyl, cycloalkenyl, cycloalkylalkyl, cycloalkylalkenyl, cycloalkylalkynyl, heterocycle, (heterocycle)alkyl, (heterocycle)alkenyl, or (hetero cycle) alkynyl;
R₃-R₇ are independently H, alkyl, alkenyl, alkynyl, cycloalkyl, cycloalkenyl, cycloalkylalkyl, cycloalkylalkenyl, or cycloalkylalkynyl, optionally substituted;
R₈ is H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle; optionally substituted by one or more halogen or heteroatom-containing substituents selected from the group consisting of OR₁₁, NR₁, R₁₂, NO₂, SR₁₁, SOR₁₁, SO₂R₁₁, SO₂NR₁₁R₁₂, NR₁₁SO₂R₁₂, OCF₃, CONR₁₁R₁₂, NR₁₁C(═O)R₁₂, NR₁₁C(═O)OR₁₂, OC(═O)NR₁₁R₁₂, and NR₁₁C(═O)NR₁₂R₁₃;
R₉ and R₁₀ are independently H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle; optionally substituted by one or more halogen or heteroatom-containing substituents selected from the group consisting of OR₁₄, NR₁₄R₁₅, NO₂, SR₁₄, SOR₁₄, SO₂R₁₄, SO₂NR₁₄R₁₅, NR₁₄SO₂R₁₅, OCF₃, CONR₁₄R₁₅, NR₁₄C(═O)R₁₅, NR₁₄C(═O)OR₁₅, OC(═O)NR₁₄R₁₅, or NR₁₄C(═O)NR₁₅R₁₆;
wherein any two of the groups R₈, R₉ and R₁₀ are optionally joined with a link selected from the group consisting of bond, —O—, —S—, —SO—, —SO₂—, and —NR₁₇— to form a ring; and
R₁₁-R₁₇ are independently H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle;
with the proviso that when Z is selected from the group consisting of alkyl, alkenyl, and alkynyl, and Y falls on the carbon by which Z is attached to Ar, then Y contains at least one nitrogen or sulfur atom.

10. The method according to claim 9, wherein Ar is a heteroaryl.

11. The method according to claim 9, wherein said compound of Formula II is a compound of Formula IIa, IIb, or IIc: 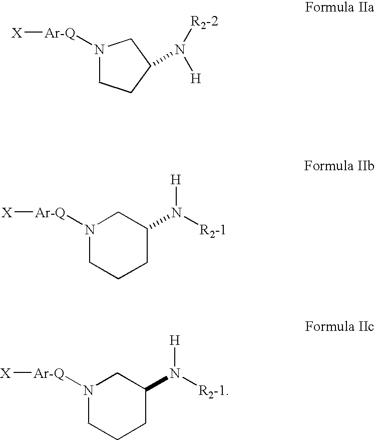
wherein Ar is phenyl, a 6,5-fused bicyclic heteroaryl ring, or a 6,6-fused bicyclic heteroaryl ring; Ar is substituted by 1 or 2 substituents X, and Q is CH₂.

12. The method according to claim 9, wherein said compound is Compound 1.108, which is (R)-2-(6-((3-(1H-indazol-5-ylamino)piperidin-1-yl)methyl)-1H-indol-1-yl)ethanol; Compound 1.109, which is (S)-2-(6-((3-(1H-indazol-5-ylamino)piperidin-1-yl)methyl)-1H-indol-1-yl)ethanol; Compound 1.162, which is (R)-2-(5-((3-(1H-indazol-5-ylamino)piperidin-1-yl)methyl)-1H-indol-1-yl)acetamide; Compound 1.165, which is (S)-2-(5-((3-(1H-indazol-5-ylamino)piperidin-1-yl)methyl)-H-indol-1-yl)acetamide; Compound 1.176, which is (S)-tert-butyl 3-((4-(1H-indazol-5-ylamino)piperidin-1-yl)methyl)benzylcarbamate; Compound 1.197, which is (S)—N-(1H-indazol-5-ylamino)piperidin-1-yl)methyl)benzyl)acetamide; Compound 1.217, which is (S)-2-(6-((3-(1H-indazol-5-ylamino)piperidin-1-yl)methyl)indolin-1-yl)ethanol; Compound 1.223, which is (S)-(4-((3-(1H-indazol-5-ylamino)piperidin-1-yl)methyl)phenyl)methanol; Compound 1.273, which is (R)-2-(3-((3-(1H-indazol-5-ylamino)piperidin-1-yl)methyl)-1H-indol-1-yl)ethanol; Compound 2.058, which is (R)-2-(6-((3-(isoquinolin-5-ylamino)pyrrolidin-1-yl)methyl)-1H-indol-1-yl)acetamide; Compound 2.059, which is (R)-2-(5-((3-(isoquinolin-5-ylamino)pyrrolidin-1-yl)methyl)-1H-indol-1-yl)acetamide; Compound 2.060, which is (R)-2-(6-((3-(isoquinolin-5-ylamino)pyrrolidin-1-yl)methyl)-1H-indol-1-yl)ethanol; Compound 2.066, which is (R)-2-(5-((3-(isoquinolin-5-ylamino)pyrrolidin-1-yl)methyl)-1H-indol-1-yl)ethanol; or Compound 2.072, which is (R)-2-(3-((3-(isoquinolin-5-ylamino)pyrrolidin-1-yl)methyl)-1H-indol-1-yl)ethanol.

13. A method of treating rheumatoid arthritis or inflammatory bowel disease, comprising the steps of first identifying a subject suffering from rheumatoid arthritis or inflammatory bowel disease, then administering to the subject an effective amount of a compound of Formula II to treat rheumatoid arthritis or inflammatory bowel disease; 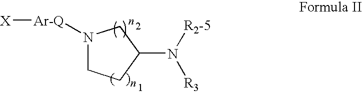
wherein:
Q is C═O, SO₂, or (CR₄R₅)ₙ₃;
n₁ is 1, 2, or 3;
n₂ is 1 or 2;
n₃ is 0, 1, 2, or 3;
wherein the ring represented by 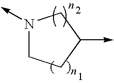
is optionally substituted by alkyl, halo, oxo, OR₆, NR₆R₇, or SR₆;
R₂-5 is 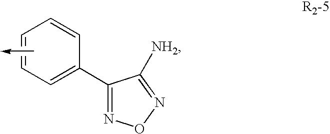
optionally substituted;
Ar is a monocyclic or bicyclic aryl or heteroaryl ring;
X is from 1 to 3 substituents on Ar, each independently in the form Y-Z, in which Z is attached to Ar;
Y is one or more substituents on Z, and each is independently selected from the group consisting of H, halogen, OR₈, NR₈R₉, NO₂, SR₈, SOR₈, SO₂R₈, SO₂NR₈R₉, NR₈SO₂R₉, OCF₃, CONR₈R₉, NR₈C(═O)R₉, NR₈C(═O)OR₉, OC(═O)NR₈R₉, and NR₈C(═O)NR₉R₁₀;
Z is independently selected from the group consisting of absent, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, heterocycle, (heterocycle)alkyl, (heterocycle)alkenyl, and (heterocycle)alkynyl;
R₃-R₇ are independently H, alkyl, alkenyl, alkynyl, cycloalkyl, cycloalkenyl, cycloalkylalkyl, cycloalkylalkenyl, or cycloalkylalkynyl, optionally substituted;
R₈ is H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alynyl, or heterocycle; optionally substituted by one or more halogen or heteroatom-containing substituents selected from the group consisting of OR₁₁, NR₁₁R₁₂, NO₂, SR₁₁, SOR₁₁, SO₂R₁₁, SO₂NR₁₁R₁₂, NR₁₁SO₂R₁₂, OCF₃, CONR₁₁R₁₂, NR₁₁C(═O)R₁₂, NR₁₁C(═O)OR₁₂, OC(═O)NR₁, R₁₂, and NR₁₁C(═O)NR₁₂R₁₃;
R₉ and R₁₀ are independently H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle; optionally substituted by one or more halogen or heteroatom-containing substituents selected from the group consisting of OR₁₄, NR₁₄R₁₅, NO₂, SR₁₄, SOR₁₄, SO₂R₁₄, SO₂NR₁₄R₁₅, NR₁₄SO₂R₁₅, OCF₃, CONR₁₄R₁₅, NR₁₄C(═O)R₁₅, NR₁₄C(═O)OR₁₅, OC(═O)NR₁₄R₁₅, and NR₁₄C(═O)NR₁₅R₁₆;
wherein any two of the groups R₈, R₅ and R₁₀ are optionally joined with a link selected from the group consisting of bond, —O—, —S—, —SO—, —SO₂—, and —NR₁₇— to form a ring; and
R₁₁-R₁₇ are independently H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, or heterocycle.

14. A method of treating rheumatoid arthritis or inflammatory bowel disease, comprising the steps of first identifying a subject suffering from rheumatoid arthritis or inflammatory bowel disease, then administering to the subject an effective amount of a compound of Formula Ia, Ib, or Ic to treat rheumatoid arthritis or inflammatory bowel disease; 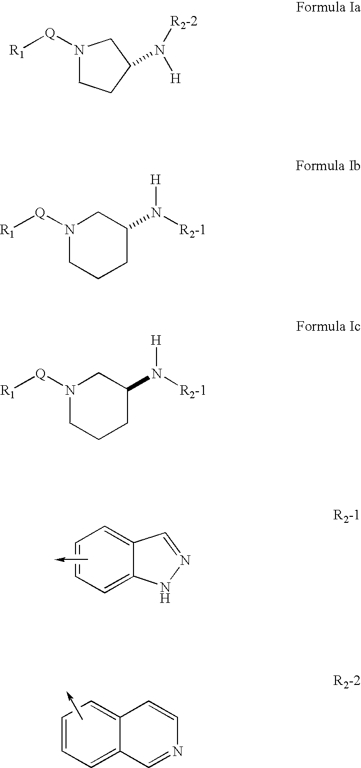
wherein R₁ is phenyl, thiophene, 6,5-fused bicyclic heteroaryl ring, or 6,6-fused bicyclic heteroaryl ring, R₁ is either unsubstituted or is optionally substituted with 1, 2 or 3 substituents independently selected from halogen, methyl, ethyl, hydroxyl, methoxy, or ethoxy;
Q is C═O, SO₂, or (CR₄R₅)ₙ₃;
R₂-1 and R₂-2 are optionally substituted;
R₄ and R₅ are independently H, alkyl, cycloalkyl, optionally substituted.

15. The method according to claim 14, wherein R₁ is 3-substituted phenyl, 4-substituted phenyl, 3,4-disubstituted phenyl, or 6,5-fused bicyclic heteroaryl ring.

16. The method according to claim 15, wherein R₁ is benzofuran, benzothiophene, indole, and benzimidazole.

17. The method according to claim 14, wherein R₄ and R₅ are independently H or an unsubstituted alkyl.

18. The method according to claim 14, wherein said compound of Formula Ia is Compound 2.025, which is (R)—N-(1-(4-methylbenzyl)pyrrolidin-3-yl)isoquinolin-5-amine; Compound 2.046, which is (R)—N-(1-benzylpyrrolidin-3-yl)isoquinolin-5-amine; Compound 2.047, which is (R)—N-(1-(4-methoxybenzyl)pyrrolidin-3-yl)isoquinolin-5-amine; Compound 2.055, which is (R)—N-(1-(benzofuran-5-ylmethyl)pyrrolidin-3-yl)isoquinolin-5-amine; Compound 2.057, which is (R)—N-(1-((1H-indol-6-yl)methyl)pyrrolidin-3-yl)isoquinolin-5-amine; Compound 2.061, which is (R)-3-((3-(isoquinolin-5-ylamino)pyrrolidin-1-yl)methyl)phenol; or Compound 2.065, which is (R)—N-(1-((1H-indol-5-yl)methyl)pyrrolidin-3-yl)isoquinolin-5-amine.

19. The method according to claim 14, wherein said compound of Formula Ic is Compound 1.079, which is (S)—N-(1-(4-methoxybenzyl)piperidin-3-yl)-1H-indazol-5-amine; Compound 1.141, which is (S)—N-(1-(4-chlorobenzyl)piperidin-3-yl)-1H-indazol-5-amine; Compound 1.148, which is (S)—N-(1-((1H-indol-6-yl)methyl)piperidin-3-yl)-1H-indazol-5-amine; Compound 1.149, which is (S)—N-(1-((1H-indol-5-yl)methyl)piperidin-3-yl)-1H-indazol-5-amine; Compound 1.150, which is (S)—N-(1-(benzofuran-5-ylmethyl)piperidin-3-yl)-1H-indazol-5-amine; Compound 1.155, which is (S)—N-(1-(2,4-dimethylbenzyl)piperidin-3-yl)-1H-indazol-5-amine; Compound 1.156, which is (S)—N-(1-(2,3-dimethylbenzyl)piperidin-3-yl)-1H-indazol-5-amine; Compound 1.163, which is (S)-3-((3-(1H-indazol-5-ylamino)piperidin-1-yl)methyl)phenol; Compound 1.164, which is (S)—N-(1-(4-fluorobenzyl)piperidin-3-yl)-1H-indazol-5-amine; Compound 1.166, which is (S)—N-(1-((2,3-dihydrobenzo[b][14]dioxin-6-yl)methyl)piperidin-3-yl)-1H-indazol-5-amine; Compound 1.171, which is (S)—N-(1-(3-methylbenzyl)piperidin-3-yl)-1H-indazol-5-amine; Compound 1.175, which is (S)—N-(1-(benzo[b]thiophen-5-ylmethyl)piperidin-3-yl)-1H-indazol-5-amine; or Compound 1.277, which is (S)—N-(1-(thiophen-3-ylmethyl)piperidin-3-yl)-1H-indazol-5-amine.

20. The method according to claim 14, wherein said compound of Formula Ib is Compound 1.131, which is (R)—N-(1-(benzofuran-5-ylmethyl)piperidin-3-yl)-1H-indazol-5-amine; Compound 1.132, which is (R)—N-(1-(4-chlorobenzyl)piperidin-3-yl)-1H-indazol-5-amine; Compound 1.133, which is (R)—N-(1-(4-methylbenzyl)piperidin-3-yl)-1H-indazol-5-amine; Compound 1.134, which is (R)—N-(1-(4-bromobenzyl)piperidin-3-yl)-1H-indazol-5-amine; Compound 1.135, which is (R)—N-(1-(4-ethylbenzyl)piperidin-3-yl)-1H-indazol-5-amine; Compound 1.136, which is (R)—N-(1-(2,4-dimethylbenzyl)piperidin-3-yl)-1H-indazol-5-amine; Compound 1.137, which is (R)—N-(1-(benzo[b]thiophen-5-ylmethyl)piperidin-3-yl)-1H-indazol-5-amine; Compound 1.138, which is (R)—N-(1-((1H-indol-6-yl)methyl)piperidin-3-yl)-1H-indazol-5-amine; Compound 1.173, which is (R)-5-((3-(1H-indazol-5-ylamino)piperidin-1-yl)methyl)-2-methylphenol; or Compound 1.252, which is (R)—N-(1-((1H-indol-3-yl)methyl)piperidin-3-yl)-1H-indazol-5-amine.
